(19) United States
(12) Patent Application Publication
(10) Pub. No.: US 2023/0398101 A1
(43) Pub. Date: Dec. 14, 2023

(54) Co-agents as Therapy Against Anaerobic Pathogens

(71) Applicant: Fleurir ABX LLC, Raleigh, NC (US)

(72) Inventors: John Lee Pace, Elizabeth City, NC (US); Theresa Lynn Hartsell, Elizabeth City, NC (US)

(73) Assignee: Fleurir ABX LLC, Raleigh, NC (US)

(21) Appl. No.: 17/836,959

(22) Filed: Jun. 9, 2022

(51) Int. Cl. A61K 31/431; A61P 31/04; A61K 45/06; A61K 31/546; A61K 38/17

(52) U.S. Cl. CPC A61K 31/431; A61P 31/04; A61K 45/06; A61K 31/546; A61K 38/1729

(57) ABSTRACT

Co-agent combinations and/or formulations herein unexpectedly display significantly better antimicrobial activity (e.g., more efficacy and/or more potency) against anaerobic pathogens not previously considered targets. With three or more co-agents, selected from a group of a fosfomycin, a diaminopyridine, a sulfonamide, a beta lactam antibacterial, a bacterial beta-lactamase inhibitor, a bacterial fosfomycin-modifying enzyme, and a bacterial peptidoglycan synthesis inhibitor, the therapeutic potential of the three or more co-agents is expanded by targeting a broader spectrum of pathogens. Co-agents, by unexpected synergistic action in an anaerobic environment, are now active and efficacious against difficult to treat pathogenic anaerobes (including anaerobes that cannot utilize oxygen and/or reside in an anaerobic environment, some being inhibited by oxygen), as well as pathogens considered resistant or intolerant to at least one of the co-agents when used singly in an anaerobic environment. Some co-agent combinations and/or formulations contain one or more existing antibiotic agents being repurposed for utility against difficult to treat anaerobic pathogens.

### FIELD OF THE INVENTION

The subject application describes a plurality of novel combinations and/or formulations, each comprising a plurality of agents (co-agents), and each unexpectedly providing an antimicrobial effect against difficult to treat pathogenic anaerobes, including those that cannot utilize oxygen, some being inhibited by oxygen. Each co-agent in a combination or formulation interferes with or inhibits an enzyme of an anaerobic pathogen. The plurality of co-agents are selected for their synergism, displaying and having synergistic action in a manner that facilitates or otherwise promotes sufficient and/or effective antimicrobial activity against difficult to treat anaerobic pathogens in an anaerobic environment, yet many anaerobes are intrinsically resistant to a co-agent when used singly, and many co-agents are inactive against the same anaerobe when the co-agent is used singly.

### BACKGROUND

With a continued rise in more complicated infections as well as ever increasing rates of antibiotic resistance, particularly with highly utilized classes of antibiotics, there remains a need to find effective antimicrobials, effective against pathogenic anaerobic bacteria, and for antimicrobials having a broad spectrum of activity, in order to adequately and effectively combat hard to treat organisms or mixed infections (comprising one or more species of anaerobic pathogens and one or more species of aerobic pathogens). WHO has noted a lack of new antibiotics stemming from a reduction in research and development coupled with a global rise in both antibiotic-resistant pathogens and infections stemming from such resistant pathogens. (World Health Organization, “Lack of new antibiotics threatens global efforts to contain drug-resistant infections,” Jan. 17, 2020, at https://www.who.int/news/item/17-01-2020-lack-of-new-antibiotics-threatens-global-efforts-to-contain-drug-resistant-infections). This has led to an over reliance on certain classes of antibiotics accelerating accumulation of resistance selection and expansion of resistance, which, together are resulting in ever-increasing limitations in treatment options.

Infections from anaerobic microorganisms are on the rise (due, in part, to increases in overall age of populations, increases in the incidence of certain predisposing diseases, such as inflammatory diseases and cancers, which increases risk of intestinal perforations and peritonitis, as well as a rise in number of surgical procedures, accidents, injuries and deeply penetrating wounds, among other reasons). Yet, many current antibiotics used to treat aerobic microorganisms (those able to utilize oxygen for metabolism) are not acceptable for use against anaerobes (e.g., by having little or no effectiveness with regard to inhibition of anaerobic bacterial growth and/or anaerobic bacterial elimination, particularly when used against strict or obligate anaerobes). As such, there are limited treatment options available for utilization against more severe infections, including hospital infections, and there is no predictability regarding effective treatments against pathogenic anaerobes responsible for such infections, including anaerobes identified as strict or obligate anaerobes. Obligate anaerobes when pathogenic are most difficult to treat due to their inability to aerobically metabolize. Obligate anaerobes cannot utilize oxygen, and some are actually inhibited by oxygen, and survive and are pathogenic in the absence of oxygen. Obligate anaerobes depend, instead, on other substances (other than oxygen; i.e., terminal electron acceptors) as well as alternative (different) enzymatic pathways for energy conversion (for growth, metabolism, and activity) as compared with aerobic microorganisms that utilize and convert oxygen for energy production.

Indeed, the pharmacotherapy or treatment plans directed against pathogenic anaerobes (including and/or particularly obligate anaerobes) and/or infections arising or suspected of arising from one or more pathogenic anaerobes remain empiric. Antibiotic options currently utilized against pathogenic anaerobes, and particularly obligate anaerobes, are severely limited. This is due, in part, to ineffectiveness of many antibiotic drugs under anaerobic conditions. With an ever-increasing resistance of microorganisms (including anaerobes) to the few antibiotic agents that are being utilized against anaerobes, even previously used antibiotics are no longer recommended in empiric treatment against such pathogenic anaerobes (including clindamycin, many penicillins, second-generation cephalosporins, quinolones, as examples). An inability to direct therapy against pathogenic anaerobes is a common reason for clinical failure.

There remains a need for direct and novel pharmacotherapy option(s) effective against pathogenic anaerobes, including pathogenic obligate anaerobes. There also remains a need for direct and novel pharmacotherapy option(s) effective against pathogenic anaerobes, including pathogenic obligate anaerobes, in an anaerobic environment and/or a very poorly oxygenated environment (e.g., under low or very low oxygen tension that is not toxic to such pathogens, and/or does not generally promote sustained energy production utilizing oxygen, and/or one provides sources other than oxygen to obtain energy, such as through fermentation). In addition, or as an alternative, there remains a need for pharmacotherapy option(s) effective against not only one or more pathogenic anaerobes, including pathogenic obligate anaerobes, but may be utilized in a mixed population and/or infection thereof, so as to be effective against at least one pathogenic anaerobe as well as one or more pathogenic aerobic microorganisms (Gram-positive aerobe and/or Gram-negative aerobe). In addition, or as an alternative, there remains a need for pharmacotherapy option(s) that provide sustained growth inhibitory activity and/or sustained elimination against one or more pathogenic anaerobes, including one or more pathogenic obligate anaerobes and/or one or more considered resistant to one or more single antibiotic agent when used singly. In addition, or as an alternative, there remains a need for pharmacotherapy option(s) that provide appropriate care against an infection derived from or suspected of arising or developing from one or more pathogenic anaerobes, including one or more pathogenic obligate anaerobes and/or one or more considered resistant to one or more single antibiotic agent when used singly. There also remains a need for pharmacotherapy option(s) that penetrate a blood-brain barrier (e.g., in a subject in need thereof) and, upon and/or after penetrating the blood-brain barrier (e.g., in a subject in need thereof), have activity against one or more pathogenic anaerobes, including one or more pathogenic obligate anaerobes and/or one or more considered resistant to one or more single antibiotic agent when used singly.

One or more and/or all needs are met by one or more combinations and/or formulations herein below, which, when utilized, provide one or more benefits described herein, due to a synergistic action of the agents utilized in said one or more combinations and/or formulations.

### SUMMARY

Herein are a plurality of unpredictable combinations of antimicrobial agents (co-agents) for utilization against one or more type or species of pathogenic anaerobic microorganisms (anaerobes), in which each unpredictable combination would not have been predicted to be effective against said one or more type or species of pathogenic anaerobes, because, in part, at least one co-agent in the unpredictable combination, on its own, is either ineffective as an antimicrobial against said one or more type or species of pathogenic anaerobes in an anaerobic environment and/or is not active against the one or more type or species of pathogenic anaerobes (due to an acquired resistance or intrinsic resistance of the one or more type or species of pathogenic anaerobes to the at least one co-agent). In addition, or alternatively, at least one unpredictable combination would not be predicted to be effective against said one or more type or species of pathogenic anaerobe, because, in part, said pathogenic anaerobes are in an anaerobic environment. In addition, or an alternative, at least one unpredictable combination would not have been predicted to be effective against said one or more type or species of pathogenic anaerobes, because, in part, one or more type or species of pathogenic anaerobes are intrinsically resistant to at least one co-agent in at least one unpredictable combination. With the unpredictable combination of co-agents, said combination is not only effective against said one or more type or species of pathogenic anaerobes in an anaerobic environment, the action of the combination is unexpectedly synergistic, having or causing an antimicrobial effect (when as the combination) and at least sufficiently inhibiting growth of said one or more pathogenic anaerobes.

In one or more embodiments, pathogenic anaerobes are at least one type or species of pathogenic obligate anaerobe. In one or more embodiments, pathogenic anaerobes are at least one type or species of Gram-negative obligate anaerobe (GNOA). In one or more embodiments, pathogenic anaerobes are at least one type or species of Gram-positive obligate anaerobe (GPOA). In one or more embodiments, pathogenic anaerobes are at least one type or species of drug-resistant pathogenic anaerobe. In one or more embodiments, pathogenic anaerobes are at least one type or species of drug-resistant pathogenic anaerobe, in which the drug (that the anaerobe is resistant to) is at least one co-agent in the unpredictable combination herein, when said co-agent is used alone against that pathogenic anaerobe. In one or more embodiments, pathogenic anaerobes are at least one type or species of intrinsically resistant drug-resistant pathogenic anaerobe, in which the at least one type or species of pathogenic anaerobe is intrinsically resistant to at least one co-agent in the unpredictable combination herein. For intrinsic resistance, a drug (or co-agent) is one that is not expected to possess antimicrobial activity against the at least one type or species of pathogenic anaerobe in its entirety. Intrinsic resistance (or intrinsically resistant) may also be referred to as expected phenotypic resistance or intrinsic non-susceptibility, and is not acquired resistance. In one or more embodiments, pathogenic anaerobes are at least one type or species of drug-resistant GNOA, in which drug resistance is intrinsic or acquired. In one or more embodiments, pathogenic anaerobes are at least one type or species of drug-resistant GPOA, in which drug resistance is intrinsic or acquired. In one or more embodiments, pathogenic anaerobes are at least two types or species of microorganisms comprising at least one type or species of pathogenic anaerobe and at least one type or species of pathogenic aerobe, also referred to as a mixed population of organisms. In one or more embodiments, pathogenic anaerobes are a mixed population containing at least one type or species of drug-resistant pathogenic anaerobe, in which the drug (that the anaerobe is resistant to) is at least one co-agent in the unpredictable combination. In one or more embodiments, pathogenic anaerobes are at least one type or species of pathogenic anaerobe intrinsically resistant to at least two co-agents in the unpredictable combination. In one or more embodiments, pathogenic anaerobes are at least one type or species of pathogenic anaerobe intrinsically resistant to at least three co-agents in the unpredictable combination. Being intrinsically resistant to at least one, or at least two, or at least three co-agents in the unpredictable combination means that such agent would never be (or have been) considered effective in any of the one or more unpredictable combinations and/or formulations described herein. It is understood that any combination of pathogenic anaerobes as described herein (or above) may be targeted by an unpredictable combination and/or formulation herein.

As used herein, susceptibility and/or resistance to at least one or at least two or at least three or at least four co-agents in an unpredictable combination and/or formulation herein may be identified by susceptibility breakpoints, set and/or identified by a practiced organization that establishes standards, such as EUCAST (European Committee on Antimicrobial Susceptibility Testing, Vaxjo, Sweden) or CLSI (Clinical and Laboratory Standards Institute, Pennsylvania, U.S.A). Acceptable standardized testings for anaerobes may be a culture-based or agar-based in vitro antimicrobial susceptibility test (AST) by CLSI; EUCAST does not presently provide a standardized test for anaerobes.

As used herein, pathogenic anaerobes are any type or species of anaerobes described above or below, or any combination thereof, requiring a pharmacologic interference or intervention with at least one unpredictable antimicrobial combination and/or formulation herein, in which such a pharmacologic interference or intervention is to inhibit or reduce or abate or control or eliminate pathogenicity (which is due at least in part to one or more pathogenic anaerobes) and/or to inhibit or reduce or abate or control or eliminate an infection caused by or suspected of being caused by one or more pathogenic anaerobes herein (e.g., in a host or subject having or suspected of having an infection). When referencing any of a term such as providing and/or treating and/or delivering and/or conveying and/or presenting and/or dispensing in a manner involving a pharmacologic interference or intervention with any combination and/or formulation herein (comprising a plurality of co-agents or active co-agents or synergists), the pharmacologic interference or intervention will include unpredictable combination and/or formulation herein, directed towards or directed against at least one or more type and/or species of pathogenic anaerobes as described herein, whether or not the term “pathogenic” (or any suitable equivalent term of art as understood by those of ordinary skill in the art) is included in the wording or in a claim, and/or whether or not the phrase “one or more type or species” or “one or more type and/or species” (provided interchangeably herein in reference to pathogenic anaerobes) is included in the wording or in a claim, and/or whether or not the phrase “pharmacologic interference or intervention” (or any suitable equivalent term of art as understood by those of ordinary skill in the art) is included in the wording or in a claim.

Unpredictable combinations and/or formulations (each comprising a plurality of co-agents) will each unexpectedly exhibit or display a response and antimicrobial activity as described herein. Antimicrobial activity is sufficient and/or suitable against one or more pathogenic anaerobes herein, which may include pathogenic anaerobes that cannot utilize oxygen and/or are inhibited by oxygen; and such activity is, in many embodiments, at least as therapeutically beneficial (efficacious) as an alternative and known antibiotic presently approved for use against an aerobic bacteria. In some embodiments, activity is or includes a sufficient and more potent activity than an alternative and known antibiotic presently approved for use against an aerobic bacteria, and which is inactive against pathogenic anaerobes that cannot utilize oxygen, or are inhibited by oxygen. Activity of the unpredictable combinations and/or formulations herein are due to co-agent synergism, which, as further disclosed, provides unexpected and unpredicted antimicrobial activity against one or more pathogenic anaerobes, including in an anaerobic environment. Synergistic action positively influences and imparts one or more responses and activities of co-agents against one or more (type and/or species of) pathogenic anaerobes. Activity that is manifested or caused by an unpredictable combination and/or formulation herein, which is active against an anaerobic pathogen or an infection caused or suspected of being caused by the anaerobic pathogen, the activity being one or any combination of: (i) growth inhibitory activity against one or more pathogenic anaerobes; (ii) sustained growth inhibitory activity against one or more pathogenic anaerobes; (iii) elimination (and/or destruction) of one or more pathogenic anaerobes; (iv) sustained elimination (and/or destruction) of one or more pathogenic anaerobes; (v) preventative activity against formation of microbial colonies of one or more pathogenic anaerobes; (vi) sustained preventative activity against formation of microbial colonies of one or more pathogenic anaerobes; (vii) alleviation of infection caused by, derived from or suspected of being caused by one or more pathogenic anaerobes (e.g., in a subject in need); (viii) control of infection caused by, derived from or suspected of being caused by one or more pathogenic anaerobes (e.g., in a subject in need); (ix) prevention of infection that would be caused by or derived from or suspected of developing from one or more pathogenic anaerobes (e.g., in a subject in need thereof; e.g., pre- and/or post-surgery); (x) reduction in infection rate of an infection caused by or derived from or suspected of being caused by one or more pathogenic anaerobes (e.g., in a subject in need). Accordingly, synergistic action and antimicrobial activity of a co-agent combination and/or formulation herein may be sufficient and/or effective as pharmacotherapy, and utilized to treat an infection, prevent a possible infection (e.g., associated with a surgery), reduce systemic hospital infection rate, reduce adjustments/changes to initial antibiotic treatment or therapy, reduce or eliminate empiric antibiotic therapy, improve overall hospital outcome indicators, reduce hospital length of stay, and/or reduce length of antibiotic treatment or therapy.

Unexpectedly, in some embodiments, an unpredictable co-agent combination and/or formulation herein may revive a therapeutic potential of at least one co-agent, such that the co-agent may now be provided for a new use and/or broader spectrum of activity and/or better outcome, when at least one co-agent provided with the unpredictable co-agent combination and/or formulation is a known antibiotic currently approved for use as an antibiotic.

Unexpectedly, unpredictable co-agent combinations and/or formulations herein have synergistic action and effective antimicrobial activity against one or more pathogenic anaerobes (e.g., manifested as any one or more of activities (i) to (x)), being active against an unexpected group of pathogens, which include: obligate anaerobes (Gram-positive and/or Gram-negative); obligate anaerobes in a mixed population; intrinsically resistant drug-resistant obligate anaerobes; non-susceptible anaerobes. Unexpectedly, unpredictable co-agent combinations and/or formulations herein (having synergistic action and antimicrobial activity against one or more anaerobes manifested as one or more of activities (i) to (x)), are active in an anaerobic environment, yet are selectively active, being unexpectedly inactive against certain facultative (nonresponsive) anaerobes, those identified by others of skill in the relevant field as beneficial or mutualistic anaerobes (e.g., commensal to a microbiome or to a normal or non-diseased microflora of a mammalian host). The nonresponsive facultative anaerobes are any of Lactobacillus spp., Leuconostoc spp., and Pediococcus spp., none found responsive to unpredictable co-agent combinations and/or formulations herein, when a co-agent combination and/or formulation comprises at least the following co-agents: (1) a fosfomycin (F) having on its own antibacterial property (against an aerobic bacteria in aerobic environ), including any suitable phosphate, salt, acid, amine, and/or ester thereof, any of which interfere with or inhibit microbial (bacterial) UDP-N-acetylglucosamine (UDP-GlcNAc) enolpyruvyl transferase (or MurA enzyme) or an inhibitor of a microbial (bacterial) MurA enzyme; (2) a diaminopyrimidine (D) having on its own antibacterial property (against an aerobic bacteria in aerobic environ), including any suitable phosphate, salt, acid, and/or ester thereof, any of which interfere with or inhibit microbial (bacterial) dihydrofolate reductase (DHFR) or an inhibitor of microbial (bacterial) DHFR; and (3) a sulfonamide (S) having on its own antibacterial property (against an aerobic bacteria in aerobic environ), including any suitable phosphate, salt, acid, and/or ester thereof, any of which interfere with or inhibit microbial (bacterial) dihydropteroate synthase (DHPS) or an inhibitor of microbial (bacterial) DHPS.

In some forms, unpredictable co-agent combinations and/or formulations herein, that have synergistic action and antimicrobial activity against one or more pathogenic anaerobes (e.g., manifested as any one or more of activities (i) to (x)), have a post-antibiotic effect (PAE) that is not predicted, as is evidenced by in vitro testings against responsive facultative anaerobes (and when compared to PAE of a co-agent tested singly). In one or more embodiments, PAE is longer for a co-agent combination and/or formulation herein against a responsive anaerobe, as compared with PAE of a co-agent when provided alone (singly) against the responsive anaerobe. In one or more embodiments, PAE after delivery of a co-agent combination and/or formulation is sufficiently same or similar to PAE of a co-agent (from the combination and/or formulation), when the co-agent is provided alone (singly) against a responsive facultative anaerobe, and that sufficiently same or similar PAE for the co-agent combination and/or formulation may occur at a lower concentration of at least one co-agent or all co-agents (when said co-agent is provided singly). PAE of a co-agent combination and/or formulation may be thirty minutes (min) longer, or one hour longer, or two hours longer, or more than two hours longer than PAE of a co-agent of the combination when provided singly. PAE of a co-agent combination and/or formulation may be unexpectedly longer than would be predicted based on PAE of one co-agent from the combination (when provided singly) or a co-agent duo from the combination (when provided as a duo) against a same pathogenic anaerobe. A responsive pathogenic anaerobe may be a facultative anaerobe. A responsive pathogenic anaerobe may be an obligate anaerobe.

In some forms, unexpectedly, an unpredictable co-agent combination and/or formulation herein that has synergistic action and sufficient and/or effective antimicrobial activity against one or more pathogenic anaerobes herein (e.g., manifested as one or more of activities (i) to (x)), exhibits better antimicrobial activity (or more potency) than an individual co-agent from the combination and/or formulation when that co-agent acts alone or singly against the same pathogenic anaerobes (which may be assessed with an in vitro anaerobic AST). In some forms, unexpectedly, an unpredictable co-agent combination and/or formulation herein that has synergistic action and sufficient and/or effective antimicrobial activity against one or more pathogenic anaerobes herein (e.g., as any one or more of activities (i) to (x)), exhibits better antimicrobial activity (or more potency) than a co-agent duo from the combination and/or formulation when that duo is provided against the same pathogenic anaerobes (which may be assessed with an in vitro anaerobic AST). A benefit of co-agent combinations and/or formulations herein includes meaningful efficacy (potency) against a pathogenic anaerobe, particularly one that cannot utilize oxygen and/or is inhibited by oxygen, as compared with activity of at least one co-agent from the combination and/or formulation when used singly against the pathogenic anaerobe (which may be assessed with an in vitro anaerobic AST). Another benefit of co-agent combinations and/or formulations herein includes safe and effective activity against a pathogenic anaerobe, particularly one that cannot utilize oxygen and/or is inhibited by oxygen, as compared with a currently approved antibiotic when used as prescribed against the same pathogenic anaerobe in an anaerobic environment (which may be assessed with an in vitro anaerobic AST). Another benefit of co-agent combinations and/or formulations herein includes inducing reversal on co-agent resistance of one or more pathogenic anaerobes, particularly those that cannot utilize oxygen and/or are inhibited by oxygen and are resistant or not susceptible to the co-agent from the combination and/or formulation when that co-agent is used singly against the pathogenic anaerobe (which may be assessed with an in vitro anaerobic AST). In some embodiments, such pathogenic anaerobes will include at least one type or species of obligate anaerobe in an anaerobic environment. In some embodiments, such pathogenic anaerobes will not include a nonresponsive facultative anaerobe of Lactobacillus spp., Leuconostoc spp., and/or Pediococcus spp. that do not respond (are not susceptible) to co-agent combinations and/or formulations herein.

One or more unpredictable co-agent combinations and/or formulations herein, having synergistic action and sufficient and/or effective antimicrobial activity against one or more pathogenic anaerobes (e.g., manifested as any one or more of activities (i) to (x)), when provided to a pathogenic anaerobe herein, restores activity (or potency) of at least one co-agent in the co-agent combination and/or formulation, in which the at least one co-agent is not active against the pathogenic anaerobe when used singly (or as a duo) (which may be assessed with an in vitro anaerobic AST). Some unpredictable co-agent combinations and/or formulations herein, having synergistic action and sufficient and/or effective antimicrobial activity against one or more pathogenic anaerobes (e.g., manifested as any one or more of activities (i) to (x)), when provided to a pathogenic anaerobe herein, establishes activity of at least one co-agent in the co-agent combination and/or formulation, in which the at least one co-agent is not active against the pathogenic anaerobe when used singly (or as a duo) (which may be assessed with an in vitro anaerobic AST). Lack of activity (or inadequate potency) of at least one co-agent against the pathogenic anaerobe when acting individually (or as a duo) may be due to its intrinsic resistance, an acquired resistance, and/or phenotype of the pathogenic anaerobe. In some embodiments, a reversing or restoring or establishing activity of novel co-agent combinations and/or formulations herein is due, at least in part, to co-agent synergism between or amongst co-agents, causing synergistic action of co-agents where synergy could not be or was not previously present. In some embodiments, a reversing or restoring or establishing activity of at least one co-agent in a novel co-agent combination and/or formulation herein causes or provides in an anaerobic environment the sufficient and/or effective antimicrobial activity against one or more pathogenic anaerobe, which may include those that cannot utilize oxygen and/or are inhibited by oxygen, where the sufficient and/or effective antimicrobial activity against the one or more pathogenic anaerobe was not previously observed by the at least one co-agent when used anaerobically and singly (or as a duo).

In some embodiments, an unpredictable co-agent combination and/or formulation has synergistic action and sufficient and/or effective antimicrobial activity against one or more pathogenic anaerobes (e.g., manifested as any one or more of activities (i) to (x)), when co-dosed in a same period or in an overlapping period, and co-dosing provides good antimicrobial efficacy and/or better antimicrobial efficacy as compared with at least one co-agent in the combination when that at least one co-agent is used singly (or as a duo) against the pathogenic anaerobe. In some embodiments, such pathogenic anaerobes will include at least one type or species of obligate anaerobe in an anaerobic environment. In some embodiments, such pathogenic anaerobes will not include a nonresponsive facultative anaerobe of Lactobacillus spp., Leuconostoc spp., and/or Pediococcus spp. that do not respond (are not susceptible) to co-agent combinations and/or formulations herein.

In some embodiments, a co-agent combination and/or formulation herein comprises a plurality of active co-agents, the plurality consisting of, consisting essentially of, or comprising three active co-agents. In some embodiments, the plurality of active co-agents consists of, consists essentially of, or comprises four active co-agents. In some embodiments, the plurality of active co-agents consists of, consists essentially of, or comprises five active co-agents. In some embodiments, the plurality of active co-agents consists of, consists essentially of, or comprises six active co-agents. In some embodiments, the plurality of active co-agents consists of, consists essentially of, or comprises from three active co-agents and up to five active co-agents. In some embodiments, the plurality of active co-agents consists of, consists essentially of, or comprises from three active co-agents and up to up to six active co-agents. It is understood that co-agent or active co-agent may be used interchangeably, defining a co-agent herein in a combination and/or formulation, which, when in a co-agent combination and/or formulation, said co-agents are synergistic, having synergistic action and causing sufficient and/or effective antimicrobial activity against one or more pathogenic anaerobes (e.g., manifested as any one or more of activities (i) to (x) when provided to one or more pathogenic anaerobes described herein, in which synergistic action and/or antimicrobial activity may be assessed utilizing a standardized in vitro anaerobic AST, which identifies at least a minimum inhibitory concentration (MIC) for a single co-agent or a duo as well as for a co-agent combination described herein tested against a pathogenic anaerobe in an anaerobic environment). In embodiments herein, MIC and calculated fractional inhibitory concentration (FIC) indices (to identify antimicrobial interaction as synergistic (S), additive (A), indifferent (I), or antagonistic (X)) for a three co-agent combination and/or formulation herein against a pathogenic anaerobe are obtained, and MIC and/or FIC indices (FICI) with three co-agents herein is better than an additive activity of each co-agent when provided singly (individually) to said pathogenic anaerobe (utilizing a standardized anaerobic AST). In some embodiments herein, MIC and/or FICI for four co-agents herein against a pathogenic anaerobe herein is better than an additive activity of each co-agent when provided singly (individually) to said pathogenic anaerobe (utilizing a standardized anaerobic AST). In some embodiments herein, MIC and/or FICI for five co-agents herein against a pathogenic anaerobe herein is better than an additive activity of each co-agent when provided singly (individually) to said pathogenic anaerobe (utilizing a standardized anaerobic AST). In some embodiments herein, MIC and/or FICI for six co-agents herein against a pathogenic anaerobe herein is better than an additive activity of each co-agent when provided singly (individually) to said pathogenic anaerobe (utilizing a standardized anaerobic AST). In some embodiments, at least one co-agent is a known antibiotic (currently approved for use as an antibiotic). In some embodiments, at least two co-agents are known antibiotics, each currently approved for use as an antibiotic. In some embodiments, at least three co-agents are known antibiotics, each currently approved as an antibiotic. In some embodiments, at least four co-agents are known antibiotics, each currently approved for use as an antibiotic. In some embodiments, at least five co-agents are known antibiotics, each currently approved for use as an antibiotic. In some embodiments, at least six co-agents are known antibiotics, each currently approved as an antibiotic.

In one or more embodiments, an unpredictable co-agent combination and/or formulation described herein includes at least three co-agents selected from a group consisting of, or consisting essentially of, or comprising: (1) F (as identified above) or an inhibitor of a microbial (bacterial) MurA enzyme (e.g., fosfomycin sodium, fosfomycin tromethamine, fosfomycin trometamol, fosfomycin calcium, fosfomycin sodium, a monobasic or dibasic hydrosoluble fosfomycin salt, and phosphonic derivatives thereof, any having some activity against an aerobic bacteria in aerobic environ, each of which is merely exemplary and representative for purposes herein); (2) D (as identified above) or an inhibitor of a microbial (bacterial) DHFR (e.g., pyrimethamine, trimethoprim, iclaprim, trimetrexate, methotrexate, piritrexim, tetroxoprim, metioprim, brodimoprim, 3,4-diaminopyrimidine, 2,4-diaminopyrimidine, 4-desmethyltrimethoprim, a diaminopyrimidine with at least one R² substituent on a 2,4-diamino-5-aryl-6-substituted pyrimidine derivative, a diaminopyrimidine with at least more hydrophobic groups on a 6-position of 2,4-diaminopyrimidine, a diaminopyrimidine with at least one R² substituent divided into four types: (type 1) at least one R² substituent bearing hydroxy group(s); (type 2) at least one R² substituents bearing alkoxy group(s); (type 3) at least one R² substituent bearing thiazole group(s); (type 4) at least one R² substituent bearing phenyl substituted triazole group(s); each of which is merely exemplary or representative for purposes herein, any having some activity against an aerobic bacteria in aerobic environ); (3) S (as identified above) or an inhibitor of a microbial (bacterial) DHPS (e.g., sulfisoxazole, sulfamethoxazole, sulfadiazine, sulfasalazine, sulfadimethoxine, sulfadoxine, sulfacetamide, zonisamide, para-aminomethylbenzene sulfonamide, sulfacetamide, sulfafurazole, sulfadimidine, sulfaisodimidine, sulfamoxole, sulfanitran, sulfamethoxypyridazine, sulfametoxydiazine, sulfametopyrazine, terephtyl, sulfatroxazole, sulfamerazine, sulfamethazine, sulfapyrazole, sulfaquinoxaline, sulfachloropyridazine, sulfaguanidine, sulfalene, sulfametin, sulfamethoxine, sulfamethylphenazole, sulfaethoxypyridazine, sulfabromomethazine, sulfaphenazole, sulfamoxole, sulfapyrazine, sulfapyridazine, sulfapyridine, sulfasymazine, sulfathiozole, sulfametrole, sulfanilimide, sulfasomidine, each of which is merely exemplary or representative for purposes herein, any having some activity against an aerobic bacteria in aerobic environ); (4) at least one antimicrobial quinazoline (Q) having on its own antimicrobial property (against an aerobic bacteria in aerobic environ), including any suitable phosphate, salt, acid, and/or ester thereof, any of which will inhibit and/or interfere with a microbial (bacterial) DHFR (e.g., SCH79797, irresistin-16, anilinoquinazoline (gefitinib, lapatinib), aminoquinazoline (erlotinib, afatinib), any having some activity against an aerobic bacteria in aerobic environ); (5) an inhibitor of a microbial (bacterial) fosfomycin-modifying enzyme (FMEI), including any suitable phosphate, salt, acid, and/or ester thereof, any of which inhibit or inactivate a fosfomycin-modifying enzyme (FME), such as FosA, FosB, FosC, FosX, or a bacterial metalloenzyme (e.g., glutathione S-transferase, thiol S-transferase, epoxide hydrolase) or a bacterial kinase (e.g., FomA and FomB, functionally related to an FME) (e.g., phosphonoformate, phosphonoacetate, acetylphosphonate, 2-phosphonobutyrate, 4-phosphonobutyrate, 2-phosphonoproprionate, 2-phosphonoproprionate, 3-phosphonoproprionate, methylphosphonate, ethylphosphonate, phenylphosphonate, phosphonoacetaldehyde, sodium phosphonoformate, sodium phosphonoformate tribasic hexahydrate, triethyl phosphonoformate, each of which is merely exemplary or representative for purposes herein); (6) at least one inhibitor of microbial (bacterial) peptidoglycan synthesis (PGSI) having on its own some antimicrobial property (against an aerobic bacteria in aerobic environ), including any suitable phosphate, salt, acid, and/or ester thereof, any of which are in a class of glycopeptide/lipoglycopeptide antibiotics that bind to an outer wall of a bacteria (e.g., to a peptidoglycan) and inhibit or interfere with peptidoglycan synthesis as well as synthesis of the cell wall or bind to enzymes and inhibit cell wall polymerization or crosslinking (e.g., ceftazidime, cefuroxime, cefoxitin, cefoperazone, ceftolozane, cefalexin, cefdinir, ceftriaxone, ceftaroline, cefixime, cefpodoxime, cefazolin, ceftibuten, ceftaroline, cefuroxime, cefotaxime, cefoperazone, cefepime, ceftolazane, ceftriaxone, ceftibiprole, cefiderocol, cefoxitin, moxalactam, S-64922, and the like, including 2^{nd} generation, 3^{rd} generation, 4^{th} generation, 5^{th} generation, and 6^{th} generation), vancomycin, teicoplanin, oritavancin, dalbavancin, telavancin, bleomycin, ramoplanin, decaplanin, diumycin A, coromyin, amoxicillin, meropenem, imipenem, which may be utilized alone or in combination with a BLI (generally as approved for use), in which all examples herein are merely exemplary or representative for purposes herein); (7) an inhibitor of microbial (bacterial) beta lactamase (BLI) (from a class of beta lactamase inhibitors), including any suitable phosphate, salt, acid, and/or ester thereof, utilized alone or more often in combination with an antibiotic, such as with a beta lactam class of antibiotics or other PGSI (e.g., avibactam, clavulanate/clavulanic acid (CA), relebactam, sulbactam, tazobactam, vaborbactam, nacubactam, zidebactam, QPX7728, ETX0282, VNRX5236, alone or in combination with a beta lactam antibiotic (generally as indicated), any of which are merely exemplary or representative for purposes herein).

In some forms, an unpredictable co-agent combination and/or formulation herein, that unexpectedly has synergistic action of co-agents and provides or causes antimicrobial activity (as at least growth inhibitory activity and/or any of activities (i)-(x) herein) against one or more pathogenic anaerobes herein, includes at least the following co-agents: an antimicrobial F (as described herein), an antimicrobial D (as described herein), and an antimicrobial S (as described herein). In some forms, an unpredictable co-agent combination and/or formulation herein, that unexpectedly has synergistic action of co-agents and provides or causes antimicrobial activity (as described herein) against one or more pathogenic anaerobes herein, includes at least the following co-agents: an antimicrobial F, an antimicrobial Q, an antimicrobial S. In some embodiments, Q may replace D, providing yet additional unpredictable co-agent combinations and/or formulations that unexpectedly have synergistic action of co-agents and provide or cause antimicrobial activity (as described herein) against one or more pathogenic anaerobes herein. In some forms, an unpredictable co-agent combination and/or formulation herein, that unexpectedly has synergistic action of co-agents and provides or causes antimicrobial activity (as described herein) against one or more pathogenic anaerobes herein, includes at least co-agents: F, D, S, and PGSI. In some forms, an unpredictable co-agent combination and/or formulation herein, that unexpectedly has synergistic action of co-agents and provides or causes antimicrobial activity (as described herein) against one or more pathogenic anaerobes herein, includes at least co-agents: F, D, S, and FMEI. In some forms, an unpredictable co-agent combination and/or formulation herein, that unexpectedly has synergistic action of co-agents and provides or causes antimicrobial activity (as described herein) against one or more pathogenic anaerobes herein includes at least co-agents: F, D, S, and BLI, when the BLI itself possesses antimicrobial activity.

In one or more embodiments, an unpredictable co-agent combination and/or formulation herein, with unexpected synergistic action of co-agents, provides or causes antimicrobial activity (as at least growth inhibitory activity and/or any of activities (i)-(x) as described herein) against one or more pathogenic anaerobes herein, in which co-agents are or include: an inhibitor of a bacterial MurA enzyme having some antibacterial property; an inhibitor of bacterial DHFR having some antibacterial property; and an inhibitor of DHPS having some antibacterial property. Optionally, co-agents also include PGSI. Optionally, co-agents also include a BLI, when the BLI itself possess antimicrobial activity. Optionally, co-agents also include a FMEI. Optionally, co-agents also include a PGSI and a BLI. Optionally, co-agents also include a BLI, and a beta-lactam antibiotic (a PGSI).

Any co-agent combinations and/or formulations herein target one or more pathogenic anaerobes, the co-agents engaging in synergistic action as synergists, promoting or causing sufficient and/or effective antimicrobial activity against one or more pathogenic anaerobes herein (initiating at least one of activities (i)-(x) described herein and/or bacterial growth inhibitory activity and/or bacterial interference or elimination), in which a targeted anaerobe is at least a type and/or species of pathogenic anaerobe, including but is not limited to, an obligate anaerobe, obligate anaerobe in a mixed population, GPOA, GNOA, GPOA in a mixed population, GNOA in a mixed population, drug-resistant obligate anaerobe (intrinsic and/or acquired resistance), and any combination or arrangement thereof. In some embodiments, a targeted anaerobe is in an anaerobic environment. In some embodiments, a targeted anaerobe is in a pH neutral environment. In some embodiments, a targeted anaerobe is in a basic pH environment (e.g., above a neutral pH and/or to about pH 8 or to about pH 9, or in any pH range therebetween). In some embodiments, a targeted anaerobe is in an acidic pH environment (e.g., below a neutral pH and/or to about pH 5 or to about pH 6, or in any pH range therebetween). Unexpectedly, it has been found that co-agent combinations and/or formulations herein effectively target one or more pathogenic anaerobes, the co-agents undergoing synergistic action as synergists, promoting or causing sufficient and/or effective antimicrobial activity (as described herein) against one or more pathogenic anaerobes herein, in any or all of the following: (aa) neutral pH; (bb) acidic pH; and (cc) basic pH.

To cause or initiate synergistic action and synergism of co-agents, which promotes or causes sufficient and/or effective antimicrobial activity against one or more pathogenic anaerobes herein (as at least one of activities (i)-(x) herein and/or bacterial growth inhibitory activity and/or bacterial interference or elimination of at least colony formation), three or more co-agents herein are delivered or dispensed (or administered or conveyed or presented) in a suitable manner so that said synergistic action and synergism of co-agents occur. In some embodiments, a delivering or dispensing (or administering or conveying or presenting) is a co-dosing of three or more co-agents, co-dosing occurring at or about a same time, so as to garner synergism, synergistic action, and sufficient and/or effective antimicrobial activity. In some embodiments, a delivering or dispensing (or administering or conveying or presenting) is a co-dosing of three or more co-agents, co-dosing occurring in a period, so as to garner synergism, synergistic action, and sufficient and/or effective antimicrobial activity. In some embodiments, a delivering or dispensing (or administering or conveying or presenting) is a co-dosing of three or more co-agents, co-dosing occurring in an overlapping period, so as to garner synergism, synergistic action, and sufficient and/or effective antimicrobial activity. The three or more co-agents may be provided in whole or in one or more parts (e.g., doses), each delivery or dispensing (or administering or conveying or presenting) being independent or continuous, and co-agents being provided concurrently or in a progression or in an acceptable period. The three or more co-agents may be in a same vehicle, or in different vehicles, or in two or more vehicles, or in three or more vehicles, or in four or more vehicles. In one or more embodiments, to cause or initiate synergistic action by active co-agents and promote or cause sufficient and/or effective antimicrobial activity (as herein described) against one or more pathogenic anaerobes herein, three or more co-agents herein are provided or maintained or supported in or by at least one vehicle, which may also be for delivering or dispensing or administering or conveying or presenting co-agents), or may be followed by the delivering or dispensing or administering or conveying or presenting co-agents (concurrently or in progression, in one or more vehicles) at about a same period or a substantially same period. In one or more embodiments, to cause or initiate synergistic action by active co-agents and promote or cause sufficient and/or effective antimicrobial activity (as herein described) against one or more pathogenic anaerobes herein, three or more co-agents herein are provided or maintained or supported in or by at least one vehicle, which may also be for delivering or dispensing or administering or conveying or presenting co-agents), or may be followed by the delivering or dispensing or administering or conveying or presenting co-agents (concurrently or in progression, in one or more vehicles) in a period or overlapping period, the period being any of a few minutes, about or up to about 15 min, about or up to about 30 min, about or up to about 1 hour, about or up to about 2 hours, about or up to about 3 hours, or any period or any range therebetween.

In some embodiments, use of three or more co-agents in a co-agent combination and/or formulation herein as pharmacotherapy or a medicament provides a new use for at least one co-agent or more than one co-agent. In some embodiments, use of three or more co-agents in a co-agent combination and/or formulation herein as pharmacotherapy or a medicament revives at least one co-agent or more than one co-agent. In some embodiments, use of three or more co-agents in a co-agent combination and/or formulation herein as pharmacotherapy or a medicament provides enhanced therapeutic potential (or potency) for at least one co-agent or more than one co-agent. In some embodiments, use of three or more co-agents in a co-agent combination and/or formulation herein as pharmacotherapy or a medicament reverses inactivity of at least one of the co-agents or more than one co-agent being on its own inactive against one or more pathogenic anaerobes herein.

In embodiments herein, a delivering or dispensing (or administering or conveying or presenting) of active co-agents, in a manner herein or above, will initiate or cause or effect a response including any one or more of: (a) reduction in growth of one or more pathogenic anaerobes (or growth reduction); (b) inhibition of growth of one or more pathogenic anaerobes (or growth inhibition); (c) suspension of growth of the one or more pathogenic anaerobe (or growth suspension); (d) elimination in whole or in part of one or more pathogenic anaerobes (or microbial elimination); (e) reduction in whole or in part of one or more pathogenic anaerobes (or microbial reduction); (f) interference in whole or in part of one or more pathogenic anaerobes (or microbial interference); (g) altering and/or enhancing and/or promoting susceptibility of one or more pathogenic anaerobes (that may or may not include one or more previously non-susceptible pathogenic anaerobe and/or one or more intrinsically resistant pathogenic microorganisms), via synergism of active co-agents, in which one or more active co-agent is a synergist, promoting or causing synergistic action, for sufficient and/or effective antimicrobial activity against one or more pathogenic anaerobes (which may include ones now susceptible to the active combination and/or formulation herein described while not susceptible to one or more co-agents when used singly or as a duo). In some embodiments, pathogenic anaerobes are or include an obligate anaerobe (GPOA and/or GNOA) in an anaerobic environment.

In some embodiments, when a co-agent composition and/or formulation herein (comprising a plurality of co-agents or three or more active co-agents herein) is provided as pharmacotherapy to a host or subject having or suspected of having one or more pathogenic anaerobes herein or an infection caused or suspected of being caused by the one or more pathogenic anaerobes (e.g., in need thereof), co-agents initiate or cause a synergistic action and a sufficient and/or effective antimicrobial activity against one or more susceptible pathogenic anaerobes, in which one or more of responses (a) to (g) arise. Any one or more responses (a) to (g) will occur, generally, in a matter of one or more days to a few weeks, after providing the plurality of co-agents via at least one means of delivery or dispensing. In many embodiments, the providing of the plurality of co-agents to the host or subject in need thereof is daily, by a delivery or dispensing means, via one or more vehicles (containing one or more co-agents), in which the plurality of co-agents are co-dosed in a period (the co-agents being in one or more formulations, and contained in one or more vehicles for delivery and/or dispensing). Co-dosing of one or more formulations in a period via a delivery or dispensing of one or more vehicles in a period allows association and mingling of co-agents, so as to initiate or cause synergistic action and sufficient and/or effective antimicrobial activity against the one or more pathogenic anaerobes (such as in an anaerobic environment). In some embodiments, the providing of the plurality of co-agents is for therapy or treatment of a host or subject having or suspected of having at least one pathogenic anaerobe considered or found resistant (acquired resistance or intrinsic resistance) to one or more antibiotic, in which the plurality of co-agents is active against the at least one pathogenic anaerobe considered or found resistant to the antibiotic, so as to overcome or reverse the resistance. In some embodiments, the providing of the plurality of co-agents is for therapy or treatment of a host or subject having or suspected of having at least one pathogenic anaerobe considered or found resistant (acquired resistance or intrinsic resistance) to at least one co-agent (of the plurality of co-agents), in which the plurality of co-agents is active against the at least one pathogenic anaerobe considered or found resistant to the at least one co-agent, so as to overcome or reverse the resistance. The resistance may be evaluated via an established in vitro AST for anaerobes. In many embodiments, the one or more susceptible pathogenic anaerobes or the at least one pathogen (having acquired or intrinsic resistance) in or on the host or subject (or suspected thereof) is an obligate anaerobe in an anaerobic environment. The obligate anaerobe may be in a mixed population. In many embodiments, the one or more susceptible pathogenic anaerobes or the at least one pathogen (having acquired resistance or intrinsic resistance) in or on the host or subject (or suspected thereof) is a GPOA and/or GNOA and is not a non-responsive facultative anaerobe selected from one or more of Lactobacillus spp., Leuconostoc spp., and/or Pediococcus spp. The one or more susceptible pathogenic anaerobes or the at least one pathogen (having acquired or intrinsic resistance) in or on the host or subject (or suspected thereof) is or may include one or more or a combination of a GPOA, a GNOA, a Gram-positive facultative anaerobe, a Gram-negative facultative anaerobe, any of which may or may not be resistant to at least one co-agent (individually) by acquired or intrinsic resistance. In some embodiments, the one or more susceptible pathogenic anaerobes or the at least one pathogen (having acquired or intrinsic resistance) is in an anaerobic environment. The environment may be at a neutral pH, or an acidic pH (below 7 and as low as 5 or as low as 6), or a basic pH (above 7 and as high as 8 or as high as 9) without significantly altering co-agent synergy. The three or more co-agents may be in one form and/or formulation, each being in a therapeutically effective amount, the co-agents being at least F, D, S, or F, Q, S. The three or more co-agents may be in one or more separate forms and/or formulations, each being in a therapeutically effective amount, the co-agents being at least F, D, S, or F, Q, S. The three or more co-agents may be in one form and/or formulation, each being in a therapeutically effective amount, the co-agents being at least F, D, S, FMEI or F, Q, S, FMEI. The three or more co-agents may be in one or more separate forms and/or formulations, each being in a therapeutically effective amount, the co-agents being at least F, D, S, FMEI or F, Q, S, FMEI. The three or more co-agents may be in one form and/or formulation, each being in a therapeutically effective amount, the co-agents being at least F, D, S, PGSI or F, Q, S, PGSI. The three or more co-agents may be in one or more separate forms and/or formulations, each being in a therapeutically effective amount, the co-agents being at least F, D, S, PGSI or F, Q, S, PGSI. The three or more co-agents may be in one form and/or formulation, each being in a therapeutically effective amount, the co-agents being at least F, D, S, BLI or F, Q, S, BLI, when BLI itself possess antimicrobial activity. The three or more co-agents may be in one or more separate forms and/or formulations, each being in a therapeutically effective amount, the co-agents being at least F, D, S, BLI or F, Q, S, BLI, when BLI itself possess antimicrobial activity. The three or more co-agents may be in one form and/or formulation, each being in a therapeutically effective amount, the co-agents being at least F, D, S, PGSI, BLI or F, Q, S, PGSI, BLI (generally when the PGSI is from a class of beta lactam antibiotics). The three or more co-agents may be in one or more separate forms and/or formulations, each being in a therapeutically effective amount, the co-agents being at least F, D, S, PGSI, BLI or F, Q, S, PGSI, BLI (generally when the PGSI is from a class of beta lactam antibiotics). The three or more co-agents may be in one form and/or formulation or in more than one separate forms and/or formulations, each co-agent being in a therapeutically effective amount, the co-agents being at least: an inhibitor of a bacterial MurA enzyme and having some antibacterial property in aerobic environ; an inhibitor of bacterial DHFR and having some antibacterial property in aerobic environ; and an inhibitor of bacterial DHPS and having some antibacterial property in aerobic environ. Q, as an inhibitor of bacterial DHFR having some antibacterial property in aerobic environ, when in a therapeutically effective amount, may be substituted for D (or for an inhibitor of a bacterial DHFR herein), in which substitution will initiate or cause synergistic action and sufficient and/or effective antimicrobial activity against the one or more susceptible pathogenic anaerobes or the at least one pathogen (having acquired or intrinsic resistance) in an anaerobic environment.

When a co-agent combination and/or formulation comprises F, D, S or F, Q, S, a further co-agent may be provided, in which the further co-agent is at least a PGSI, and the pathogenic anaerobe is or includes at least a GPOA and/or GNOA. In some embodiments, a further co-agent is at least a BLI, when the BLI possesses antimicrobial activity, and the pathogenic anaerobe is or includes at least a GPOA and/or GNOA. In some embodiments, a further co-agent is at least a FMEI, and the pathogenic anaerobe is or includes at least a GPOA and/or GNOA. In some embodiments, a further co-agent is at least a PGSI (as a beta lactam antibiotic) and a BLI, and the pathogenic anaerobe is or includes at least a GPOA and/or a GNOA. In some forms, the further co-agent is amoxicillin and clavulanate, and the pathogenic anaerobe is or includes at least a GPOA and/or GNOA. In some forms, the further co-agent is vancomycin, and the at least one pathogenic anaerobe is or includes at least a GPOA and/or GNOA.

In some embodiments is a method of utilizing an effective amount of a fosfomycin or an inhibitor of a microbial (bacterial) MurA enzyme (comprising a stereoisomer thereof, geometric isomer thereof, tautomer thereof, hydrate thereof, solvate thereof, pharmaceutically acceptable phosphate, salt, acid, amine, and/or ester thereof, and any combination thereof having activity against an aerobic bacteria), as a synergist or active co-agent for an antibiotic combination, the antibiotic combination comprising: (1) a diaminopyrimidine or an inhibitor of a microbial (bacterial) DHFR, and (2) a sulfonamide or an inhibitor of a microbial (bacterial) DHPS, the method comprising: providing to a host or subject in need thereof an effective amount and/or sufficient amount of the antibiotic combination; and including an effective amount and/or sufficient amount of the synergist or active co-agent, wherein the antibiotic combination and the synergist or active co-agent are provided together or in a period of time comprising within about one hour or two hours or three hours, wherein the host or subject has or is suspected of having an infection from one or more type and/or species of obligate anaerobic bacteria being one type and/or species or different types and/or species, wherein the diaminopyrimidine or inhibitor of the microbial (bacterial) DHFR is a stereoisomer, geometric isomer, a tautomer, a hydrate, a solvate, a pharmaceutically acceptable phosphate, salt, acid, and/or ester thereof, and any combination thereof, and wherein the sulfonamide or inhibitor of the microbial (bacterial) DHPS is a stereoisomer, geometric isomer, a tautomer, a hydrate, a solvate, a pharmaceutically acceptable phosphate, salt, acid, and/or ester thereof, and any combination thereof, and wherein the diaminopyrimidine and the sulfonamide each alone/singly have some antibacterial property against an aerobic bacteria in an aerobic environment.

In some embodiments is a method of utilizing an effective amount of an inhibitor of a microbial (bacterial) peptidoglycan synthesis (PGSI) (comprising a stereoisomer thereof, geometric isomer thereof, tautomer thereof, hydrate thereof, solvate thereof, pharmaceutically acceptable phosphate salt, acid, and/or ester thereof, and any combination thereof), as a synergist or active co-agent for an antibiotic combination, the antibiotic combination comprising: (1) a fosfomycin or an inhibitor of a microbial (bacterial) MurA enzyme; (2) a diaminopyrimidine or an inhibitor of a microbial (bacterial) DHFR, and (3) a sulfonamide or an inhibitor of a microbial (bacterial) DHPS, each when alone have some antibacterial property against an aerobic bacteria; the method comprising: providing to a host or subject in need thereof an effective and/or sufficient amount of the antibiotic combination; and including an effective amount and/or sufficient amount of the synergist or active co-agent, wherein the antibiotic combination and the synergist or active co-agent are provided together or in a period comprising within about one hour or two hours or three hours, wherein the host or subject has or is suspected of having an infection from one or more type and/or species of anaerobic bacteria, the anaerobic bacteria being one type and/or species or different types and/or species, wherein the diaminopyrimidine or inhibitor of the microbial (bacterial) DHFR is a stereoisomer thereof, geometric isomer, a tautomer, a hydrate, a solvate thereof, a pharmaceutically acceptable phosphate, salt, acid, and/or ester thereof, and any combination thereof, wherein the sulfonamide or inhibitor of the microbial (bacterial) DHPS is a stereoisomer, geometric isomer, a tautomer, a hydrate, a solvate thereof, a pharmaceutically acceptable phosphate, salt, acid, and/or ester thereof, and any combination thereof, and wherein the fosfomycin or inhibitor of microbial (bacterial) MurA enzyme is a stereoisomer, geometric isomer, a tautomer, a hydrate, a solvate thereof, a pharmaceutically acceptable phosphate, salt, acid, amine, and/or ester thereof, and any combination thereof.

In some embodiments is a method of utilizing an effective amount of an inhibitor of a microbial (bacterial) fosfomycin-modifying enzyme (FMEI) (comprising a stereoisomer thereof, geometric isomer thereof, tautomer thereof, hydrate thereof, solvate thereof, pharmaceutically acceptable phosphate, salt, acid, and/or ester thereof, and any combination thereof), as a synergist or active co-agent for an antibiotic combination, the antibiotic combination comprising: (1) a fosfomycin or an inhibitor of a microbial (bacterial) MurA enzyme; (2) a diaminopyrimidine or an inhibitor of a microbial (bacterial) DHFR; (3) and a sulfonamide or an inhibitor of a microbial (bacterial) DHPS, each when alone have some antibacterial property against an aerobic bacteria; the method comprising: providing to a host or subject in need an effective and/or sufficient amount of the antibiotic combination; and including an effective amount and/or sufficient amount of the synergist or active co-agent, wherein the antibiotic combination and the synergist or active co-agent are provided together or in a period comprising within about one hour or two hours or three hours, wherein the host or subject has or is suspected of having an infection from one or more type and/or species of anaerobic bacteria, the anaerobic bacteria being one or different types and/or species, wherein the diaminopyrimidine or inhibitor of the microbial (bacterial) DHFR is a stereoisomer thereof, geometric isomer thereof, tautomer thereof, a hydrate thereof, a solvate thereof, a pharmaceutically acceptable phosphate, salt, acid, and/or ester thereof, and any combination thereof, wherein the sulfonamide or inhibitor of the microbial (bacterial) DHPS is a stereoisomer thereof, geometric isomer thereof, a tautomer thereof, a hydrate thereof, a solvate thereof, a pharmaceutically acceptable phosphate, salt, acid, and/or ester thereof, and any combination thereof, and wherein the fosfomycin or inhibitor of microbial (bacterial) MurA enzyme is a stereoisomer thereof, geometric isomer thereof, a tautomer thereof, a hydrate thereof, a solvate thereof, a pharmaceutically acceptable phosphate, salt, acid, amine and/or ester thereof, and any combination thereof.

In some embodiments is a method of utilizing an effective amount of an inhibitor of a microbial (bacterial) beta lactamase (BLI) (comprising a stereoisomer, geometric isomer, tautomer, hydrate, solvate, pharmaceutically acceptable phosphate, salt, acid, and/or ester thereof, and any combination thereof, when the BLI, itself, has antibacterial property), as a synergist or active co-agent for an antibiotic combination, the antibiotic combination comprising: (1) a fosfomycin or an inhibitor of a microbial (bacterial) MurA enzyme; (2) a diaminopyrimidine or an inhibitor of a microbial (bacterial) DHFR, and (3) a sulfonamide or an inhibitor of a microbial (bacterial) DHPS, each when used alone have some antibacterial property against an aerobic bacteria; the method comprising: providing to a host or subject in need thereof an effective and/or sufficient amount of the antibiotic combination; and including an effective amount and/or sufficient amount of the synergist or active co-agent, wherein the antibiotic combination and the synergist or active co-agent are co-dosed, being provided together or in a period comprising within or about one hour or two hours or three hours, wherein the host or subject has or is suspected of having an infection from one or more type and/or species of anaerobic bacteria, the anaerobic bacteria being one or different types and/or species, wherein the diaminopyrimidine or inhibitor of the microbial (bacterial) DHFR is a stereoisomer, geometric isomer, tautomer, hydrate, solvate, pharmaceutically acceptable phosphate, salt, acid, and/or ester thereof, and any combination thereof, wherein the sulfonamide or inhibitor of the microbial (bacterial) DHPS is a stereoisomer, geometric isomer, tautomer, hydrate, solvate thereof, pharmaceutically acceptable phosphate, salt, acid, and/or ester thereof, and any combination thereof, and wherein the fosfomycin or inhibitor of microbial (bacterial) MurA enzyme is a stereoisomer thereof, geometric isomer thereof, tautomer thereof, hydrate thereof, solvate thereof, pharmaceutically acceptable phosphate, salt, acid, amine, and/or ester thereof, and any combination thereof.

In one or more methods herein, the steps of providing and including promote or initiate or cause a synergistic action of co-agents as synergists. The steps of providing and including and due to a co-dosing of synergist or co-agent and antibiotic combination or co-agents within a period promote or initiate or cause a synergistic enhancement of activity of the antibiotic combination or co-agents as pharmacotherapy against one or more pathogenic anaerobe. The steps of providing and including may involve a co-dosing of all co-agents and synergists in a period, which may be one period up to twelve periods daily until appropriate to discontinue. The steps of providing and including occur together or substantially together or during a period, the period comprising within or less than about one hour or within or less than about two hours or within or less than three hours or continuous. The steps of providing and including may lead to or bring about or induce sufficient and/or effective antimicrobial activity of the combination or antibiotic combination comprising co-agents and/or synergists, against one or more type and/or species of anaerobic bacteria. In one or more methods herein, the step of further including occurs together or substantially together or during a same period, with the step of providing and/or the step of including, the period comprising within or less than about one hour or within or less than about two hours or within or less than three hours. In addition, or as an alternative, the steps of providing and including a combination or antibiotic combination comprising co-agents and/or synergists and/or further co-agents and/or further synergists may occur in one of a neutral pH environment, an acidic pH environment, or a basic pH environment, without disturbing or disrupting synergistic action, and/or sufficient and/or effective antimicrobial activity.

Herein is also a plurality of co-agents for utilization as an antimicrobial medicament against one or more pathogenic anaerobes in an anaerobic environment, the plurality of co-agents comprising: a fosfomycin in a pharmaceutically acceptable form selected from one or more of a salt, phosphate, acid, amine, and ester, in which the fosfomycin alone is active against an aerobic bacteria in an aerobic environment, and may be substituted for an inhibitor of bacterial UDP-GlcNAc enolpyruvyl transferase that has some activity against an aerobic bacteria in an aerobic environment; a diaminopyrimidine in a pharmaceutically acceptable form selected from one or more of a salt, phosphate, acid, and ester, in which the diaminopyrimidine is active against an aerobic bacteria in an aerobic environment, and may be substituted for an inhibitor of bacterial dihydrofolate reductase that has some activity against an aerobic bacteria in an aerobic environment; and a sulfonamide in a pharmaceutically acceptable form selected from one or more of a salt, phosphate, acid, and ester, in which the sulfonamide is active against an aerobic bacteria in an aerobic environment, and may be substituted with an inhibitor bacterial dihydropteroate synthase that has some activity against an aerobic bacteria in an aerobic environment, wherein the plurality of co-agents are provided in at least one period to a subject in need thereof causing one or more of the plurality of co-agents to act as a synergist and causing antibacterial activity against the one or more pathogenic anaerobes in the anaerobic environment, the at least one period being selected from a group consisting of about three hours or less, about two hours or less, and about 1 hour or less. The plurality of co-agents may be active against at least one of the one or more pathogenic anaerobes when the at least one of the one or more pathogenic anaerobes is in any of an acidic pH, a neutral pH, and a basic pH, wherein the acidic pH is a pH as low as about pH 5, and wherein the basic pH is a pH as high as about pH 8. At least one of the one or more pathogenic anaerobes may be resistant to at least one co-agent of the plurality of co-agents when that at least one co-agent is used singly against the at least one of the one or more pathogenic anaerobes, and wherein at least one of the one or more pathogenic anaerobes is an obligate anaerobic bacteria. The plurality of co-agents may be provided as pharmacotherapy to the subject, the plurality of co-agents being provided by co-dosing in the period, wherein the co-dosing is further selected from a group consisting of at a same time, at substantially a same time, in an overlapping expanse of time within the period, and in a series within the period, and wherein, in 24 hours, the co-dosing in the period is selected from one of the group consisting of once, twice, three times, four times, six times, eight times, twelve times, and continuously. The plurality of co-agents may be provided as pharmacotherapy to the subject having or suspected of having an infection caused by or suspected of being caused by one or more Gram-positive obligate anaerobic bacteria in an anaerobic environment in or on the subject. The plurality of co-agents may be provided as pharmacotherapy to the subject having or suspected of having an infection caused by or suspected of being caused by one or more Gram-negative obligate anaerobic bacteria in an anaerobic environment in or on the subject. The plurality of co-agents may be provided as pharmacotherapy to the subject having or suspected of having an infection caused by or suspected of being caused by one or more challenging anaerobes selected from a microaerophilic Streptococcus, including one or more of S. anginosus, S. constellatus, S. intermedius, S. mutans, and viridans streptococci, and the plurality of co-agents is active against the one or more microaerophilic Streptococcus. The plurality of co-agents may be provided as pharmacotherapy to the subject having or suspected of having an infection caused by or suspected of being caused by one or more Gram-positive obligate anaerobic bacteria selected from one or more in a group consisting of Actinomyces spp., Arcanobacterium spp., Atopobium spp., Bifidobacterium spp., Bilophila spp., Clostridioides spp., Clostridium spp., Collinsella spp., Eggerthella spp., Eubacterium spp., Finegoldia spp., Parvimonas spp., Peptococcus spp., Peptostreptococcus spp., Propionibacterium spp., and Cutibacterium spp., and the plurality of co-agents are active against the one or more one or more Gram-positive obligate anaerobic bacteria. The plurality of co-agents may be provided as pharmacotherapy to the subject in need thereof, the subject having or suspected of having an infection caused by or suspected of being caused by one or more Gram-negative obligate anaerobic bacteria selected from one or more in a group consisting of Aggretibacter spp., Bacteroides spp., Parabacteroides spp., Dethiosulfovibrio spp., Fusobacterium spp., Phocaeicola spp., Porphyromonas spp., Prevotella spp., Sutterella spp., Veillonella spp., and the plurality of co-agents is active against the one or more one or more Gram-negative obligate anaerobic bacteria. The plurality of co-agents, when provided as pharmacotherapy to the subject, are not active or effective against one or more commensal anaerobic bacteria species selected from a group consisting of Lactobacillus spp., Pediococcus spp., and Leuconostoc spp.

In other embodiments, is a synergist for an antibiotic combination, the antibiotic combination comprising: a diaminopyrimidine in a pharmaceutically acceptable form selected from one or more of a salt, phosphate, acid, and ester, in which the diaminopyrimidine may be substituted for an inhibitor of bacterial dihydrofolate reductase, any of which will when used singly have: (a) some activity against an aerobic bacteria in an aerobic environment; and (b) no activity against one or more obligate anaerobic bacteria in an anaerobic environment; and a sulfonamide in a pharmaceutically acceptable form selected from one or more of a salt, phosphate, acid, and ester, in which the sulfonamide may be substituted for an inhibitor of bacterial dihydropteroate synthase, any of which will when used singly have: (a) some activity against an aerobic bacteria in an aerobic environment; and (b) no activity against one or more obligate anaerobic bacteria in an anaerobic environment; wherein the diaminopyrimidine and the inhibitor of bacterial dihydrofolate reductase will inhibit or inactive a dihydrofolate reductase enzyme of the one or more obligate anaerobic bacteria, and wherein the sulfonamide and the inhibitor of bacterial dihydropteroate synthase will inhibit or inactive a dihydropteroate synthase enzyme of the one or more obligate anaerobic bacteria, and wherein the synergist comprises at least: a fosfomycin in a pharmaceutically acceptable form selected from one or more of a salt, phosphate, amine, acid, and ester, in which the fosfomycin may be substituted for an inhibitor of bacterial UDP-GlcNAc enolpyruvyl transferase (MurA), any of which will when used singly have: (a) some activity against an aerobic bacteria in an aerobic environment; and (b) no activity against one or more obligate anaerobic bacteria in an anaerobic environment; wherein the synergist with the antibiotic combination causes synergistic action so as to transform therapeutic potential of the antibiotic combination, so that the synergist with the antibiotic combination are active against the one or more obligate anaerobic bacteria in the anaerobic environment. The synergist and the antibiotic combination may be in a same formulation or different formulations, and for synergistic action, the synergist and antibiotic combination are provided in a same period, the period being selected from one of a group consisting of within or less than about one hour, within or less than about two hours, and within or less then about three hours. The synergist and the antibiotic combination, as pharmacotherapy for a subject in need thereof, are provided to the subject in a period, the period for providing the synergist and the antibiotic combination being selected from one of a group consisting of a same period, a substantially same period, an overlapping expanse of time within the period, and in a series within the period. The fosfomycin or inhibitor of bacterial MurA may be selected from one or more of a group consisting of a phosphonate, a phosphonic acid, a derivative of phosphonate, a derivative of phosphonic acid, a stereoisomer thereof, a geometric isomer thereof, a tautomer thereof, a hydrate thereof, a solvate thereof, and wherein a representative example of a fosfomycin is a hydrosoluble fosfomycin salt. The diaminopyrimidine or inhibitor of bacterial dihydrofolate reductase may be selected from one or more of a group consisting of a 2,4-diaminopyrimidine, a derivative of 2,4-diaminopyrimidine, a stereoisomer thereof, a geometric isomer thereof, a tautomer thereof, a hydrate thereof, a solvate thereof, and wherein representative examples of a diaminopyrimidine are selected from one or more of trimethoprim, pyrimethamine, diaveridine, brodimoprim, tetroxoprim, metioprim, and iclaprim. The sulfonamide or inhibitor of bacterial dihydropteroate synthase may be selected from one or more of a group consisting of a sulfanilamide, a derivative of sulfanilamide, a sulfam, a derivative of a sulfam, a sulfonamide, a derivative of a sulfonamide, a disulfonimide, a derivative of a disulfonimide, a stereoisomer thereof, a geometric isomer thereof, a tautomer thereof, a hydrate thereof, a solvate thereof, and wherein representative examples of a sulfonamide are selected from one or more of sulfadiazine, sulfamethoxazole, sulfatroxazole, sulfamerazine, sulfadoxine, sulfadimethoxine, sulfamethazine, sulfapyrazole, sulfaquinoxaline, sulfachloropyridazine, sulfaguanidine, sulfalene, sulfametin, sulfamethoxine, sulfamethoxy-pyridazine, sulfamethylphenazole, sulfamethoxypyridazine, sulfaethoxypyridazine, sulfabromomethazine, sulfaphenazole, sulfamoxole, sulfapyrazine, sulfapyridazine, sulfapyridine, sulfasymazine, sulfathiozole, sulfametrole, sulfanilimide, sulfasomidine, and sulfisoxazole. The synergist may further comprise a co-agent, the co-agent being an inhibitor of a bacterial fosfomycin modifying (FME) enzyme selected from one or more of a group consisting of a phosphonoformate, a phosphonoacetate, a methylphosphonate, an ethylphosphonate, a phenylphosphonate, an acetylphosphonate, a phosphonoacetaldehyde, a stereoisomer thereof, a geometric isomer thereof, a tautomer thereof, a hydrate thereof, a solvate thereof, and wherein representative examples are selected from one or more of sodium phosphonoformate, sodium phosphonoformate tribasic hexahydrate, triethyl phosphonoformate, 2-phosphonobutyrate, 4-phosphonobutyrate, 2-phosphonoproprionate, 2-phosphonoproprionate, and 3-phosphonoproprionate, and wherein the inhibitor of the bacterial FME will inhibit or inactive FME of the one or more obligate anaerobic bacteria. The synergist may further comprises a co-agent, the co-agent being one or more from a group of co-agents consisting of: (a) an inhibitor of bacterial peptidoglycan synthesis selected from a glycopeptide or lipoglycopeptide class of antibiotics, wherein inhibitor of bacterial peptidoglycan synthesis will bind to a lipid II precursor of an outer wall of the one or more obligate anaerobic bacteria, inhibiting peptidoglycan synthesis and inhibiting synthesis of a cell wall of the one or more obligate anaerobic bacteria; (b) a beta lactam antibiotic selected from a class of beta lactam antibiotics, wherein the beta lactam antibiotic will bind to a penicillin-binding protein enzyme and inhibit peptidoglycan synthesis of the one or more obligate anaerobic bacteria; and (c) an inhibitor of bacterial beta-lactamase, wherein the inhibitor of bacterial beta-lactamase will inhibit or inactive a beta-lactamase enzyme of the one or more obligate anaerobic bacteria, and wherein the co-agent is provided in a same period as the synergist and the antibiotic combination, the period being selected from one of a group consisting of within or less than about one hour, within or less than about two hours, and within or less then about three hours, and wherein the synergist and the antibiotic combination and the co-agent are, in 24 hours, provided for one of the group consisting of once, twice, three times, four times, six times, eight times, twelve times, and continuously.

Also herein is a kit containing co-agents for utilization as an antimicrobial against one or more pathogenic anaerobes in an anaerobic environment, the kit comprising at least: a first co-agent in a therapeutically effective amount for delivery of the first co-agent in 24 hours, the first co-agent being a fosfomycin in a pharmaceutically acceptable form selected from one or more of a salt, phosphate, acid, amine, and ester, wherein the first co-agent when used alone in an aerobic environment is an inhibitor of bacterial UDP-GlcNAc enolpyruvyl transferase; a second co-agent in a therapeutically effective amount for delivery of the second co-agent in 24 hours, the second co-agent being a diaminopyrimidine in a pharmaceutically acceptable form selected from one or more of a salt, phosphate, acid, and ester, wherein the second co-agent when used alone in an aerobic environment is an inhibitor of bacterial dihydrofolate reductase; a third co-agent in a therapeutically effective amount for delivery of the third co-agent in 24 hours, the third co-agent being a sulfonamide in a pharmaceutically acceptable form selected from one or more of a salt, phosphate, acid, and ester, wherein the second co-agent when used alone in an aerobic environment is an inhibitor bacterial dihydropteroate synthase; and optionally at least one diluent for any one or more of the first co-agent, the second co-agent, and the third co-agent, wherein the first co-agent, the second co-agent and the third co-agent are in one or more vehicles, the one or more vehicles comprising a sufficient amount for at least one delivery in the 24 hours, wherein the kit is for delivery of a therapeutically effective amount of the first co-agent and a therapeutically effective amount of the second co-agent and therapeutically effective amount of the third co-agent for at least one delivery to a subject for treating or preventing an infection from one or more obligate anaerobic bacteria.

Herein is also synergist or co-agent or active co-agent for an antibiotic combination comprising: at least an effective amount or therapeutically effective amount of a diaminopyrimidine or an inhibitor of a microbial (bacterial) DHFR; and an effective amount or therapeutically effective amount of a sulfonamide or an inhibitor of a microbial (bacterial) DHPS, each when alone have some antibacterial property against an aerobic bacteria; the synergist or co-agent or active co-agent comprising an effective amount or therapeutically effective amount of a fosfomycin or an inhibitor of a microbial (bacterial) MurA enzyme (comprising a stereoisomer, a geometric isomer, a tautomer, a hydrate, a solvate, a pharmaceutically acceptable phosphate, salt, acid, amine, and/or ester thereof, and combinations thereof, that has some antibacterial property against an aerobic bacteria), wherein the synergist or co-agent or active co-agent for the antibiotic combination initiates or causes or imparts synergistic action and an effective and/or sufficient antimicrobial activity against one or more pathogenic anaerobes, wherein the antibiotic combination and the synergist or co-agent or active co-agent are in a same formulation or different formulation, wherein the antibiotic combination and the synergist or co-agent or active co-agent are provided at a same period or substantially a same period, the period being within or less than about one hour or within or less than about two hours or within or less than about three hours, wherein the diaminopyrimidine or inhibitor of microbial (bacterial) DHFR is a stereoisomer thereof, geometric isomer thereof, a tautomer thereof, a hydrate thereof, a solvate thereof, a pharmaceutically acceptable phosphate, salt, acid, and/or ester thereof, and combinations thereof, and wherein the sulfonamide or inhibitor of the microbial (bacterial) DHPS is a stereoisomer thereof, geometric isomer thereof, a tautomer thereof, a hydrate thereof, a solvate thereof, a pharmaceutically acceptable phosphate, salt, acid, and/or ester thereof, and combinations thereof.

In some embodiments is a synergist or co-agent or active co-agent for transforming a therapeutic potential of an antibiotic combination when utilized against at least one or more anaerobic bacteria, the antibiotic combination comprising: at least an effective amount or therapeutically effective amount of a diaminopyrimidine or an inhibitor of a microbial (bacterial) DHFR; and an effective amount or therapeutically effective amount of a sulfonamide or an inhibitor of a microbial (bacterial) DHPS, each when alone have some activity against an aerobic bacteria, the synergist comprising an effective amount or therapeutically effective amount of a fosfomycin or an inhibitor of a microbial (bacterial) MurA enzyme (comprising a stereoisomer, a geometric isomer, a tautomer, a hydrate, a solvate thereof, a pharmaceutically acceptable phosphate, salt, acid, amine, and/or ester thereof, and any combination thereof, and having some activity against an aerobic bacteria), such that the synergist or co-agent or active co-agent with the antibiotic combination are now therapeutically effective against the one or more anaerobic bacteria in an anaerobic environ or an infection caused by or suspected of being caused by the one or more anaerobic bacteria in an anaerobic environ, wherein the antibiotic combination alone, without the synergist or co-agent or active co-agent, is therapeutically less effective or therapeutically ineffective against the one or more anaerobic bacteria and/or the infection, wherein the antibiotic combination and the synergist or co-agent or active co-agent are in a same formulation or different formulations, wherein the antibiotic combination and the synergist or co-agent or active co-agent are provided at a same period or substantially same period or within about one hour or two hours or three hours, wherein the diaminopyrimidine or inhibitor of the microbial (bacterial) DHFR is a stereoisomer, geometric isomer, a tautomer, a hydrate, a solvate thereof, a pharmaceutically acceptable phosphate, salt, acid, and/or ester thereof, and combinations thereof, and wherein the sulfonamide or inhibitor of the microbial (bacterial) DHPS is a stereoisomer, geometric isomer, a tautomer, a hydrate, a solvate thereof, a pharmaceutically acceptable phosphate, salt, acid, and/or ester thereof, and combinations thereof.

In some embodiments is a synergist or co-agent or active co-agent for transforming a therapeutic potential of an antibiotic combination when utilized against at least one or more anaerobic bacteria in an anaerobic environment, the antibiotic combination comprising at least: (aa) an effective amount or therapeutically effective amount of a fosfomycin or an inhibitor of a microbial (bacterial) MurA enzyme (any of which comprise a stereoisomer thereof, a geometric isomer thereof, a tautomer thereof, a hydrate thereof, a solvate thereof, a pharmaceutically acceptable phosphate, salt, acid, amine, and/or ester thereof, and any combination thereof); (bb) an effective amount or therapeutically effective amount of a diaminopyrimidine or an inhibitor of a microbial (bacterial) DHFR (any of which comprise a stereoisomer, a geometric isomer, a tautomer, a hydrate, a solvate thereof, a pharmaceutically acceptable phosphate, salt, acid, and/or ester thereof, and any combination thereof); and (cc) an effective amount or therapeutically effective amount of a sulfonamide or an inhibitor of a microbial (bacterial) DHPS (any of which comprise a stereoisomer, a geometric isomer, a tautomer, a hydrate, a solvate thereof, a pharmaceutically acceptable phosphate, salt, acid, and/or ester thereof, and any combination thereof), each of (aa), (bb), (cc), when alone have some activity against an aerobic bacteria; the synergist or co-agent or active co-agent comprising an effective amount or therapeutically effective amount of any one or combination of: (dd) inhibitor of microbial (bacterial) peptidoglycan synthesis from a class of glycopeptide/lipoglycopeptide antibiotics that bind to an outer wall of a bacteria and inhibit or interfere with bacterial peptidoglycan synthesis and synthesis of its cell wall (comprising a stereoisomer, a geometric isomer, a tautomer, a hydrate, a solvate thereof, a pharmaceutically acceptable phosphate, salt, acid, and/or ester thereof, and combinations thereof); (ee) inhibitor of a microbial (bacterial) fosfomycin-modifying enzyme (comprising a stereoisomer, a geometric isomer, a tautomer, a hydrate, a solvate thereof, a pharmaceutically acceptable phosphate, salt, acid, and/or ester thereof, and any combination thereof); (ff) inhibitor of a microbial (bacterial) beta lactamase (comprising a stereoisomer, a geometric isomer, a tautomer, a hydrate, a solvate thereof, a pharmaceutically acceptable phosphate, salt, acid, and/or ester thereof, and combinations thereof), such that the synergist or co-agent or active co-agent with the antibiotic combination are therapeutically effective against the one or more anaerobic bacteria or an infection caused by or suspected of being caused by one or more anaerobic bacteria, wherein the antibiotic combination alone, without the synergist or co-agent or active co-agent, is therapeutically less effective or therapeutically ineffective against the one or more anaerobic bacteria and/or the infection, wherein the antibiotic combination and the synergist or co-agent or active co-agent are in a same formulation or different formulations, and wherein the antibiotic combination and the synergist or co-agent or active co-agent are provided at a same period or substantially same period or a period within about one hour or about two hours or about three hours.

Also herein is a plurality of co-agents or active co-agents for utilizing against at least one or more obligate anaerobes, or as a combination comprising a plurality of co-agents or active co-agents for utilizing against at least one or more obligate anaerobes, the co-agents comprising at least: an effective amount or therapeutically effective amount of a diaminopyrimidine or an inhibitor of a microbial (bacterial) DHFR in a pharmaceutically acceptable form (comprising a stereoisomer, a geometric isomer, a tautomer, a hydrate, a solvate thereof, a pharmaceutically acceptable phosphate, salt, acid, and/or ester thereof, and any combination thereof); an effective amount or therapeutically effective amount of a sulfonamide or an inhibitor of a microbial (bacterial) DHPS in a pharmaceutically acceptable form (comprising a stereoisomer, a geometric isomer, a tautomer, a hydrate, a solvate thereof, a pharmaceutically acceptable phosphate, salt, acid, and/or ester thereof, and any combination thereof); and an effective amount or therapeutically effective amount of a fosfomycin or an inhibitor of a microbial (bacterial) MurA enzyme in a pharmaceutically acceptable form (comprising a stereoisomer, a geometric isomer, a tautomer, a hydrate, a solvate thereof, a pharmaceutically acceptable phosphate, salt, acid, amine, and/or ester thereof, and any combination thereof), wherein each co-agent when used alone has some activity against an aerobic bacteria, wherein the plurality of co-agents are therapeutically effective and active against one or more obligate anaerobes in an anaerobic environment, the one or more obligate anaerobes causing or suspected of causing an infection in a host or subject, wherein the plurality of co-agents are provided to the host or subject by co-dosing in a period being within about one hour or about two hours or about three hours.

In some embodiments, the invention herein is a co-agent combination comprising a plurality of co-agents (or at least three active co-agents) in at least one pharmaceutically acceptable formulation for pharmacotherapy against one or more pathogenic anaerobes (e.g., Gram-positive and/or Gram-negative anaerobic bacteria), the plurality of co-agents being active and effective in an anaerobic environment, the co-agents being synergists, and reversing a resistance of at least one of the one or more pathogenic anaerobes to at least one of the co-agents in anaerobic environ (e.g., reversing resistance in one or more Gram-negative pathogens or obligate anaerobes that exhibited bacterial resistance to F singly, and/or reversing resistance in one or more Gram-negative and/or Gram-positive pathogens or obligate anaerobes that exhibited bacterial resistance to any one of D or S singly or D, S as a duo). In some forms, the one or more pathogenic anaerobes are in and/or on a host or subject, and the at least one pharmaceutically acceptable formulation is delivered or dispensed in one or more therapeutically acceptable conveyances or vehicles, and in a therapeutically effective amount, to the host or subject. Delivery or dispensing of the at least one pharmaceutically acceptable formulation causes or promotes an action some period of time thereafter, the action being at least one of: i) growth inhibitory activity against one or more pathogenic anaerobes; (ii) sustained growth inhibitory activity against one or more pathogenic anaerobes; (iii) elimination (and/or destruction) of one or more pathogenic anaerobes; (iv) sustained elimination (and/or destruction) of one or more pathogenic anaerobes; (v) preventative activity against formation of microbial colonies of one or more pathogenic anaerobes; (vi) sustained preventative activity against formation of microbial colonies of one or more pathogenic anaerobes; (vii) alleviation of an infection caused by or derived from or suspected of being caused by one or more pathogenic anaerobes (e.g., in a host or subject); (viii) control of an infection caused by or derived from or suspected of being caused by one or more pathogenic anaerobes (e.g., in a host or subject); (ix) prevention of an infection that would be caused by or derived from or suspected of developing from one or more pathogenic anaerobes (e.g., in a host or subject); (x) reduction in infection rate or alleviation of an infection caused by or derived from or suspected of being caused by one or more pathogenic anaerobes (e.g., in a host or subject).

In a method or combination herein, with an antibiotic combination, there may be a synergist or co-agent or a further synergist or further co-agent, being any one or any combination of: an inhibitor of a fosfomycin-modifying enzyme; an inhibitor of microbial (bacterial) peptidoglycan synthesis from a class of glycopeptide/lipoglycopeptide antibiotics that bind to an outer wall of a bacteria and inhibit or interfere with bacterial peptidoglycan synthesis and synthesis of the cell wall; an inhibitor of a microbial (bacterial) beta lactamase, an antimicrobial beta lactam antibiotic; and an antimicrobial, any of which may be a stereoisomer thereof, geometric isomer thereof, tautomer thereof, hydrate thereof, solvate thereof, pharmaceutically acceptable phosphate, salt, acid, and/or ester thereof, and combinations thereof, and each being in a pharmaceutically acceptable amount and/or effective amount. In any method or combination herein, a quinazoline herein (Q) may replace a diaminopyrimidine herein (D) to provide additional unpredictable co-agent combinations and/or formulations that unexpectedly cause synergistic action of co-agents and provide antimicrobial activity (as described herein) against one or more pathogenic anaerobes herein.

For a combination or antibiotic combination and/or method herein, a synergist or co-agent or a further synergist or further co-agent or combination or antibiotic combination will be provided in a pharmaceutically acceptable amount and/or an effective amount for synergistic action. For a combination or antibiotic combination and/or method herein, a synergist or co-agent or a further synergist or further co-agent or combination or antibiotic combination, will be provided in a pharmaceutically acceptable amount and/or an effective amount for sufficient and/or effective antimicrobial activity against a target anaerobe. For a combination or antibiotic combination and/or method herein, a synergist or co-agent or a further synergist or further co-agent or combination or antibiotic combination may cause or initiate or promote a response being one or more of: (a) reducing growth of one or more pathogenic anaerobes; (b) inhibiting growth of one or more pathogenic anaerobes; (c) suspending growth of one or more pathogenic anaerobes; (d) eliminating in whole or in part one or more pathogenic anaerobes; (e) reducing in whole or in part one or more pathogenic anaerobes; (f) interfering with in whole or in part one or more pathogenic anaerobes; and (g) altering or enhancing or promoting susceptibility of one or more pathogenic anaerobes through a synergy of active co-agents (e.g., active co-agents being synergists, having synergistic action and sufficiently interfering with the one or more pathogenic anaerobes in a manner that pathogenic anaerobes become susceptible to active co-agents when in a described combination and/or formulation), in which one or more pathogenic anaerobes may or may not be non-susceptible and/or intrinsically resistant to at least one co-agent when utilized independently and singly against a pathogenic anaerobe. For a method or combination and/or antimicrobial combination herein, an effective and/or sufficient antibacterial activity may be reflected in any one or more activities (i)-(x) herein.

In a combination or antibiotic combination and/or method herein, a plurality of co-agents or a synergist or a co-agent may comprise or may further comprise any one or more of: an inhibitor of a microbial (bacterial) fosfomycin-modifying enzyme (FMEI); an inhibitor of a microbial (bacterial) peptidoglycan synthesis (PGSI) (e.g., from a class of glycopeptide/lipoglycopeptide antibiotics that bind to an outer wall of a bacteria and inhibit or interfere with bacterial peptidoglycan synthesis and synthesis of its cell wall); an inhibitor of microbial (bacterial) beta lactamase (BLI); and a beta lactam antibiotic (e.g., a PGSI as a beta lactam antibiotic having antimicrobial activity), any of which may be in a pharmaceutically acceptable form, including a stereoisomer thereof, a geometric isomer thereof, a tautomer thereof, a hydrate thereof, a solvate thereof, a pharmaceutically acceptable phosphate, salt, acid, and/or ester thereof, and any combination thereof.

In a combination or antibiotic combination and/or method herein, the at least one type and/or species of anaerobic bacteria or pathogenic anaerobe is not susceptible or sensitive to at least one co-agent or synergist when used independently or singly against an anaerobic bacteria or pathogenic anaerobe, which may be assessed by a standardized in vitro AST for anaerobes. In a combination or antibiotic combination and/or method herein, the at least one type and/or species of anaerobic bacteria or pathogenic anaerobe is resistant to at least one co-agent or synergist when assessed independently or singly against an anaerobic bacteria or pathogenic anaerobe, which may be determined or identified by a standardized in vitro AST for anaerobes. In a method or combination and/or antimicrobial combination herein, a clinical sample or specimen may be used for evaluating susceptibility or resistance of at least one type and/or species of anaerobic bacteria or pathogenic anaerobe in response to the co-agent or the synergist or the combination or the antibiotic combination or the plurality of co-agents herein (via a standardized in vitro AST for anaerobes), in which a clinical sample may be obtained from a site of an infection or a site suspected of having or containing an infection, the site being any one or more of blood, urine, wound, sputum, rectum, fecal matter, throat, and/or from a surgical biopsy. In a combination or antibiotic combination and/or method herein, the one or more type and/or species of anaerobic bacteria or pathogenic anaerobes may be pathogenic obligate anaerobes. In a combination or antibiotic combination and/or method herein, the one or more type and/or species of anaerobic bacteria or pathogenic anaerobes are at least one of: a type and/or species of obligate anaerobe, a type and/or species of obligate anaerobe in a mixed population, a type and/or species of GPOA, a type and/or species of GNOA, a type and/or species of GPOA in a mixed population, a type and/or species of GNOA in a mixed population, a type and/or species of drug-resistant anaerobe (having intrinsic resistance to at least one co-agent or active co-agent or synergist when assessed independently and singly against the anaerobe, which may be identified by in vitro testing, and may be or may include an obligate anaerobe), a type and/or species of drug-resistant anaerobe (having acquired resistance to at least one co-agent or active co-agent when provided independently and singly against the anaerobe, assessed by in vitro testing, and may be or may include an obligate anaerobe), and any combination or arrangement thereof. In a combination or antibiotic combination and/or method herein, the one or more type and/or species of anaerobic bacteria or pathogenic anaerobes are in (e.g., living in) an anaerobic environment. In a combination or antibiotic combination and/or method herein, the at least one type and/or species of anaerobic bacteria or pathogenic anaerobe may be a GPOA in an anaerobic environment. In a combination or antibiotic combination and/or method herein, the at least one type and/or species of anaerobic bacteria or pathogenic anaerobe may be one or more of Actinomyces spp., Arcanobacterium spp., Atopobium spp., Bifidobacterium spp., Bilophila spp., Clostridioides spp., Clostridium spp., Collinsella spp., Eggerthella spp., Eubacterium spp., Finegoldia spp., Parvimonas spp., Peptococcus spp., Peptostreptococcus spp., Propionibacterium spp., and/or Cutibacterium spp., and/or a microaerophilic Streptococcus spp. (including one or more of S. anginosus, S. constellatus, S. intermedius, S. mutans, viridans streptococci), in an anaerobic environment. In a combination or antibiotic combination and/or method herein, the at least one type and/or species of anaerobic bacteria or pathogenic anaerobe may be one or more of Actinomyces spp., Arcanobacterium spp., Atopobium spp., Bifidobacterium spp., Bilophila spp., Collinsella spp., Eggerthella spp., Eubacterium spp., Finegoldia spp., Parvimonas spp., Peptococcus spp., Peptostreptococcus spp., Propionibacterium spp., and/or Cutibacterium spp., and/or a microaerophilic Streptococcus spp. (including one or more of S. anginosus, S. constellatus, S. intermedius, S. mutans), in an anaerobic environment. In a combination or antibiotic combination and/or method herein, the at least one type and/or species of anaerobic bacteria or pathogenic anaerobe may be a GNOA in an anaerobic environment. In a combination or antibiotic combination and/or method herein, the at least one type and/or species of anaerobic bacteria or pathogenic anaerobe may be any one or more of Aggretibacter spp., Bacteroides spp., Parabacteroides spp., Dethiosulfovibrio spp., Fusobacterium spp., Phocaeicola spp., Porphyromonas spp., Prevotella spp., Sutterella spp., Veillonella spp., in an anaerobic environment. In a combination or antibiotic combination and/or method herein, the at least one type and/or species of anaerobic bacteria or pathogenic anaerobe is an obligate pathogenic anaerobe (Gram-positive and/or Gram-negative) and is not any one or more of a Lactobacillus spp., Pediococcus spp., or Leuconostoc spp. In a combination or antibiotic combination and/or method herein, each of a Lactobacillus, Pediococcus, and Leuconostoc are insensitive to or not susceptible to the herein plurality of co-agents or combination or antibiotic combination with the synergist, when the co-agents and/or synergist are F, D, S or F, Q, S. In a combination or antibiotic combination and/or method herein, the anaerobic bacteria are in any one of a neutral pH, an acidic pH, and a basic pH, in which a pH is between about 5 and 8, or any range therebetween, and does not distract from synergistic action of co-agents.

In a combination or antibiotic combination and/or method herein, the synergist or co-agent or plurality of co-agents will enhance or augment or promote antibacterial activity in a synergistic manner and to an efficacious level, which may be evaluated in vitro in a clinical setting from a clinical sample obtained or taken from a host or subject having or suspected of having a pathogenic anaerobe in need of the one or more methods and/or combinations or antibiotic combinations described herein. In a combination or antibiotic combination and/or method herein, the combination or antibiotic combination or plurality of co-agents provide or promote a growth inhibitory effect against the at least one type and/or species of anaerobic bacteria or pathogenic anaerobes, which is greater than a growth inhibitory effect of one synergist or one co-agent or one active co-agent when assessed individually (singly) against a pathogenic anaerobe. In a combination or antibiotic combination and/or method herein, the synergist or co-agent or active co-agent may be in a pharmaceutically acceptable formulation and/or pharmaceutically effective formulation intended for treatment of an infection in or suspected of being in a host or subject. In a combination or antibiotic combination and/or method herein, the combination or antibiotic combination may be in a pharmaceutically acceptable form or formulation intended for treatment of an infection in or suspected of being in a host or subject in need thereof. In a combination or antibiotic combination and/or method herein, an infection from a pathogenic anaerobe or anaerobic bacteria (or suspected thereof) may be unresolved in absence of the synergist (without at least one synergist) or in absence of the plurality of co-agents. In a combination or antibiotic combination and/or method herein, the synergist and the antibiotic combination or the plurality of co-agents may be provided to a host or subject in need thereof in a substantially same manner and/or form. In a combination or antibiotic combination and/or method herein, the synergist and the antibiotic combination or the plurality of co-agents may be provided to a host or subject in need thereof in a different manner and/or form. In a combination or antibiotic combination and/or method herein, the synergist and/or the antibiotic combination and/or the plurality of co-agents are provided to a host or subject in need thereof by one or more routes (for delivery or dispensing or administration), which may be a same route or different routes, the one or more routes comprising one of retentate enema, suppository, enteral, intravenous, intraperitoneal, inhalation, intramuscular, subcutaneous, and oral (or a combination thereof).

In a combination or antibiotic combination and/or method herein, the antibiotic combination and the synergist or the plurality of co-agents may be provided to a subject or host in need thereof once (e.g., pre- or post-surgery, or preventatively), or on a schedule, the schedule being every two hours, and/or every three hours, and/or every four hours, and/or every six hours, and/or every eight hours, and/or every ten hours, and/or every twelve hours, or once daily, and may continue until infection is resolved in whole or in part or when appropriate. In a combination or antibiotic combination and/or method herein, the antibiotic combination and the synergist or the plurality of co-agents may be provided daily in a suitable and acceptable form for delivery, in which the fosfomycin or inhibitor of a microbial (bacterial) MurA enzyme is provided daily every four hours or every six hours or every eight hours at about 500 mg to about 4 g per dose; the diaminopyrimidine or inhibitor of a microbial (bacterial) DHFR is provided daily every four hours or every six hours or every eight hours at about 80 mg to about 640 mg; and the sulfonamide or inhibitor of a microbial (bacterial) DHPS is provided daily every four hours or every six hours or every eight hours at about 360 mg to about 3.6 g. In a combination or antibiotic combination and/or method herein, the antibiotic combination and the synergist are provided daily in a suitable and acceptable form for delivery, in which the fosfomycin or inhibitor of a microbial (bacterial) MurA enzyme is provided daily every four hours or every six hours or every eight hours at about 0.7 g to about 3.3 g; the diaminopyrimidine or inhibitor of a microbial (bacterial) DHFR is provided daily every four hours or every six hours or every eight hours at about 80 mg to about 160 mg; the sulfonamide or inhibitor of a microbial (bacterial) DHPS is provided daily every four hours or every six hours or every eight hours at about 360 mg to about 800 mg. In a combination or antibiotic combination and/or method herein, the antibiotic combination and the synergist or the plurality of co-agents are provided daily in a suitable and acceptable form for delivery, in which the fosfomycin or inhibitor of a microbial (bacterial) MurA enzyme is provided daily every four hours or every six hours or every eight hours at about 1.8 g to about 7.5 g; the diaminopyrimidine or inhibitor of a microbial (bacterial) DHFR is provided daily every four hours or every six hours or every eight hours at about 5 mg to about 20 mg; the sulfonamide or inhibitor of a microbial (bacterial) DHPS is provided daily every four hours or every six hours or every eight hours at about 25 mg to about 100 mg. In a combination or antibiotic combination and/or method herein, the antibiotic combination and the synergist or the plurality of co-agents are provided daily in a suitable and acceptable form for delivery, such as an oral form, in which the fosfomycin or inhibitor of a microbial (bacterial) MurA enzyme is provided twice daily at about 1.5 g to about 3 g per day; the diaminopyrimidine or inhibitor of a microbial (bacterial) DHFR is provided twice daily at about 160 mg to about 320 mg per day; the sulfonamide or inhibitor of a microbial (bacterial) DHPS is provided daily every four hours or every six hours or every eight hours at about 800 mg to about 1600 mg per day. In a combination or antibiotic combination and/or method herein, the antibiotic combination and/or the synergist or the plurality of co-agents are provided daily in a suitable and acceptable form for delivery, in which delivery of the antibiotic combination and/or the co-agent or synergist is at an effective concentration and is continuous, in order to promote consistent delivery of at least one co-agent at or above its MIC against the at least one type and/or species of anaerobic bacteria or pathogenic bacteria.

These and other embodiments are further described and/or contemplated below.

### DETAILED DESCRIPTION

Although making and using various embodiments are described below and in the accompanying tables, it should be appreciated that herein are provided many inventive concepts that may be embodied, in which exemplary embodiments serve as representative embodiments and without undue limitation to the invention. Embodiments discussed herein are novel and inventive, yet those skilled in the art will recognize that various substitutions and modifications may be made without departing from what is described herein and defined by the appended claims.

In pharmacotherapy, when an existing antimicrobial agent can be revived in a new and more useful combination, there is a great benefit to the community as a whole. This is especially true if such a revival unexpectedly identifies one or both of: enhanced therapeutic potential of an existing antimicrobial agent when provided in the new combination(s); and/or new uses as well as more effective uses of an existing antimicrobial agent when provided in the new combination(s) (as compared with a reduced effect or inaction of the existing agent when used alone, or when used in an alternate and less effective combination). As disclosed herein, in one or more embodiments, are provided new and more effective antibiotic combinations, some of which include at least one existing antimicrobial agent (being one that is currently approved for use as an antimicrobial), in which all combinations herein include a plurality of co-agents, at least one of which serves for synergistic enhancement of activity of the co-agents as therapy against anaerobic pathogens and/or infections, complications, diseases or disorders negatively influenced or impacted by the anaerobic pathogens.

Further disclosed herein are additional unexpected and unique benefits for combinations and/or formulations, which include new and not previously described modes of action, a broadened spectrum of activity (e.g., activity against resistant anaerobes including intrinsically resistant type or species and thereby intrinsically non-susceptible), more potency and improved or better antimicrobial efficacy. Any of said unexpected findings described herein extends the importance and utility of a herein combination and/or formulation, each comprising a plurality of co-agents, each co-agent being active due to an evaluated synergy, in which the plurality of co-agents include at least an inhibitor of microbial (bacterial) MurA, and an inhibitor of microbial (bacterial) DHFR, and an inhibitor of microbial (bacterial) DPTS. The co-agents herein generally include at least three co-agents or three or more co-agents, each co-agent from a different class of antibiotics. It is understood that co-agents herein (for herein combinations and/or formulations) may be extended to new members of their particular class of antibiotics (when having an antibacterial property at least against an aerobic bacteria).

In one or more embodiments, at least one of the plurality of co-agents may be a known antibiotic agent (currently approved for use as an antibiotic, active against an aerobic bacteria), being revived and/or repurposed for new or expanded utilization herein. More than one of the plurality of co-agents may be known, being revived and/or repurposed for new or expanded utilization herein. Each of the plurality of co-agents may be known, being revived and/or repurposed for new or expanded utilization herein. At least one of the plurality of co-agents may be a functional equivalent (e.g., functional analog thereof, function derivative thereof) to a known antibiotic, and in a same class as the known antibiotic (being active against an aerobic bacteria). More than one of the plurality of co-agents may be a functional equivalent to a known antibiotic, each being in a same class as its known antibiotic (each active against an aerobic bacteria), or each of the plurality of co-agents may be a functional equivalent to a known antibiotic, each being in a same class as its known antibiotic (each active against an aerobic bacteria). The plurality of co-agents may be three known antibiotics, each known and revived and/or repurposed for new or expanded utilization. The plurality of co-agents may be four known antibiotics, each known and revived and/or repurposed for new or expanded utilization. The plurality of co-agents may be five known antibiotics, each known and revived and/or repurposed for new or expanded utilization. The plurality of co-agents may be three active co-agents, one known and revived and/or repurposed for new or expanded utilization. The plurality of co-agents may be three active co-agents, two being known and revived and/or repurposed for new or expanded utilization. The plurality of co-agents may be four active co-agents, one being known and revived and/or repurposed for new or expanded utilization. The plurality of co-agents may be four active co-agents, two being known and revived and/or repurposed for new or expanded utilization. The plurality of co-agents may be four active co-agents, three being known and revived and/or repurposed for new or expanded utilization. The plurality of co-agents may be five active co-agents, one being known and revived and/or repurposed for new or expanded utilization. The plurality of co-agents may be five active co-agents, two being known and revived and/or repurposed for new or expanded utilization. The plurality of co-agents may be five active co-agents, three being known and revived and/or repurposed for new or expanded utilization. The plurality of co-agents may be five active co-agents, four being known and revived and/or repurposed for new or expanded utilization.

In some embodiments, a plurality of novel and unpredictable compositions and/or formulations (comprising a plurality of co-agents herein) have antibacterial activity and are effective against one or more pathogenic anaerobic bacteria (pathogenic anaerobe). A plurality of novel and unpredictable compositions and/or formulations have antibacterial activity and efficacy against one or more pathogenic anaerobes in an anaerobic environment. In some forms, a pathogenic anaerobe is also referred to as a target. In some forms, a pathogenic anaerobe is one or more type or species of a pathogenic obligate anaerobic bacteria. In some forms, a pathogenic anaerobe is one or more type or species of a Gram-positive anaerobic bacteria. In some forms, a pathogenic anaerobe is one or more type or species of a GPOA bacteria. In some forms, a pathogenic anaerobe is one or more type or species of a Gram-negative anaerobic bacteria. In some forms, a pathogenic anaerobe is one or more type or species of a GNOA bacteria. In some forms, a pathogenic anaerobe is in a mixed population, comprising a combination of at least: one or more type and/or species of a pathogenic anaerobe; and one or more type and/or species of a pathogenic aerobe. In one or more forms, a pathogenic anaerobe is or includes one or more type and/or species of a facultative anaerobe.

In one or more forms, the one or more type or species of the pathogenic anaerobe is or includes at least one type or species of anaerobe that cannot utilize oxygen and/or is inhibited by oxygen. In one or more forms, a pathogenic anaerobe exists in an oxygen-free environment and/or poorly oxygenated environment (e.g., where oxygen and/or free oxygen are limited or absent, such as when blood supply is impaired, which may be from a trauma, injury, obstruction, perforation, aspiration, and/or surgical manipulation). In one or more forms, the one or more type or species of the pathogenic anaerobe is or includes at least one type or species of pathogen that does not require oxygen for respiration. In one or more forms, the one or more type or species of pathogen is or includes at least one type or species of pathogen with an inability to aerobically metabolize. In one or more forms, the one or more type or species of pathogen is or includes at least one type or species of pathogen that cannot utilize oxygen, some being inhibited by oxygen and depends, instead, on other substances (other than oxygen) as well as alternative enzymatic pathways for growth, metabolism, and activity. In one or more forms, the one or more type or species of pathogen is or includes at least one type or species of pathogen that undergoes energy conversion and metabolism with alternative (different) enzymes and enzyme pathways as compared with aerobic microorganisms that utilize and convert oxygen for energy. In one or more forms, the one or more type or species of pathogen is or includes at least one type or species of pathogen with an ability to metabolize in the absence of oxygen, and/or to undergo sustained metabolism in the absence of oxygen.

A pathogenic obligate anaerobe is a strict anaerobe and cannot utilize oxygen, some being inhibited by oxygen. For obligate anaerobes, oxygen can be toxic and/or will affect key enzymes in said organism. An obligate anaerobe is compromised over time when in the presence of oxygen, and, hence, does not function properly, or fails to function and will then die. In one or more or in some forms, one or more type or species of pathogen is or includes at least one type or species of pathogen that is able to sustain growth in an environment that provides sources other than oxygen to obtain energy and/or is able to sustain growth through fermentation. In one or more embodiments, any combination of said one or more type or species of pathogens may be one or more type or species of pathogen as a target, residing in or supported by an anaerobic environment.

In one or more forms, an anaerobic environment includes an environment supporting pathogenic anaerobes described herein, and is an environment under low oxygen saturation or low oxygen tension. Atmospheric oxygen may be absent or substantially absent (e.g., oxygen-free or substantially oxygen-free) and able to support pathogenic anaerobes herein, and in particular, one or more obligate anaerobes, such as in in vitro testings, including testings herein. Similarly, atmospheric oxygen may be at or at about or up to about 0.5 percent (and able to support pathogenic anaerobes, such as, one or more obligate anaerobes), or atmospheric oxygen may be between about 0.5 percent oxygen and 2 percent oxygen (and able to support one or more obligate anaerobes), or oxygen may be at or at about or up to about 2 percent oxygen (and able to support one or more obligate anaerobes), or oxygen may be between about 2 and 8 percent oxygen (and able to support one or more obligate anaerobes). In some instances, atmospheric oxygen may be up to about 8 percent oxygen, and able to support one or more pathogenic anaerobes. It is understood that in blood, fully oxygenated blood is bound (not free) with 95-100% oxygen saturation, and below 90 or 91% saturation is considered poorly oxygenated. It is also understood that oxygenation of a tissue varies greatly depending on tissue type and/or location, with a median or average for “normal” oxygenated tissue, generally being greater than 20 mmHg or well above 20 mmHg, while a median or average for diseased tissue or cancer tissue, generally being below 20 mmHg or about or below 16 mmHg (excluding rectal disorders or cancer that may measure about 25 mmHg). For purposes herein, an anaerobic environment is generally considered to be a poor or low oxygen environment (as is understood in the field), or, in some cases, an oxygen-free environment or substantially oxygen-free. In some embodiments, an anaerobic environment supports the pathogenic anaerobe and does not generally promote sustained energy production utilizing oxygen. In addition, or as an alternative, in some forms, the anaerobic environment supports the pathogenic anaerobe, is not toxic to the pathogen, while having low oxygen tension or low oxygen saturation or no oxygen, or oxygen only in an amount that is not toxic to the pathogenic anaerobe. In addition, or as an alternative, in one or more forms, the anaerobic environment supports the pathogenic anaerobe, is not toxic to the anaerobe, and if oxygen in in the environment, it is less than an amount (e.g., as a percentage or saturation level) suitable for sustained aerobic metabolism. In addition, or as an alternative, an anaerobic environment supports a pathogenic obligate anaerobe, providing sources other than oxygen for the pathogen to obtain sustained energy. In addition, or as an alternative, in some forms, an anaerobic environment is a condition that predisposes the environment to an anaerobic infection in a host or subject. Certain conditions found in a host or subject having an anaerobic infection include: tissue necrosis; a foul-smelling discharge; an environment or existing infection leading to thrombophlebitis; no improvement (symptomatic and/or physical) after providing alternative antibiotics and/or no response to alternative antibiotics; presence of virulence factors associated with an anaerobic infection (e.g., adhesion factors (such as fimbriae, lectin), invasion factors (such as phospholipase C, lipopolysaccharides, proteases), factors involved in tissue destruction (such as fibrinolysis, acetylglucosaminidase, collagenase), and/or capsular resistance to phagocytosis); an immunocompromised host; and/or a suspected site of anaerobic activity (e.g., intestinal/colorectal lumen, site of local trauma, site of surgery, site of viscus or other perforation, site of tissue necrosis, site of impaired clearance of a sterile site (such as chronic sinusitis and/or pneumonia); and/or ulcer).

A pathogenic anaerobe is targeted by any combination and/or formulation described herein. In one or more forms, a pathogenic anaerobe targeted by a combination and/or formulation described herein is or includes one or more type or species of anaerobe in an anaerobic environment described above. In one or more forms, a pathogenic anaerobe targeted by a combination and/or formulation described herein is or includes one or more type or species of anaerobe that are oxygen intolerant. In one or more forms, a pathogenic anaerobe targeted by a combination and/or formulation described herein is or includes one or more type or species of anaerobe that do not utilize oxygen, and may survive and be pathogenic in the absence of oxygen. In one or more forms, a pathogenic anaerobe targeted by a combination and/or formulation described herein is or includes one or more type or species of anaerobe that do not utilize oxygen and are inhibited by oxygen. In one or more forms, a pathogenic anaerobe targeted by a combination and/or formulation described herein is or includes one or more type or species of an obligate anaerobe in an anaerobic environment.

In mammals, anaerobes exist, and reside in oxygen-free habitats or ones that are very poorly oxygenated as described above. Such habitats include, without limitation, mucus membranes of the mouth (oral cavity) and upper and lower respiratory tract, intra-abdominal cavity, lower gastrointestinal (GI) tract, and pelvic region (pelvic cavity), and regions such as gingival crevices, tonsillar crypts, tooth surfaces, nasal folds, hair follicles, colon, urethra, and vagina. When one or more anaerobes in a host are displaced from their usual habitat or are introduced elsewhere (e.g., when consumed or when there is a breakdown in the mucocutaneous barrier or when there is immunosuppression), anaerobe(s) can become pathogenic, resulting in a local or systemic infection and/or a site of infection. A site of infection is often anaerobic and acidic. Without limitation, a site of infection is may include one or more of the following: brain (from, e.g., abscess, subdural empyema, meningitis, from infection or perforation or surgery or shunt); blood (from, e.g., hematogenous seeding, sepsis, abscess, bacteremia, wound, from infection or perforation or surgery); oral cavity (from, e.g., abscess, cellulitis, otitis media, mastoiditis, from infection or perforation or surgery); dental region (e.g., from abscess, root canal infection, sinusitis, from infection or perforation or surgery or root canal); pulmonary cavity (e.g., in bronchi and/or blood, from pneumonia, hematogenous seeding, abscess, bronchiectasis, nosocomial and/or necrotizing pneumonia, pleural empyema, from aspiration or infection or perforation or surgery); skin and soft tissue (from, e.g., abscess, acne vulgaris, decubitus ulcer, impetigo, gangrene, devitalized tissue, dead or dying tissue, necrotizing cellulitis, necrotizing fasciitis, bite wound, diabetic foot, from infection or perforation or surgery); intraabdominal region (from, e.g., abscess, appendicitis, peritonitis, wound infection, from infection or perforation or surgery); pelvic region (from, e.g., female genital tract infection, urinary tract (UT) infection, colitis, from infection or perforation or surgery and/or malignancy of a pelvic organ or region); muscle (from, e.g., abscess, wound, gangrene, cancer, from infection or perforation or surgery, and/or in immunocompromised subject); and heart region (from, e.g., endocarditis, bacteremia, from infection or perforation or surgery and/or malignancy).

In one or more embodiments, unexpected combinations and/or formulations (comprising a plurality of co-agents) as contemplated and/or described herein will, via synergistic action, initiate or cause or effect or elicit, as a response, at least one of: (a) reduction in growth of one or more pathogenic anaerobes (or growth reduction); (b) inhibition of growth of one or more pathogenic anaerobes (or growth inhibition); (c) suspension of growth of the one or more pathogenic microorganisms (or growth suspension); (d) elimination in whole or in part of one or more pathogenic anaerobes (or microbial elimination); (e) reduction in whole or in part of one or more pathogenic anaerobes (or microbial reduction); (f) interference in whole or in part of one or more pathogenic anaerobes (or microbial interference); (g) alter and/or enhance and/or promote susceptibility of one or more pathogenic anaerobes (which may or may not include one or more previously non-susceptible pathogenic anaerobes and/or one or more intrinsically resistant pathogenic anaerobes). In one or more embodiments, responses (a)-(g) arise through a synergism and/or synergistic action of co-agents, in which one or more co-agents are active or become, and at least one co-agent is a synergist, and, in combination, synergistic co-agents initiate or affect or cause synergistic action, and provide sufficient and/or effective antimicrobial activity against the one or more pathogenic anaerobes (including those previously considered non-susceptible and/or intrinsically resistant to at least one co-agent in the combination and/or formulation utilized, when the at least one co-agent is provided independently (singly) to the pathogenic anaerobe). In one or more embodiments, susceptibility (to at least one of the one or more unexpected combinations and/or formulations) is identified by in vitro susceptibility testing of anaerobes, which is note-worthy, since, as reported by others of skill in the field, clinical outcome has been well correlated with in vitro susceptibility testing results, in which mortality rate has been found to be higher when an inactive therapy is provided (inactive therapy being identified as non-susceptibility of anaerobes to that therapy and/or misdiagnoses, as examples). In view of such reportings, improving clinical outcome may include providing one or more active combinations and/or formulations to a host or subject in need thereof, and undergoing one or more in vitro susceptibility testings of a clinical culture of a clinical specimen or sample obtained from the host or subject, the clinical specimen or sample having or suspected of having at least one anaerobic pathogen causing or is suspected of causing an infection in the host or subject. When in vitro activity of a clinical specimen is examined or analyzed, utilizing information therefrom may be useful a better therapeutic outcome, in which efficacious combinations and/or formulations described herein will serve as a meaningful representation of good clinical outcome.

Anaerobic pathogens in at least a clinical setting that are strict or obligate anaerobes are represented by, but are not limited to, the following: Actinomyces spp., Atopobium spp., Bacteroides spp. and Parabacteroides spp., Bifidobacterium spp., Bilophila spp., Clostridioides spp., Clostridium spp., Collinsella spp., Cutibacterium spp., Dethiosulfovibrio spp., Eggerthella spp., Eubacterium spp., Finegoldia spp., Fusobacterium spp., Parvimonas spp., Peptococcus spp., Peptostreptococcus spp., Phocaeicola spp., Porphyromonas spp., Propionibacterium spp. (some now Cutibacterium spp.), Prevotella spp., Sutterella spp., and Veillonella spp., and/or certain microaerophilic Streptococcus spp. (such as one or more of S. anginosus, S. constellatus, S. intermedius, S. mutans, viridans streptococci). Such anaerobes as pathogens are found, unexpectedly, to be responsive to one or more unexpected combinations and/or formulations contemplated and/or described herein, in which such a combination and/or formulation (comprising a plurality of co-agents) is active against anaerobes as described herein, when a combination and/or formulation described herein promotes or initiates or causes, as a response, at least one of or any combination of (a)-(g), as identified above.

Indeed, and unexpectedly, while the obligate anaerobes described herein are responsive to active combinations and/or formulations contemplated and/or described herein, most obligate anaerobes are generally unresponsive (or not susceptible) to current antibiotics indicated and approved for an aerobic bacteria or infection caused therefrom. It is not uncommon for such current antibiotics to be inactive against obligate anaerobes. And most current antibiotics are not active in an anaerobic environment. Importantly, most obligate anaerobes described herein are also unresponsive (or not susceptible) to at least one co-agent utilized in an active combination and/or formulation described herein (when that co-agent is used individually or singly against the obligate anaerobe in an anaerobic environment (as determined using an in vitro susceptibility test).

Gram-positive anaerobes pathogenic in at least a clinical setting are represented by, but are not limited to, one or more or any combination of obligate anaerobes: Actinomyces spp., Arcanobacterium spp., Atopobium spp., Bifidobacterium spp., Bilophila spp., Clostridioides spp., Clostridium spp., Collinsella spp., Eggerthella spp., Eubacterium spp., Finegoldia spp., Parvimonas spp., Peptococcus spp., Peptostreptococcus spp., Propionibacterium spp., and/or Cutibacterium spp.; and may also include certain microaerophilic Streptococcus spp. (e.g., one or more of S. anginosus, S. constellatus, S. intermedius, S. mutans, viridans streptococci). All GPOAs tested were responsive to an unexpected combination and/or formulation (comprising a plurality of co-agents), in which the plurality of co-agents promote or initiate or cause, as a response, at least one or any combination of (a)-(g) as identified above. Further data is provided in accompanying tables, showing that each combination and/or formulation tested herein was active against all pathogenic GPOAs tested, and each combination and/or formulation tested against a GPOA showed antibacterial synergy. It is evident that combinations and/or formulations (when comprising a plurality of co-agents as described herein) is an active antibacterial, in which co-agents are synergistic, having a synergistic action and sufficient and/or effective antibacterial activity against pathogenic GPOA. Neither the profound synergistic action nor the extent of activity that was found (utilizing various co-agent combinations and/or formulations against GPOA) could have been predicted. This is particularly relevant as GPOA tested was either not susceptible to or was intrinsically resistant to at least one co-agent utilized a tested combination (in which non-susceptibility or intrinsic resistance was identified when one co-agent (in an unexpected combination) was used independently and singly against a GPOA (shown in data or tables or described by others of skill in the field). Moreover, the level of activity and better potency found (against each GPOA tested in an anaerobic environment) will be extremely beneficial for co-agents designed to target anaerobic locations, especially because such locations may be compromised, having lower blood flow and less tissue oxygenation, neither of which affect efficacy of unexpected combinations and/or formulations described herein, since such combinations and/or formulations appear optimized (highly active and highly potent) even in an anaerobic environment.

GNOAs pathogenic in at least a clinical setting are represented by and include, but are not limited to, any one or more of or any combination of: Aggretibacter spp., Bacteroides spp., Parabacteroides spp., Dethiosulfovibrio spp., Fusobacterium spp., Phocaeicola spp., Porphyromonas spp., Prevotella spp., Sutterella spp., Veillonella spp. All GNOA tested were responsive to an unexpected combination and/or formulation (comprising a plurality of co-agents), in which the plurality of co-agents promote or initiate or cause, as a response, at least one or any combination of (a)-(g) as identified above. Further data is provided in the accompanying tables, indicating that each unexpected combination and/or formulation tested herein was active against the pathogenic GNOAs, and each combination and/or formulation tested against a GNOA showed antibacterial synergy. As such, it is evident that combinations and/or formulations (when comprising a plurality of co-agents as described herein) is an active antibacterial, in which co-agents are synergistic, having a synergistic action and sufficient and/or effective antibacterial activity against pathogenic GNOA. Neither the profound synergistic action nor the extent of activity that was found (utilizing various co-agent combinations and/or formulations against GNOAs) could have been predicted. Particularly relevant is that each GNOA tested was either not susceptible to or was intrinsically resistant to at least one co-agent from a tested combination (non-susceptible or intrinsically resistance when one co-agent (in an unexpected combination) was used independently and singly against a GNOA (shown in data or tables or described by others of skill in the field). Moreover, the level of activity and better potency found herein (against each GNOA in an anaerobic environment) will be extremely beneficial for co-agents designed to target anaerobic locations, especially because such locations may be compromised, having lower blood flow and less tissue oxygenation, neither of which affect efficacy of unexpected combinations and/or formulations described herein, since such combinations and/or formulations appear optimized (highly active and highly potent) even in an anaerobic environment.

It has been reported by others skilled in the field that Bacteroides (some now Phocaeicola), Prevotella, Porphyromonas, and Fusobacterium species may make-up one-third or more of clinical isolates from specimens obtained from an infection in a host (e.g., mammal or subject thereof having an infection), indicating the importance of findings herein that unexpected combinations and/or formulations herein will be therapeutically effective against most clinical obligate anaerobes, due to findings from the inventors and/or as provided herein of profound synergistic action of all tested combinations, all being shown to have effective antibacterial activity (as any one of more of responses (a)-(g) described above) against all obligate anaerobes tested. Utilizing an unexpected combination and/or formulation herein will, thereby, provide an effective antibacterial in a host or subject having or suspected of having an infection caused by one or more obligate anaerobes, in which such obligate anaerobes may likely reside in and/or be supported by an anaerobic environment. And, based on findings from the inventors and/or data herein, combinations are not only synergistic, all will have effective antibacterial activity (as any one of more of responses (a)-(g) as described above) against all obligate anaerobes in an anaerobic environment.

Interestingly and unexpectedly, there are certain anaerobes that are not responsive to any of the unexpected combinations and/or formulations tested (and none of the co-agent combinations and/or formulations were active or able to promote or initiate or cause a response in these non-responsive anaerobes). The non-responsive (non-susceptible) anaerobes include facultative species of Lactobacillus spp. (Gram-positive rod-shaped), Leuconostoc spp. (Gram-positive cocci), and Pediococcus spp. (Gram-positive cocci), each of which is a facultative anaerobe and considered by others of skill in the relevant field as bacteria beneficial and/or mutualistic to a mammalian host (residing in a normal or non-diseased microflora of a mammalian host, e.g., non-pathogenic). In view of such data, it is evident that unexpected combinations and/or formulations herein are not active against and, thereby, do not provide an antimicrobial function against bacteria beneficial and/or mutualistic to a mammalian host. Yet, unexpectedly and unpredictably, there are other facultative anaerobes, one that are generally considered pathogenic to a mammalian host (e.g., in a clinical setting) that are responsive (susceptible) to unexpected combinations and/or formulations described herein (in which a co-agent combination and/or formulation tested against a responsive facultative anaerobe causing or promoting or initiating, as a response, at least one of or any combination of (a)-(g), as identified above). The responsive facultative anaerobes include those identified by skilled artisans in the field as non-beneficial and are not considered mutualistic to a mammalian host. Anaerobes when pathogenic and/or when causing or suspected of causing an infection in a host and/or a subject are a suitable or responsive target for any of the unexpected and inventive compositions and/or formulations herein. A responsive target will include an obligate anaerobe susceptible to one or more co-agent combinations and/or formulations herein. A responsive target will include responsive facultative anaerobes (e.g., considered pathogenic in a clinical setting) that are susceptible to co-agent combinations and/or formulations herein.

The unexpected and unpredicted findings herein with regard to responsiveness and susceptibilities of only certain (responsive) facultative anaerobes (yet not all facultative anaerobes), while all obligate anaerobes are profoundly responsive (susceptible) to combinations and/or formulations herein in an anaerobic environment, provides further evidence of unpredictability as to the inventions herein disclosed. Moreover, while some pathogenic anaerobes (obligate or responsive facultative anaerobes) were susceptible to all co-agents of a herein described combination and/or formulation (when each co-agent was tested individually against the pathogen, utilizing an in vitro susceptibility test for anaerobes), there are some pathogenic anaerobes (obligate or responsive facultative anaerobes) that were not susceptible to one of the co-agents when tested individually, and there were other pathogenic anaerobes (obligate or responsive facultative anaerobes) that were not susceptible to two co-agents, when tested individually (utilizing an in vitro susceptibility test for anaerobes). Still other pathogenic anaerobes (obligate or responsive facultative anaerobes) were not susceptible to three co-agents, when tested individually (utilizing an in vitro susceptibility test for anaerobes). Without being bound by theory, it is contemplated that synergy of co-agents may correlate with an accumulation of by-products and/or from toxic metabolite build-up in an anaerobe, which is somehow heightened in obligate anaerobes, possibly due, at least in part, to a lack of certain enzymes in obligate anaerobes, and/or to having altered enzymatic pathways for energy production or metabolite conversion. However, this notion does not explain why there are differences in responsiveness (and differences in co-agent susceptibilities) for certain pathogenic facultative anaerobes, while there is a complete lack of responsiveness (non-susceptibility) with mutualistic or beneficial facultative anaerobes (e.g., in high levels in a normal or non-diseased microflora of a mammalian host). Higher, more effective synergy may be due, in part, to membrane characteristics that may differ in obligate anaerobes, particularly when a plurality of co-agents are combined.

Facultative anaerobes that are responsive (susceptible) to one or more unexpected combinations and/or formulations herein in an aerobic (atmospheric) environment and may be pathogenic in at least a clinical setting include: Gram-positive facultative anaerobes, such as Arcanobacterium (branching bacillus), Bacillus (rod-shaped, e.g., B. cereus, B. subtilis, B. anthracis), Enterococcus (cocci, e.g., E. faecalis, E. faecium), Staphylococcus aureus (cocci); and Gram-negative facultative anaerobes, such as Acinetobacter baumannii (coccobacillus), Burkholderia (rod-shaped, e.g., B. thailandensis, B. cepacia, B. mallei, B. pseudomallei), Enterobacter (rod-shaped), Escherichia (rod-shaped coliform, e.g., E. coli), Klebsiella (rod-shaped, e.g., K. pneumoniae), Pseudomonas aeruginosa (rod-shaped), Yersinia (rod-shaped, e.g., Y. pseudotuberculosis, Y. pestis). It is further noted that many facultative anaerobes tested were drug-resistant or multi-drug resistant to one or a plurality of drugs currently approved or indicated for use as an antibiotic against aerobic bacteria.

It has been reported by others skilled in the field that it is not unusual for an infection in a mammal or host (or a site of infection thereof) to include more than one type or species of anaerobes (e.g., via reported testings of clinical isolates from specimens obtained from an infection in a mammal). Cooperative activity between obligate anaerobic pathogens has been found with certain species of Clostridioides, Clostridium, Bacteroides, Bilophila, Fusobacterium, Phocaeicola and Prevotella, any of which are, to date, particularly virulent, and have unpredictable susceptibility patterns. In view of data and information herein, it is evident that utilizing one or more unexpected combinations and/or formulations herein, will or should, in one or more embodiments, inhibit cooperative activity between obligate anaerobes. And, utilizing one or more unexpected combinations and/or formulations herein will provide an inhibitory effect at a site of infection (e.g., inhibit growth, inhibit colony formation), by inhibiting cooperative activity between pathogenic obligate anaerobes in a mammal or host containing, having or suspected of having the infection.

It is also not unusual for an infection in a mammal or host (or a site of infection in a mammal or host) to have or include or comprise or be caused by one or more type or species of organisms, referred to herein as a mixed population. In a mixed anaerobic infection, there may be different anaerobically metabolizing pathogens. Such mixed populations may be successfully targeted with one or more combinations and/or formulations herein, being found effective as an antimicrobial against a wide spectrum of anaerobic pathogens. In other mixed infections, there may be different metabolizing pathogens, some anaerobic and some aerobic. These mixed populations may also be successfully targeted with one or more combinations and/or formulations herein, because the combinations and/or formulations have been found effective as an antimicrobial against a wide spectrum of anaerobic pathogens as well as aerobic pathogens, as previously identified by the inventors. In one or more embodiments, utilizing one or more unexpected combinations and/or formulations herein will promote or initiate or cause, as a response, at least one of or any combination of (a)-(g), as identified above, when a target is a mixed population, including a mixed population of anaerobes responsive in an anaerobic environment (e.g., obligate and/or responsive facultative anaerobes, supported by and/or sustained in an anaerobic environment).

In general, unexpected inventive compositions and/or formulations herein comprise a plurality of co-agents as active co-agents. The plurality may consist of or substantially consist of or may comprise three co-agents, as active agents for antimicrobial synergy (synergistic action). The plurality may consist of or substantially consist of or may comprise four co-agents, as active agents for antimicrobial synergy (synergistic action). The plurality may consist of or substantially consists of or may comprise five co-agents, as active agents for antimicrobial synergy (synergistic action). In some embodiments, unexpected compositions and/or formulations herein consists of or substantially consist of or comprise, for antimicrobial purposes, three co-agents, as active agents for antimicrobial synergy (synergistic action). In some embodiments, an unexpected compositions and/or formulation herein comprises, for antimicrobial purposes, three or more co-agents, as active agents for synergy (synergistic action). In some embodiments, unexpected compositions and/or formulation herein comprise at least three co-agents or three or more co-agents, as active agents for antimicrobial synergy (synergistic action), in which, in use, the at least three co-agents or three or more co-agents are provided against anaerobic pathogens in a pathogenic environ. In many embodiments, the pathogenic environ is an anaerobic environment as described elsewhere herein.

In inventor findings as well as data provided herein, three or more co-agents are combined, as a composition and/or formulation, and delivered to pathogenic anaerobes or a host or subject in need thereof, the host or subject having or suspected of having one or more pathogenic anaerobes or an infection caused by or suspected of being caused by such anaerobes. When the pathogenic anaerobe is an obligate anaerobe, all co-agent combinations have been antibacterial, exhibiting antibacterial activity and promoting or initiating or causing, as a response or effect, at least growth inhibition. In one or more embodiments, antibacterial activity, manifested or caused by an unpredictable combination and/or formulation herein against an anaerobic pathogen or an infection caused or suspected of being caused by the anaerobic pathogen, will include at least one of or any combination of the following outcomes or effects: (i) growth inhibitory activity against one or more pathogenic anaerobes; (ii) sustained growth inhibitory activity against one or more pathogenic anaerobes; (iii) elimination (and/or destruction) of one or more pathogenic anaerobes; (iv) sustained elimination (and/or destruction) of one or more pathogenic anaerobes; (v) preventative activity against formation of microbial colonies of one or more pathogenic anaerobes; (vi) sustained preventative activity against formation of microbial colonies of one or more pathogenic anaerobes; (vii) alleviation or improvement of an infection (or its symptoms) caused by or derived from or suspected of being caused by one or more pathogenic anaerobes (e.g., in a subject in need thereof); (viii) control of an infection (or its symptoms) caused by or derived from or suspected of being caused by one or more pathogenic anaerobes (e.g., in a subject in need thereof); (ix) prevention of an infection that would be caused by or derived from or suspected of developing from one or more pathogenic anaerobes (e.g., in a subject in need thereof); (x) reduction in infection rate of an infection caused by or derived from or suspected of being caused by one or more pathogenic anaerobes (e.g., in a subject in need). These are attributed to herein co-agent synergists, having synergism, and beneficial synergistic action when utilized in combinations and/or formulation. Inventors utilizing co-agent synergists herein also found, unexpectedly, unpredictably, and non-obviously, in some embodiments, that there is a reversal in susceptibility or activity of at least one co-agent, which when provided individually to an anaerobe described herein had been ineffective or inactive as an antibacterial.

The level of co-agent synergy found by inventors is profound, unexpected, and particularly unpredictable given that many responsive anaerobic pathogens tested were not susceptible but were resistant (intrinsically or acquired) to either one co-agent, or two co-agents, or three-co-agents when the co-agent was tested singly using a standardized in vitro method of testing. Unexpected and unpredictable because each co-agent provided is largely regarded by others of skill in the field as lacking a spectrum of activity against many anaerobes, and particularly lacking antimicrobial activity against strict or obligate anaerobes. There are actually no reported susceptibility breakpoints for any co-agent utilized herein against strict or obligate anaerobes. Yet, cumulative data from copious and exhaustive testings performed by the inventors have been obtained and support the unpredictable and unexpected findings herein, that a plurality of active co-agents herein display unexpected synergy (with synergistic action) and superior antimicrobial efficacy (as compared with a co-agent used singly, or as compared with a current antibiotic approved for antimicrobial use alone or as a duo) against a vast spectrum of anaerobes, including all strict or obligate anaerobe tested, and responsive facultative anaerobes tested, ones considered non-mutualistic or non-beneficial to a normal microflora in a mammalian host. Also, unexpectedly, synergy and synergistic action as well as antimicrobial activity of co-agent combinations tested against obligate anaerobes was much greater than against responsive facultative anaerobes, which will aid in overall effectiveness and efficacy of co-agent combinations and/or formulations herein, particularly when required in an anaerobic or poorly oxygenated environment, in which obligate anaerobes reside. Co-agent combinations and/or formulations herein will penetrate the blood-brain barrier, making them particularly beneficial, especially given that many currently approved antibiotics cannot cross a blood-brain barrier or do not perform or are poorly effective in an anaerobic environment. Sometimes, superior antibacterial activity and synergism of co-agents as identified herein may allow a concentration of each co-agent in a composition and/or formulation herein that would be necessary to inhibit growth to be at or below what would be required to correlate with and provide clinical cure (assessed, for example, by area under the concentration-time curve (AUC) and maximum concentration (Cmax) in view of clinical pharmacokinetics and/or pharmacodynamics). In some instances, lowering an effective and/or therapeutic dosage of at least one co-agent due to its synergism with co-agents may reduce toxicity of that one co-agent. Lowering an effective and/or therapeutic dosage of at least one co-agent due to its synergism with co-agents may, in one or more embodiments, reduce or remove a prior safety issues when/if there is a safety issue with at least one co-agent in the combination and/or formulation.

Data herein and in the accompanying tables clearly demonstrate a superior antimicrobial activity and synergistic action of co-agent combinations tested against an obligate anaerobic pathogen. With severe infections (e.g., in a hospital setting, caused by or suspected of being cause by one or more pathogenic anaerobes), susceptibility testing of a clinical specimen may still be performed, and may be recommended prior to introducing a herein described co-agent therapy, or prior to changing therapy and before providing the co-agent combination and/or formulation as therapy, or anytime during the co-agent therapy). Some examples of severe, or serious, or life-threatening infection that may be benefit from one or more susceptibility testing are brain abscess, bacteremia, or endocarditis, or infections that were initially treated with another agent under a failed empiric therapy and failed to respond to that unsuccessful empirical therapy, or infections that relapsed after initially being treated and initially responding to another agent under an unsuccessful empirical therapy that failed, or infections where an antibacterial will have a special role in outcome, or when a host in need thereof requires prolonged therapy (e.g., sepsis, septic arthritis, osteomyelitis, undrained abscess, infection of graft or prosthesis, toxic megacolon, as examples).

Because anaerobes are fastidious pathogens, they are difficult to grow anaerobically unless proper culture methods are used. Indeed, it has been recommended by others skilled in the field that isolate testings be performed on highly virulent pathogens for which susceptibility cannot be predicted, which include at least Bacteroides spp., Phocaeicola spp., Prevotella spp., Fusobacterium spp., Clostridioides spp., Clostridium spp., and Bilophila spp. Proper culture methods (agar dilution or broth microdilution) were used for data collection, including data provided herein. A standardized anaerobic AST was utilized for susceptibility testing, referred to as M11, Methods for Antimicrobial Susceptibility Testing of Anaerobic Bacteria, from CLSI, which allows for comparison of data and findings herein, or for when assessed prior to or during treatment in a clinical setting.

In brief, agar-dilution method was utilized for AST of strict anaerobes, in which Brucella agar media was used for plates (supplemented with 5% laked sheep blood, 5 μg/mL hemin, 1 μg/mL Vitamin K1, and 50 mg/L glucose-6-phosphate (G6P) according to CLSI recommendations). Co-agents were incorporated in a two-fold geometric dilution scheme. Incubation was anaerobic for ˜48 hours at 35 degrees C. (e.g., via Coy Anaerobe Chamber with 0 ppm oxygen, or BD GasPak EZ Anaerobe System). A checkerboard variant for each matrix was utilized to evaluate co-agent combinations. Plates were inoculated with anaerobic colonies (suspended in saline after removal from anaerobic cultures) using 0.5 MacFarland standard of turbidity from a pure culture incubated for 48 h and replica stamped). After plate incubation for 48-72 h in an anaerobic (oxygen-free) environment, plates were read (growth or no growth) and compared visually. The lowest concentration of agent(s) that inhibited growth of the given bacterial strain was the MIC. For facultative anaerobes, a similar agar dilution schema was utilized, in which the media was replaced with Mueller-Hinton agar supplemented with 50 mg/L G6P, and conditions were aerobic. All broth microdilution and agar-dilution methods are highly labor-intensive. The checkerboard matrices were used to determine synergy between co-agents, as understood in the field by one skilled in the art, and when ascertaining synergy, replicate series of checkerboard plates were prepared with addition co-agents included (a three-dimensional synergy arrangement). FICI were calculated using known methods. For three co-agents, FICI=FIC(A)+FIC(B)+FIC(C)=(A/MIC A)+(B/MIC B)+(C/MIC S), where MIC A and MIC B are MICs of a co-agent alone and A and B and C are MICs of each co-agent in combination, respectively. When two co-agents are already combined prior to testing (e.g., a duo such as a D and S duo, in certain examples), FICI=FIC(A)+FIC(B)=(A/MIC A)+(B/MIC B), where MIC A is MIC of a co-agent alone and MIC B is MIC of a co-agent duo and A and B MICs of each co-agent/co-agent duo at a fixed ratio in combination (for three co-agents), respectively. FIC indices define an antibacterial interaction as synergistic (S) when 115; additive (A) when >0.5-1; indifferent (I) when >1 but 4; or antagonistic (X) when >4.

Unexpectedly, unpredictably, and non-obviously, inventors have found that co-agents in combinations and/or formulations herein directed against certain responsive (e.g., pathogenic) facultative anaerobes described herein have longer PAE, as compared with PAE of one co-agent when used singly. In addition, or as an alternative, co-agent combinations and/or formulations herein have a same PAE at a lower concentration of at least one co-agent, as compared with PAE and concentration of one co-agent when used singly against the responsive facultative anaerobes.

Co-agents in one or more unexpected combinations and/or formulations are below.

1. Antibacterial fosfomycins or inhibitors of bacterial MurA (UDP-GlcNAc enolpyruvyl transferase). Fosfomycins (F) or inhibitors of bacterial MurA herein are provided in a suitable and pharmaceutically acceptable form, such as a salt, phosphate, acid, amine and/or ester. Currently, fosfomycins are the only inhibitors of MurA, an enzyme specific to bacteria. A fosfomycin or MurA inhibitor may be a phosphonate or phosphonic acid derivative; representative and non-limiting forms may include a salt, such as a disodium salt, or a monobasic hydrosoluble salt, a mono-salt, a di-salt (e.g., sodium, potassium, calcium, magnesium), a salt formed with amine(s) (e.g., α-phenethylamine, quinine, lysine, procaine, tromethamine), or chemically as (−)(cis-1,2-epoxy-propyl) phosphonic acid. Suitable fosfomycins or inhibitors of bacterial MurA will likely have some antibacterial activity (meaning active against at least one aerobic bacteria or at least one facultative anaerobe in an aerobic environment), behaving as a phosphoenolpyruvate (PEP) substitute, binding MurA, irreversibly inactivating and/or inhibiting the MurA enzyme in peptidoglycan biosynthesis and/or displaying inhibitory activity of bacterial cell wall synthesis, by inhibiting or inactivating its MurA enzyme and disrupting peptidoglycan synthesis. Any new and/or functionally equivalent analogs or derivatives being still in a same class as fosfomycins herein (as inhibitors of bacterial MurA, with or without one or more acceptable substitution(s)) may be substituted and utilized as a fosfomycin, as long as it acts functionally in a same manner as described above, including stereoisomers thereof, geometric isomers thereof, tautomers thereof, hydrates thereof, solvates thereof, and pharmaceutically acceptable phosphates, salts, acids, amines, and/or esters thereof, and any combination thereof. Representative examples are fosfomycin tromethamine, and phosphonic acid derivatives, such as but not limited to cis-1,2-epoxypropyl phosphonic acid, a monobasic hydrosoluble fosfomycin salt or fosfomycin-trometamol. Fosfomycins or inhibitors of bacterial MurA (or acceptable analogs or derivatives thereof) should penetrate a variety of mammalian tissue, and at least access fluid (blood, mucus, urine, plasma, cerebrospinal fluid), membranes (peritoneum, pericardium, mucous), and tissues of the central nervous system (CNS), soft tissue, bone, and lungs.

It is understood that fosfomycins as monotherapy are not recommended against pathogenic anaerobes, due, in part to an inherent microbial resistance to fosfomycins through MurA mutations and because fosfomycin haven't been found to deliver adequate activity against many anaerobes. Rapid development of resistance to fosfomycin in vitro has been reported by others of skill in the field, against several facultative anaerobes, including Pseudomonas aeruginosa, Acinetobacter baumannii, Escherichia coli, Klebsiella spp., and in some multi-drug resistant pathogens.

2. Antibacterial diaminopyrimidines as inhibitors of bacterial DHFR. Diaminopyrimidines (D) or inhibitors of bacterial DHFR herein are provided in a suitable and pharmaceutically acceptable form, such as a salt, phosphate, acid and/or an ester. Suitable diaminopyrimidines will likely have some antibacterial activity (meaning active against at least one aerobic bacteria or at least one facultative anaerobe in an aerobic environment), and will inhibit DHFR, disrupting folate synthesis in the pathogen. A suitable D or inhibitor of bacterial DHFR generally includes at least two amine groups on a pyrimidine ring (sometimes referred to as diaminopyrimidines). Any new and/or functionally equivalent diaminopyrimidine analogs or derivatives being still in a same class as a diaminopyrimidine herein (as inhibitors of bacterial DHFR and disrupting folate synthesis, with or without one or more acceptable substitution(s)) may be substituted and utilized as a diaminopyrimidine, as long as it acts functionally in a same manner as described above, including stereoisomers thereof, geometric isomers thereof, tautomers thereof, hydrates thereof, solvates thereof, and pharmaceutically acceptable phosphates, salts, acids, and/or esters thereof, and any combination thereof. Representative examples include at least: 2,4-diaminopyrimidine; 2,4-diamino-5-benzyl-pyrimidine; 2,4-diamino-5-(4-amino-3,5-dichlorobenzyl)-pyrimidine; 2,4-diamino-5-(3,5-dichloro-4-methylaminobenzyl)-pyrimidine; 2,4-diamino-5-(3,5-dichloro-4-ethylaminobenzyl)-pyrimidine; 2,4-diamino-5-(3,5-dichloro-4-dimethylaminobenzyl)-pyrimidine; 4-diamino-5-(4-acetamido-3,5-dichlorobenzyl)-pyrimidine; 2,4-diamino-5-(4-bromo-3,5-dimethoxybenzyl)-pyrimidine; 2,4-diamino-5-[3,5-diethoxy-4-pyrrol-1-yl)-benzyl]-pyrimidine, 2,4-diamino-5-(3,4-dimethoxybenzyl)-pyrimidine (diaveridine); 2,4-diamino-5-(p-chlorophenyl)-6-ethylpyrimidine (pyrimethamine); 2,4-diamino-5-(2-methyl-4,5-dimethoxybenzyl)-pyrimidine; (RS)-5-[(2-cyclopropyl-7,8-dimethoxy-2H-chromen-5-yl)methyl]pyrimidine-2,4-diamine (iclaprim), and 5-[(4-bromo-3,5-dimethoxyphenyl)methyl]pyrimidine-2,4-diamine (brodimoprim). A diaminopyrimidine may also include a diaminopyrimidine with substitutions in the phenyl ring, such as 2,4-diamino-5-(3,4,5-trimethoxybenzyl)-pyrimidine (trimethoprim), 2,4-diamino-5-[3,5-dimethoxy-4-(2-methoxyethoxy)benzyl]-pyrimidine (tetroxoprim), and 2,4-diamino-5-(3,5-dimethoxy-4-methylthiobenzyl)-pyrimidine (metioprim). Diaminopyrimidines as inhibitors of bacterial DHFR (or acceptable analogs or derivatives thereof) should penetrate a variety of mammalian tissue, and at least access fluid (blood, mucus, urine, plasma, cerebrospinal fluid), membranes (peritoneum, pericardium, mucous), and tissues of the CNS, soft tissue, bone, and lungs.

While certain diaminopyrimidines may be provided as monotherapy, a representative diaminopyrimidine, trimethoprim, is more often indicated with sulfamethoxazole. Herein, a diaminopyrimidine may be provided herein with a sulfonamide as a duo of co-agents. Resistance to trimethoprim alone has risen and is plasmid-mediated or from chromosomal gene or promoter mutations and/or highly mobile transposons. Development of resistance to trimethoprim in vitro and in vivo have been reported by others of skill in the field. Resistance in vitro is frequent in aerobes and facultative anaerobes, including Acinetobacter spp., Brucella spp., E. coli, Haemophilus influenzae, Enterobacteriales (e.g., E. coli), Neisseria spp., S. aureus, Chlamydia spp., Coxiella spp., Rickettsia spp., Mycobacterium tuberculosis, Mycoplasma, and Treponema, and in most pseudomonads (intrinsically resistant), and some multi-drug-resistant pathogens.

3. Antibacterial sulfonamides or inhibitors of bacterial DHPS. Sulfonamides (S) or inhibitors of bacterial DHPS are provided in a suitable and pharmaceutically acceptable form, such as a salt, phosphate, acid, and/or as an ester. A sulfonamide or inhibitor of bacterial DHPS is derived from sulfanilamide, and modified by one or more functional groups to the amino group or one or more substitutions on the amino group, some being in a form of an alkali metal salt. A suitable sulfonamide or inhibitor of bacterial DHPS will likely have some antibacterial activity (meaning active against at least one aerobic bacteria or at least one facultative anaerobe in an aerobic environment), and will inhibit DHPS, interfering with folic acid synthesis (or synthesis of folate). In one or more embodiments, a sulfonamide is represented by a structure depicted as structure I below, where R is an organic group.

RSO₂NH₂ I.

A sulfonamide may also be in a cyclic form (e.g., sultam), which may be a N¹-heterocyclic substituted sulfonamide, such as a 5- or 6-membered heterocycle (e.g., a pyrimidine, pyrazine, pyridazine, oxazole, isoxazole, thiazole or thiadiazole ring). The sulfonamide may also be a sulfinamide represented by a structure depicted as structure II below, as amides of sulfinic acid, in which R and R′ are some organic group, and may further comprise a chiral form (e.g., tert-butanesulfinamide, p-toluenesulfinamide, 2,4,6-trimethylbenzenesulfinamide).

R(S═O)NH)R II.

A sulfonamide may also be a disulfonimide represented by a structure depicted as structure III below, with two sulfonyl groups flanking an amine, in which R and R′ are organic groups.

R—S(═O)₂—N(H)—S(═O)₂—R′ III.

Any new and/or functionally equivalent analogs or derivatives being still in a same class as sulfonamides herein and an inhibitor of bacterial DHPS (with one or more acceptable substitution(s)) may be substituted and utilized as a sulfonamide, as long as it acts functionally in a same manner as described above, including stereoisomers thereof, geometric isomers thereof, tautomers thereof, hydrates thereof, solvates thereof, and/or pharmaceutically acceptable phosphates, salts, acids, and/or esters thereof, and any combination thereof. Representative and non-limiting examples include: sulfadiazine, sulfamethoxazole, sulfatroxazole, sulfamerazine, sulfadoxine, sulfadimethoxine, sulfamethazine, sulfapyrazole, sulfaquinoxaline, sulfachloropyridazine, sulfaguanidine, sulfalene, sulfametin, sulfamethoxine, sulfamethoxy-pyridazine, sulfamethylphenazole, sulfamethoxypyridazine, sulfaethoxypyridazine, sulfabromomethazine, sulfaphenazole, sulfamoxole, sulfapyrazine, sulfapyridazine, sulfapyridine, sulfasymazine, sulfathiozole, sulfametrole, sulfanilimide, sulfasomidine, and sulfisoxazole. Sulfonamides or inhibitors of bacterial DHPS (or acceptable analogs or derivatives thereof) should penetrate a variety of mammalian tissue, and at least access fluid (blood, mucus, urine, plasma, cerebrospinal fluid), membranes (peritoneum, pericardium, mucous), and tissues of the CNS, soft tissue, bone, and lungs.

While some sulfonamides may be provided as monotherapy, a sulfonamide, such as sulfamethoxazole, is often indicated with trimethoprim. Resistance to sulfonamides alone has risen greatly, limiting their use as a single agent and has restricted their clinical usefulness. Resistance is generally plasmid-mediated or chromosomal-mediated. Cross resistance is common. Acquired resistance in vitro is frequent as reported by others of skill in the field, found in many facultative anaerobes, including E. coli, Neisseria spp., Shigella spp., S. aureus, gonococci, meningococci, and pneumococci. Obligate anaerobes are not considered to be susceptible to sulfonamides.

4. Antibacterial quinazolines as inhibitors of bacterial DHFR. Quinazolines (Q) are alternative inhibitors of bacterial DHFR and are provided in a suitable and pharmaceutically acceptable form, such as a salt, phosphate, acid and/or an ester; acid forms include a hydrochloride or dihydrochloride. A quinazoline will likely have some antibacterial activity (meaning active against at least one aerobic bacteria or at least one facultative anaerobe in an aerobic environment), and will inhibit DHFR and/or bacterial thymidylate synthase in the pathogen. A suitable Q or inhibitor of bacterial DHFR is derived from naphthalene, with an aromatic heterocyclic structure that is bicyclic, having two fused six-membered rings (benzene ring and pyrimidine ring). A suitable quinazoline may be provided as a substitute for a diaminopyrimidine, providing the substituted quinazoline is an inhibitor of bacterial DHFR. Any new and/or functionally equivalent quinazoline analogs or derivatives being still in a same class as a quinazoline herein (as inhibitors of bacterial DHFR and disrupting folate synthesis, with or without one or more acceptable substitution(s)) may be substituted and utilized as an quinazoline, as long as it acts functionally in a same manner as described above, including stereoisomers, geometric isomers thereof, tautomers thereof, hydrates thereof, solvates thereof, and pharmaceutically acceptable phosphates, salts, acids, and/or esters thereof, and any combination thereof. Representative examples include: 3-benzyl-2-cinnamylthio-6-(methyl or nitro)-quinazolin-4(3H)-ones, derivatives of 4(3H)-quinazolinone or 4(3H)-quinazolin, 7-[(4-phenylphenyl)methyl]pyrrolo[3,2-f]quinazoline-1,3-diamine (irresistin-16), and N³-cyclopropyl-7-[([4-(1-methylethyl)phenyl]methyl]-7H-pyrrolo[3,2-f]quinazoline-1,3-diamine or as a dihydrochloride (SCH79797), anilinoquinazoline (gefitinib, lapatinib), aminoquinazoline (erlotinib, afatinib). A few quinazolines are under investigation, primarily for non-bacterial intervention. Resistance data is not available and activity against obligate anaerobes is not clinically known.

5. Inhibitors of bacterial FMEs. Inhibitors of bacterial FMEs (or FMEIs) are provided in a suitable and pharmaceutically acceptable form, such as a phosphate, salt, acid and/or an ester and prevent inhibition or inactivation of a fosfomycin herein. FMEs inactivate fosfomycins, and behave similar to metalloenzymes. FMEs include FosA (glutathione S-transferase or GST), FosB (bacillithiol S-transferase), FosC (GST), FosX (Mn²⁺-dependent epoxide hydrolase), kinase FomA, and kinase FomB, as well as and other related GSTs (FosA-type enzymes that are plasmid-borne, such as FosA3, FosA4, FosA5, and FosC2). Inhibitors of bacterial FMEs are generally small molecule inhibitors or chelators having a C—P bond (carbon to phosphorus, as phosphate or phosphonate group) and generally lacking amine groups, which include but not limited to phosphonoformate, phosphonoacetate, 2-phosphonobutyrate, 4-phosphonobutyrate, 2-phosphonoproprionate, 2-phosphonoproprionate, 3-phosphonoproprionate, methylphosphonate, ethylphosphonate, phenylphosphonate, acetylphosphonate, phosphonoacetaldehyde, sodium phosphonoformate, sodium phosphonoformate tribasic hexahydrate, triethyl phosphonoformate. Another example is a small molecule active site inhibitor of FosA: 3-bromo-6-[3-(3-bromo-2-oxo-1H-pyrazolo[1,5-a]pyrimidin-6-yl)-4-nitro-1H-pyrazol-5-yl]-1H-pyrazolo[1,5-a]pyrimidin-2-one. Any new and/or functionally equivalent analogs or derivatives that act as inhibitors of bacterial FMEs (with one or more acceptable substitution(s)) may be substituted and utilized as an inhibitor of FMEs, as long as it acts functionally in a same manner as described above, including stereoisomers thereof, geometric isomers thereof, tautomers thereof, hydrates thereof, solvates thereof, and pharmaceutically acceptable phosphates, salts, acids, and/or esters thereof, and any combination thereof.

6. Antibacterial inhibitors of bacterial peptidoglycan synthesis. Inhibitors of bacterial peptidoglycan synthesis (PGSIs) are herein represented by glycopeptide/lipoglycopeptide antibiotics and beta lactam antibiotics, and are provided in a suitable and pharmaceutically acceptable form, such as a salt, phosphate, acid, and/or as an ester. These inhibitors will either bind to lipid II precursor of bacterial outer wall (e.g., to peptidoglycan), inhibit peptidoglycan synthesis, and inhibit synthesis of the bacterial cell wall (glycopeptides/lipoglycopeptides), or bind to penicillin-binding protein enzyme that cross-link the cell wall and inhibit peptidoglycan synthesis (beta-lactams). Beta-lactam antibiotics have a beta-lactam ring (e.g., amoxicillin, ceftazidime, meropenem, as examples, and include four groups or classes: penicillins, cephalosporins, carbapenems, monobactams). Here, a PGSI may include penams (beta-lactamase sensitive (e.g., penicillin G, amoxicillin, piperacillin) or beta-lactamase resistant (e.g., methicillin, temocillin, oxacillin, cloxacillin)), cephems (more than 6 generations, exemplified below), carbapenems and penems (e.g., meropenem), and monobactams (e.g., aztreonam). New and/or functionally equivalent analogs or derivatives being still in this same class (as inhibitors of bacterial peptidoglycan synthesis, with one or more acceptable substitution(s)) may be substituted and utilized as a PGSI, as long as it acts functionally in a same manner as described above, including stereoisomers thereof, geometric isomers thereof, tautomers thereof, hydrates thereof, solvates thereof, and pharmaceutically acceptable phosphates, salts, acids, and/or esters thereof, and any combination thereof. Representative and non-limiting examples are cephems (e.g., ceftazidime, cefuroxime, cefoxitin, cefoperazone, ceftolozane, cefalexin, cefdinir, ceftriaxone, ceftaroline, cefixime, cefpodoxime, cefazolin, ceftibuten, ceftaroline, cefuroxime, cefotaxime, cefoperazone, cefepime, ceftolazane, ceftriaxone, ceftibiprole, cefiderocol, cefoxitin, moxalactam, S-64922, and the like, including 2^{nd} generation, 3^{rd} generation, 4^{th} generation, 5^{th} generation, and 6^{th} generation), vancomycin, teicoplanin, oritavancin, dalbavancin, telavancin, bleomycin, ramoplanin, decaplanin, diumycin A, coromyin, amoxicillin (other penicillins, e.g., ampicillin, carbenicillin, dicloxacillin, oxacillin, piperacillin, nafcillin, ticarcillin, as merely representative examples), any of which may be utilized for pharmacotherapy as a co-agent for purposes of the herein described inventions.

Many PGSIs are indicated or used in combination with an inhibitor of beta lactamase (BLI), and may be provided herein as a duo of co-agents or as independent co-agents. Amoxicillin is indicated or used with clavulanic acid (CA). Piperacillin is often indicated with tazobactam. Ceftolozane is often indicated with tazobactam. Ceftazidime may be indicated/used with avibactam. Ceftarolin may be indicated/used with avibactam. Cefpodoxime or ceftibuten or cefixime or other cephalosporins may be used with CA. Activity of these PGSIs are more effective against Gram-positive bacteria (primarily aerobes or facultative anaerobes). Resistance to PGSIs, largely as a class, is via expression of certain genes and/or transposons in the microorganism that can transfer to other Gram-positive bacteria. Resistance has been found against many facultative Gram-positive facultative anaerobes, including: enterococci, streptococci, staphylococci, and species of Listeria.

7. Inhibitors of bacterial beta-lactamase. Inhibitors of beta lactamase (BLIs) are a class of inhibitors and are provided in a suitable and pharmaceutically acceptable form, such as a salt, phosphate, acid, and/or as an ester. Inhibitors in the class act as a substrate that bind a bacterial beta-lactamase enzyme with high affinity or create or cause a sterically unfavorable interaction when binding to the bacterial beta-lactamase enzyme, and in binding or interfering with the enzyme, bacterial beta-lactamase is inhibited or inactivated. New and/or functionally equivalent analogs or derivatives being still in this same class (as inhibitors of bacterial beta lactamase, with one or more acceptable substitution(s)) may be substituted and utilized as a BLI, as long as it acts functionally in a same manner as described above, including stereoisomers thereof, geometric isomers thereof, tautomers thereof, hydrates thereof, solvates thereof, and pharmaceutically acceptable phosphates, salts, acids, and/or esters thereof, and any combination thereof. Representative and non-limiting examples are avibactam, relebactam, sulbactam, tazobactam, vaborbactam, CA, nacubactam, zidebactam, QPX7728, ETX0282 and VNRX5236.

A BLI without itself having beneficial antibacterial activity is utilized in combination with a PGSI (e.g., beta-lactam antibiotic or penicillin binding protein antibiotic), provided herein as a duo of co-agents or as independent co-agents. Avibactam may be used herein with ceftazidime or meropenem. CA may be utilized with amoxicillin, or with ticarcillin. Relebactam may be utilized with imipenem-cilastatin (indicated as a trio). Sulbactam may be utilized with ampicillin or with cefoperazone. Tazobactam may be utilized with piperacillin or with ceftolozane. Vaborbactam may be utilized with meropenem. Zidebactam may be utilized with cefepime. Nacubactam may be utilized with meropenem. Intrinsic and acquired resistance to several BLIs has been reported by others of skill in the field in certain facultative anaerobes, and continued emergence of resistance to BLIs is reported when they are combined with beta-lactam antibiotics. Intrinsic resistance against CA-amoxicillin occurs in at least the following: Citrobacter freundii, Enterobacter cloacae complex, Hafnia chief, Klebsielia aerogenes, Morganelia rnorganii, Plesiomonas shigelloides, Providencia rettgeri, Providencia stuartii, Serratia rnarcescens, Yersinia enterocolitica, Aeromonas hydrophlia, Aeromonas veronii, Aeromonas dhakensis, Aeromonas caviae, and Aerornonas jandoel. Sulbactam-ampicillin is not active against P. aeruginosa, Avibactam is not active against many metallo-beta lactamase (class B) producing bacterial strains, or A. baurnannii, Zidebactam does not appear to be active against A. baurnannii. Relebactam with imipenem-cilastatin is not active against bacteria with metallo-beta lactamases; the trio are poorly active against many Gram-negative bacteria that produce a beta lactamase known as oxacillinase or OXA-48. Nacubactam has a profile similar to avibactam.

With the above co-agent categories (1-7), a plurality of co-agents for use against one or more anaerobic pathogens may include, consist of, consist essentially of, or comprise three co-agents, in which co-agents are or include: F, D, and S; or F, Q, and S. The plurality of co-agents may include, consist of, consist essentially of, or comprise four co-agents, in which co-agents are or include: F, D, S, and FMEI; or F, Q, S, and FMEI; or F, D, S, and BLI (when BLI possesses its own antibacterial activity); or F, Q, S, and BLI (when BLI possesses its own antibacterial activity); or F, D, S, and PGSI; or F, Q, S, and PGSI. The plurality of co-agents may include, consist of, consist essentially of, or comprise five co-agents, in which co-agents are or include: F, D, S, FMEI, and PGSI; or F, Q, S, FMEI, and PGSI; or F, D, S, BLI, and PGSI; or F, Q, S, BLI and PGSI; or F, D, S, PGSI, and FMEI; or F, Q, S, PGSI, and FMEI. In one or more embodiments, at least one co-agent is a co-agent currently approved for use as an antibacterial, being revived and/or repurposed for utilization in the combination and/or formulation herein. Or, more than one co-agent may be a co-agent currently approved for use as an antibacterial, being revived and/or repurposed for utilization in the combination and/or formulation herein.

With any of such plurality of co-agents described above, co-agents are synergists, having synergism and synergistic action, providing or causing or promoting sufficient and/or effective antibacterial activity (as any one or more of (i) to (x) described herein) against pathogenic anaerobes in an anaerobic environment. Unexpectedly, such pathogenic anaerobes comprise at least one or more obligate anaerobes or one or more obligate anaerobes in a mixed population. Also, unexpectedly, while some facultative anaerobes are responsive to such plurality of co-agents, the responsiveness is selective, in which there are non-responsive facultative anaerobes selected from a group consisting of at least one of Lactobacillus, Leuconostoc, and Pediococcus (in which 100% of isolates of Lactobacillus, Leuconostoc, and Pediococcus were not responsive or not susceptible to any tested co-agent combination). Co-agent combinations and/or formulations comprising any of said plurality of co-agents, wherein co-agents are synergists, having synergism and synergistic action, providing or causing or promoting sufficient and/or effective antibacterial activity, are at least assessed by an in vitro AST for anaerobes, from which MIC and FIC values are determined. Also unexpectedly, co-agents having synergism and synergistic action, as identified and evaluated herein by MIC and FICI values, in which the plurality of co-agents is three co-agents, are less active than when the plurality of co-agents is four co-agents (or five co-agents), meaning there is more synergism with four co-agents (or five co-agents) than with three co-agents, keeping in mind that when a plurality of co-agents is three co-agents (e.g., F, D, and S; or F, Q, and S), the three co-agents remain and are highly effective as an antibacterial, acting synergistically, via synergistic action, and having profound antibacterial activity against pathogenic anaerobes described herein (e.g., active and having growth inhibitory activity against 100% of obligate anaerobe isolates tested, and against the responsive facultative anaerobe isolates tested), and not active against non-responsive facultative anaerobic isolates of Lactobacillus, Leuconostoc, or Pediococcus (at least when co-agents are F, D, S, or F, Q, S). And, when a plurality of co-agents is at least three co-agents (e.g., F, D, S; or F, Q, S), the at least three co-agents together being highly active and effective as an antibacterial, it is in spite of the fact that reports by others of ordinary skill in the relevant field have found that none of fosfomycin (as a representative F), or trimethoprim (as a representative D), or sulfamethoxazole (as a representative S), are individually considered an effective antibiotic against obligate anaerobes.

Given that there is strong correlation between bacterial susceptibilities assessed in vitro and clinical outcome of a subject or host having or suspected of having an infection caused by or suspected of being caused by the bacteria, findings herein provide evidence that a plurality of co-agents herein, in one or more pharmaceutically acceptable forms, may provide sufficient and/or effective antimicrobial activity against pathogenic anaerobes in a subject or host (having or suspected of having an infection caused by or suspected of being caused by the pathogenic anaerobe) when any of the plurality of co-agents herein are utilized as pharmaceutically acceptable pharmacotherapy against the pathogenic anaerobes in or suspected of being in the subject or host (or an infection presented or suspected in the subject or host). The pharmaceutically acceptable forms may be provided via a delivery or dispensing means described herein, and will deliver or dispense or distribute an effective amount of the plurality of co-agents to pathogenic anaerobes, such as in or suspected of being in a subject or host (or an infection presented or suspected the subject or host), for targeting pathogenic organisms, which are at least one or more type and/or species of obligate anaerobes, or one or more type and/or species of obligate anaerobes in a mixed population, without targeting non-responsive facultative anaerobic isolates of Lactobacillus, Leuconostoc, and/or Pediococcus. The pharmaceutically acceptable forms combined should be especially effective against pathogenic anaerobes responsible for, causing or suspected of causing a serious infection (e.g., requiring hospitalization and/or parenteral antibacterial therapy of a host or subject), or a complicated infection (e.g., in a host or subject, having an underlying and/or predisposing condition and/or unresponsive or resistant to a prior, alternative therapy). The delivery or dispensing means that delivers or dispenses or distributes an effective amount of the plurality of co-agents to pathogenic anaerobes provides co-agents in a same formulation or conveyance, or in one or more different formulations or conveyances. The delivery or dispensing means that delivers or dispenses or distributes an effective amount of the plurality of co-agents to pathogenic anaerobes provides co-agents in any of: each co-agent simultaneously; at least two co-agents simultaneously (and providing any other co-agent by any means herein in a period); at least three co-agents simultaneously (and providing any other co-agent by any means herein in a period); each co-agent together; at least two co-agents together (and providing any other co-agent by any means herein in a period); at least three co-agents together (and providing any other co-agent by any means herein in a period); each co-agent in series, each co-agent within a same or overlapping cycle or schedule; each co-agent in a period, the period being at about or less than about 15 min, or 30 min, or 45 min, or 1 hour (hr), or 2 hrs, or 3 hrs, or 4 hrs, or 5 hrs.

In one or more embodiments, more than a sufficient amount or sufficient concentration of F in any of a plurality of co-agents provided in a period, will improve efficacy of the combination and/or formulation (e.g., improved or better than F when is used singly against one or more obligate anaerobes, one or more GPOAs and/or one or more GNOAs; improved or better than when F in a co-agent combination and/or formulation is provided in a sufficient amount or sufficient concentration). In one or more embodiments, more than a sufficient amount or sufficient concentration of D in any of a plurality of co-agents provided in a period, will improve efficacy of the combination and/or formulation (e.g., improved or better than D when is used singly against one or more obligate anaerobes, one or more GPOAs and/or one or more GNOAs; improved or better than when D in a co-agent combination and/or formulation is provided in a sufficient amount or sufficient concentration).

The accompanying tables provide evidence of synergistic action of combinations and/or formulations herein, any of which will comprise three or more active co-agents as described (e.g., at least F, D, S or at least F, Q, S), in which MIC assays described are central to susceptibility evaluation against pathogens, as obligate anaerobic bacteria in an anaerobic environment (Gram-positive or Gram-negative, any of which grow in an absence of oxygen, growing anaerobically, and are not facultative anaerobes); and facultative anaerobic bacteria in an aerobic environment (Gram-positive or Gram-negative, any of which grow in oxygen, growing aerobically, and are not obligate anaerobes). It is noted that bacteria that are susceptible or resistant to one or more co-agents were included in the analyses. For convenience, a diaminopyrimidine and a sulfonamide were prepared as independent stocks, and provided together, as a duo, unless noted otherwise. Generally, such a duo was at a fixed ratio (e.g., 1:19 for D/S, which is a usual ratio in the clinical settings). Other known duos were provided together, often prepared as a duo when available as such. Known techniques were used to provide and maintain an oxygen-free (anaerobic) environment, with either GasPak EZ anaerobe system (in 7-liter anaerobe boxes) or a Coy Anaerobe chamber metered with 90% N₂-10% H₂ containing 0% oxygen.

Referring to TABLE 1, antibacterial synergy was achieved with a combination of three co-agents (a fosfomycin (F), here being fosfomycin sodium); a diaminopyrimidine (D), here being trimethoprim); and a sulfonamide (S), here being sulfamethoxazole) when tested against many different GPOA bacteria and many different GNOA bacteria in an anaerobic environment. Five different strains of C. perfringens were tested, three different strains of C. difficile were tested, and numerous strains of Bacteroides spp. or Phocaeicola spp. were tested. Co-agents were remarkably effective against all representative obligate anaerobic pathogens, reducing MIC values significantly. Complete co-agent synergy was shown against every strain tested, in which MIC for each co-agent combination was reduced to a level understood by others of skill in the art as synergistic. TABLE 1 showed that in a true anaerobic environment, a three co-agent combination (F, D, S) has exceptional antibacterial activity, exhibiting synergism (as synergists), being effective against all GNOAs, including but not limited to at least Bacteroides, Parabacteroides, Phocaeicola, Prevotella, Porphyromonas, and Aggretibacter. Nearly all GNOAs in TABLE 1 were non-responsive and/or not susceptible to F when it was provided singly to that GNOA, meaning F did not have antibacterial activity singly against most GNOAs, and would be ineffective (not therapeutically useful as single treatment) against an infection having or suspected of having a GNOA, such as Bacteroides, Phocaeicola, Parabacteroides, Prevotella, and/or Porphyromonas. Similarly, GNOAs were generally non-responsive and/or not susceptible to one or both of D and S, when provided without F. This means, in an anaerobic setting, one or both of D and S, when provided without F, do not have antibacterial activity against most GNOAs, and therefore, when provided singly or as a duo, will be ineffective (and not therapeutically useful as treatment) for an infection having or suspected of having a GNOA, such as Bacteroides, Parabacteroides, Phocaeicola, Prevotella, and/or Porphyromonas. In the tables, susceptibility is based on extrapolated MIC breakpoints for susceptible facultative anaerobic bacteria or aerobic bacteria in an aerobic (atmospheric oxygen) environment, which is ≤32 μg/ml for F (e.g., fosfomycin sodium) and ≤2 μg/ml for D (e.g., trimethoprim) and ≤38 μg/ml for S (e.g., sulfamethoxazole), and resistance to F (e.g., fosfomycin) being >64 μg/ml, resistance to D (e.g., trimethoprim) being >2 μg/ml (or 4 μg/μl or greater) and resistance to S (e.g., sulfamethoxazole) being >38 μg/ml (or 76 μg/ml or greater).

TABLE 1 also showed that in an anaerobic environment, a three co-agent combination (F, D, & S) has exceptional antibacterial activity, exhibiting synergism (as synergists), being effective against all GPOAs, including but not limited to at least Clostridioides, Clostridium, Bifidobacterium, Peptostreptococcus, Propionibacterium, and/or Cutibacterium, and a microaerophilic Streptococcus (e.g., Streptococcus mutans). Several GPOAs in TABLE 1 were non-responsive and/or not susceptible to F when it was provided singly to that GPOA, meaning F does not have antibacterial activity singly against these GPOAs, and would thereby be ineffective (not therapeutically useful as single treatment) against them (or in a host having or suspected of having them). And, GPOAs were generally non-responsive and/or not susceptible to one or both of D and S, when provided without F. This means, in a true anaerobic environment, one or both of D and S, when provided without F, do not have antibacterial activity against most GPOAs, and therefore, one or both of D and S provided singly or as a duo, will be ineffective (and not therapeutically useful as treatment) against an infection having or suspected of having many GPOAs, such as Clostridioides, Clostridium, Peptostreptococcus, Propionibacterium, and/or Cutibacterium, and/or a microaerophilic Streptococcus (e.g., Streptococcus mutans). Together, this means each obligate anaerobe was non-responsive to at least one of F or D or S, when tested singly or as a duo, which, on its own, is sufficient to remove any reason for expecting synergy with F and D and S. In addition, co-agent ineffectiveness against many obligate anaerobes, when delivered singly or as a duo, means there is no reason or justification for expecting the high level or high degree of antibacterial activity with co-agents comprising at least F, D and S.

TABLES 2, 3, 5, 6, 8 and 9 emphasize that a sulfonamide is a sufficient and effective co-agent as long as it has some antibacterial activity (against an aerobic bacteria) and is an inhibitor of bacterial DHPS, and, as such, will provide exceptional antibacterial activity as a co-agent with F and D, in which such co-agents exhibit synergism (as synergists), because, in every instance, FIC values showed complete synergy against every strain tested (in which MICs of co-agent combinations in said tables were all significantly reduced, and FIC indices were all identified herein as synergistic). TABLES 2, 3, 5, 6, 8 and 9, like TABLE 1, show that co-agents (being at least F and D and S) are synergistic, and have sufficient and/or effective antibacterial activity against all Gram-negative and GPOAs tested, and, therefore, will be effective (and therapeutically useful as treatment) against an infection having or suspected of having a GPOAs (such as at least Clostridioides, Clostridium, Bifidobacterium, Peptostreptococcus, Propionibacterium, and/or Cutibacterium, and/or a microaerophilic S. mutans) and/or a GNOA (such as at least Bacteroides, Phocaeicola, Parabacteroides, Prevotella, Porphyromonas, and/or Aggretibacter). In TABLES 2, 5, and 8, S is sulfisoxazole. In TABLES 3, 6, and 9, S is sulfadoxine. TABLES 2, 3, 5, 6, 8 and 9 show that replacement of one sulfonamide with another will still initiate or cause or promote a synergistic effect with F and D co-agents, such that at least three co-agents as synergists is preserved as well as their effectiveness as an antibacterial, having activity against one or more obligate anaerobes in an anaerobic environment.

TABLES 4-9 provide evidence that a diaminopyrimidine is a sufficient and effective co-agent as long as it has some antibacterial activity (against aerobic bacteria) and is an inhibitor of bacterial DHFR, and, as such, will provide exceptional antibacterial activity as a co-agent with F and S, in which co-agents exhibit synergism (as synergists), because, in every instance, FICIs showed complete synergy against every strain tested (MICs of all co-agent combinations were significantly reduced, and FICIs identified herein were all synergistic). TABLES 4-9, like TABLE 1, each show that co-agents (being at least F, D, S) are synergistic, and have sufficient and/or effective antibacterial activity against all GNOAs and GPOAs tested, and, therefore, will be effective (and therapeutically useful as treatment) against an infection having or suspected of having a GPOs (such as at least Clostridioides, Clostridium, Bifidobacterium, Peptostreptococcus, Propionibacterium, and/or Cutibacterium, and/or a microaerophilic S. mutans) and/or a GNOA (such as at least Bacteroides, Parabacteroides, Phocaeicola, Prevotella, Porphyromonas, and/or Aggretibacter). In TABLES 4-6, D is pyrimethamine. In TABLES 7-9, D is iclaprim. Together, TABLES 4-9 show that replacement of one diaminopyrimidine with another with another will still initiate or cause or promote a synergistic effect with F and D co-agents, such that at least three co-agents as synergists is preserved as well as their effectiveness as an antibacterial, having activity against one or more obligate anaerobes in an anaerobic environment.

In TABLES 1-9, co-agent synergists promote and potentiate antibacterial activity (e.g., at least as inhibition of bacterial growth and/or inhibition of colony formation and/or bacterial reduction) in an anaerobic environment, even when GNOAs (all but one) exhibit resistance to F, and nearly all GPOAs and nearly all GNOAs exhibited resistance to one or both of D and S. TABLES 1-9 provide evidence that one or both of D and S are generally ineffective against both GPOAs and GNOAs in an anaerobic environment. F is also shown to be ineffective against GNOAs in an anaerobic environment, which is consistent with reports by others of ordinary skill in the field that F, or D and/or S, lack sufficient activity when given in a safe concentration, singly or as a duo, against more difficult or pathogenic obligate anaerobes. Yet, despite individual resistance profiles and lack of activity of co-agents (F when singly delivered or D, S as a duo), it is clear from TABLES 1-9 that synergy always occurs when co-agents F & D & S are delivered in an anaerobic environment to any obligate anaerobic pathogen. Importantly, synergy, at least in part, appears responsible for reversing resistance in one or more GNOAs that exhibited bacterial resistance to F singly, and/or reversing resistance in one or more GNOAs and/or GPOAs that exhibited bacterial resistance to any one of D or S singly or D, S as a duo. As such, in view of TABLES 1-9, it is clear that as long as there is an F co-agent, there are numerous different sulfonamides as well as numerous different diaminopyrimidines that may serve as suitable co-agents, while achieving a same or similar (and sufficient) outcome of effecting meaningful antibacterial activity against obligate anaerobic pathogens in an anaerobic environment.

Referring to TABLE 10A, combined data shows that co-agents, in which there is a substitution of a diaminopyrimidine with an quinazoline (Q, represented by SCH79797), so that co-agents are at least F, Q, and S, are consistent with findings from TABLES 1-9. In TABLE 10A, co-agents, being at least F, Q, and S, are synergistic because, in every instance, complete synergy was identified against every strain tested (in which MICs were all significantly reduced and FIC indices identified herein and by others of skill in the art were all synergistic). This means co-agents of F, Q, and S, having sufficient and effective antibacterial activity against all GNOAs and GPOAs tested, will be effective (and therapeutically useful as treatment) against an infection having or suspected of having a GPOA (such as at least Clostridioides, Clostridium, Bifidobacterium, Peptostreptococcus, Propionibacterium, and/or Cutibacterium, and/or a microaerophilic S. mutans) and/or a GNOA (such as at least Bacteroides, Parabacteroides, Phocaeicola and/or Prevotella, as well as or alternatively, Porphyromonas and/or Aggretibacter). Without being bound by theory, Q is a suitable substitute for D when Q has some activity against aerobic bacteria and inhibits bacterial DHFR. While this inhibitory activity against DHFR may not be a sole explanation as to synergism between D, S, and F, it is suggested that the inhibitory effect on bacterial DHFR is, at least in part, how a substitution with Q provides a similar and effective synergism. Taking this into account and extrapolating with a susceptibility breakpoint based on D (trimethoprim), TABLE 10A also shows that Q when delivered singly is ineffective against GPOAs tested and against many GNOAs tested. Yet, despite the individual lack of activity of Q when singly delivered, it is clear from TABLE 10A that synergy always occurs when co-agents F & Q & S are delivered in an anaerobic environment to any obligate anaerobic pathogen. The MIC values for co-agents F & Q & S, show the co-agents are an effective antibacterial combination against GPOAs and GNOAs in an anaerobic environment. As was found with TABLES 1-9, it is clear from TABLE 10A that as long as there is a F co-agent, there are at least numerous different sulfonamides as well as numerous different quinazolines that may serve as suitable co-agents having synergism, while also achieving a same or similar and/or sufficient outcome of effecting meaningful antibacterial activity against obligate anaerobic pathogens in an anaerobic environment.

TABLE 10B confirms co-agent synergism, and causing of antibacterial activity of F, Q, and S co-agents against Gram-positive and Gram-negative facultative anaerobes in an aerobic environment (also noted in an anaerobic environment, data not shown). TABLE 10C confirms that synergy of a duo Q with S, is consistent with synergy of a duo D with S, when each duo is delivered in an aerobic environment against different strains of methicillin-resistant Gram-positive facultative anaerobes, and a plurality of drug-resistant Gram-negative anaerobes. TABLES 10A-10C suggest that when other discovered inhibitors of bacterial DHFR are identified (e.g., inhibiting bacterial DHFR, and having at least some activity against aerobic bacteria in an aerobic condition), other discovered inhibitors can be suitable for synergistic action with at least F and S, when replacing D (or Q).

TABLE 11 shows unexpected synergistic action caused by a combination of more than three co-agents, in which co-agents are F, D, S, a PGSI and a BLI, five co-agents that in general exhibit better synergism, and synergistic action, as compared with F, D, S or F, Q, S. In TABLE 11, co-agent synergists promote and potentiate antibacterial activity (e.g., as at least inhibition of growth and/or inhibition of colony formation) in an anaerobic environment, the more than three co-agents being more effective as an antibacterial against both GPOAs (e.g., at least Clostridioides, Clostridium, Bifidobacterium, Peptostreptococcus, Propionibacterium, and/or Cutibacterium) and GNOAs (e.g., at least Bacteroides, Parabacteroides, Phocaeicola and/or Prevotella) when co-agents further comprise a PGSI (here amoxicillin) and a BLI (herein CA) with F, D, and S. In TABLE 11, with two further co-agents, MIC values against all tested obligate anaerobes are dramatically reduced, due at least in part to five co-agents being better synergists, displaying better synergistic action in an in vitro anaerobic environment as compared with three co-agents. Having more than three co-agents likely provides a broader spectrum of activity than F, D, S, or F, Q, S.

Like TABLE 11, TABLE 12 shows unexpected synergistic action caused by a combination of more than three co-agents, in which co-agents are F, D, S, and a PGSI, four co-agents that in general exhibit better synergism, and synergistic action, as compared with F, D, S or F, Q, S. In TABLE 12, co-agent synergists promote and potentiate antibacterial activity (e.g., as at least inhibition of growth and/or inhibition of colony formation) in an anaerobic environment, the more than three co-agents being more effective as an antibacterial against both GPOAs (e.g., at least Clostridioides, Clostridium, Bifidobacterium, Peptostreptococcus, Propionibacterium and/or Cutibacterium) and GNOAs (e.g., at least Bacteroides, Parabacteroides, Phocaeicola and/or Prevotella) when co-agents further comprise a PGSI (here vancomycin) with F, D, and S. In TABLE 11, with a further co-agent, MIC values are reduced, due at least in part to four co-agents being better synergists, displaying better synergistic action in an in vitro anaerobic environment as compared with three co-agents. Again, having more than three co-agents also likely provides a broader spectrum of activity than F, D, S, or F, Q, S.

Bacterial strains in TABLES 1-9, 10A, 11, and 12, are representative of obligate anaerobic bacterial types and/or species that cause about or greater than about 95% of more difficult to treat anaerobic bacterial infections in humans. It is further noted that, as reported by others of ordinary skill in the field, in vitro MIC susceptibility values are known to correlate with good or efficacious clinical outcomes, meaning that tables and data herein showing susceptibility of all obligate anaerobes to co-agents combinations herein are not only useful, but serve as valid predictors of in vivo efficacy in a host or subject in need thereof (e.g., having or suspected of having at least one obligate anaerobe, or having or suspected of having an infection caused by or suspected of being caused by at least one obligate anaerobe). Accordingly, TABLES 1-9, 10A, 11 and 12, each represent co-agent combinations that may be useful as pharmacotherapy against an infection comprising or suspected of comprising an obligate anaerobe, in which the obligate anaerobe is a Gram-negative bacteria and/or a Gram-positive bacteria, including at least one or more of Clostridioides, Clostridium, Bifidobacterium, Peptostreptococcus, Propionibacterium, Cutibacterium Bacteroides, Parabacteroides, Phocaeicola, Prevotella, Porphyromonas, and/or Aggretibacter, and/or a microaerophilic Streptococcus (e.g., Streptococcus mutans), or any combination thereof. Because of the species and/or types of bacteria that co-agent combinations are antibacterially effective against, and because there is such high effectiveness in an anaerobic environment, the findings provide evidence that co-agent combinations herein not only provide a potentiation of one or more existing antibacterial co-agents, the co-agent combinations also broaden a spectrum of activity, for suitable antimicrobial activity and high efficacy against challenging anaerobes and/or antibiotic-resistant anaerobic pathogens, those that are considered causative of substantial and serious (complicated) infections. As such, co-agent combinations herein may be utilized as pharmacotherapy against a mixed infection (comprising one or more obligate anaerobes) and/or a complicated infection, such as but not limited to an infection identified elsewhere herein (e.g., located in an abscess, UT, respiratory tract, skin and skin/soft structures, intra-abdominally, as representative examples), and/or from a secondary infection (e.g., hospital-acquired infection, ventilator-associated infection, and/or in response to and/or following a serious or complicated viral infection, such as from SARS-CoV, HIV, and/or influenza virus, as representative examples). It is noted that in view of an incrementally higher synergistic action and synergism found herein (in which a five co-agent combination shows higher synergism, as lower FICI values, as compared with a four co-agent combination, and a four co-agent combination shows higher synergism, as lower FICI values, as compared with a three co-agent combination, which is not necessarily on a fixed scale), addition of a further co-agent (or two further co-agents or three further co-agents) to a three co-agent combination may allow for dose reduction (of at least one co-agent in a co-agent combination by) and/or a reduction in duration of dosing, either of which is beneficial for a host or subject in need, particularly when safety and/or side effects are a concern.

Importantly, in light of useful, valuable and highly operative synergy of co-agent combinations herein (whether three co-agents, four co-agents, five co-agents, or six co-agents), a further benefit of co-agent synergism and synergistic action, is an ability to increase exposure of at least one co-agent (or two co-agents, or three-co-agents, or four-agents, or five co-agents, or six co-agents) by providing said one co-agent, or two co-agents, or three co-agents, or four co-agents, or five co-agents, or six co-agents, respectively, above MIC of the combination, thereby achieving activity and antibacterial benefits from that one co-agent, or two co-agents, or three co-agents, or four co-agents, or five co-agents, or six co-agents, respectively, while ensuring synergy and synergistic action (as well as superior antibacterial efficacy) throughout a dosing schedule and/or dosing regimen.

Interestingly, as shown in TABLE 13, a co-agent combination, comprising at least F, D, S, is a selective antibacterial, and does not exhibit antibacterial activity against mutualistic or commensal Gram-positive facultative anaerobic bacteria (e.g., commensal under normal or non-diseased conditions), which include Lactobacillus spp., Pediococcus spp. and Leuconostoc spp. None of the following were inhibited by a co-agent combination when the combination was at least F, D, and S: Lactobacillus acidophilus, Lactobacillus delbrueckii, Lactobacillus gasseri, Lactobacillus paracasei, Lactobacillus plantarum, Leuconostoc mesenteroides, Pediococcus acidilactici or Pediococcus pentosaceus. Similar findings with other co-agent combinations were noticed (data not shown). It is possible that because such commensal and/or mutualistic Gram-positive facultative anaerobic bacteria are beneficial to a host (such as to a GI system), selectivity of co-agent combinations herein are purposeful, in which co-agents combinations herein facilitate antibacterial activity (such as any one or more of herein antibacterial responses (i)-(x)) against obligate anaerobic pathogens without presenting a general disruption of commensal and/or mutualistic Gram-positive facultative anaerobic bacteria, thereby maintaining at least part of GI tract microflora. Preservation of commensal/mutualistic Gram-positive facultative anaerobic bacteria of any one or more of which Lactobacillus, Pediococcus, and/or Leuconostoc can also be beneficial in preventing a secondary infection with a pathogenic bacteria. For example, the GI tract is predisposed to and subject to overgrowth and disease associated with Clostridioides (e.g., C. difficile) or Clostridium, when commensal microflora of the GI tract are inhibited, reduced in number, or eliminated. These benefits are best when pathogenic bacteria are targeted by at least F, D, S, or F, Q, S, and optionally a further co-agent (e.g., any one or more of PGSI, BLI, FMEI, when the pathogenic bacteria is not resistant or suspected of being resistant to the further co-agent). When resistance to the further co-agent is suspected or known, no further co-agent herein should be utilized.

Such unexpected finding shows that co-agent combinations have multiple beneficial effects that include: co-agent synergy and synergistic action when delivered to one or more obligate anaerobes; sufficient and/or effective antibacterial activity against and reduction or inhibition or elimination of one or more obligate anaerobes; lack of synergistic action when delivered to commensal and/or mutualistic Gram-positive facultative anaerobic bacteria selected from any one or combination of Lactobacillus, Pediococcus, and/or Leuconostoc; lack of antibacterial activity against commensal and/or mutualistic Gram-positive facultative anaerobic bacteria selected from any one or combination of Lactobacillus, Pediococcus, and/or Leuconostoc; and preservation of commensal and/or mutualistic Gram-positive facultative anaerobic bacteria selected from any one or combination of Lactobacillus, Pediococcus, and/or Leuconostoc.

TABLES 14A, 14B, and 15 confirm findings identified with at least TABLES 1-9, 10A, 11, and 12, in which synergistic action of co-agents and antibacterial activity of co-agent combinations are potentiated with inclusion of at least one further co-agent. TABLES 14A and 14B show unexpected improvements when a further co-agent is an FMEI (or inhibitor of a bacterial FME; here phosphonoformate). The FME, which is believed to mediate resistance to fosfomycin (i.e., by appending a glutathione moiety to an antibacterial fosfomycin and rendering it inactive), is not only unaffected by a three co-agent combination, a combination that does include F, but with inclusion of an FMEI, co-agent synergy is even further improved upon (see further shift and lower FIC values with the four co-agent combination), which is also reflected in better MIC values for at least D and S when the FMEI is included in the combination against both fosfomycin-susceptible and fosfomycin-resistant responsive Gram-positive and responsive Gram-negative facultative anaerobes in an aerobic environment. TABLES 14A and 14B show that a four co-agent combination (that includes an FMEI) is active against at least Staphylococcus aureus and Escherichia coli. Activity against S. aureus is particularly unexpectedly (and unpredicted) because S. aureus strains are not known to express FME with a glutathione moiety, nor have such FME inhibitors been found effective to date against S. aureus.

In TABLE 15 a further co-agent is a PGSI with or without a BLI, any of which potentiate synergistic action of co-agents as well as antibacterial activity of co-agent combinations when at least one further co-agent is included with a three co-agent combination of at least F, D, and S. Unexpected and better efficacy is found with a further co-agent being a PGSI, such as vancomycin or telavancin or meropenem. Better efficacy is also exhibited with a further co-agent when it is a PGSI and BLI, such as amoxicillin-clavulanate or ceftazidime-avibactam. TABLES 15 shows that a four co-agent combination (that includes a PGSI) as well as a five co-agent combination (that includes a PGSI and BLI) are all synergistic, having antibacterial activity against responsive facultative anaerobes (at least one or more of S. aureus, Enterococcus faecalis, E. coli, Klebsiella pneumoniae, Pseudomonas aeruginosa, and Acinetobacter baumannii in an aerobic environment). It is noted that with each of clavulanate the further PGSI and/or BLI co-agent is provided as described herein, an expected dosing for each will be at least at its approved dosing currently indicated for antibacterial efficacy.

TABLE 16 discloses a PAE with co-agents, F, D, S, which is at least as long as or longer than a co-agent, when provided singly or as a duo, against responsive facultative anaerobic pathogens in an aerobic environment. When PAE of co-agents, F, D, S, is as a long as PAE for an individual co-agent, it is noted that concentrations are not the same, such that with use of co-agents, F, D, S, (three co-agents), the concentration is ¼ of the amount used for an individual agent. And, when four or five co-agents are used, the amount of each co-agent used to achieve the PAE shown in the table is ⅛ to 1/16 less than the amount used for a comparable PAE with an individual co-agent. Thus, PAE with four co-agents (F, D, S, PGSI) or with five co-agents (F, D, S, PGSI, BLI) is at least as long as or longer than a co-agent provided singly or as a duo against responsive facultative anaerobic pathogens in an aerobic environment, while an amount of each co-agent in a combination is significantly less (e.g., ⅛ to 1/16 less) than an amount of an individual co-agent. PAE is a measure of a length of time that growth is inhibited after antibiotic absence, and is indicative of continued growth inhibition (and/or suppression and/or reduction and/or elimination of bacteria, and/or reduction or elimination of colony formation) after full life of a single dose or at a trough concentration in a host or subject receiving therapy. With each co-agent combination, TABLE 16 shows that PAE improvements are also synergistic, occurring even when a concentration of each co-agent in a co-agent combination is lower (or substantially lower) than the concentration used singly to show individual PAE. This is evidence of another powerful role of co-agent synergy, in which a co-agent combination sustains or continues growth inhibition (and/or suppression and/or reduction and/or elimination of bacteria, and/or reduction or elimination of colony formation) at a lower concentration of each co-agent when in a combination, the lower concentrations able to sustain or continue growth for at least a same length of time or for a longer time. Such findings are remarkable and unpredicted, as are the finding that while two co-agents as a duo (PGSI+BLI, here ceftazidime-avibactam) are used, there is no PAE (beta lactams do not exert a PAE). Yet, when these co-agents are with a three co-agent combination (F, D, S) against a responsive facultative anaerobes (E. coli), a PAE is observed, which is attributed, at least in part, to co-agent synergism, and synergistic action as identified unexpectedly herein. TABLE 16 shows that a four co-agent combination (which includes PGSI, here, as vancomycin) is sufficient not only as an effective antibacterial against beta-lactam- and methicillin-resistant S. aureus (at a lower concentration of each co-agent), but maintains PAE at much lower concentrations of each co-agent.

The clinical significance of finding that a co-agent combination herein (e.g., having at least F, D, S or F, Q, S) will likely extend a time period for growth inhibition (and/or suppression and/or reduction and/or elimination of bacteria, and/or reduction or elimination of colony formation), even after a level of co-agents has diminished below MIC, is its favorable impact by reducing resistant mutant selection at trough concentrations (such as prior to giving a dosing or between spaced apart dosings, e.g., spaced longer than a PAE). These findings are predictive of a low likelihood that resistance will develop with co-agent combinations and/or formulations herein, because lengthening PAE is related to or correlates with a lower likelihood of selected resistance due to exposure.

PAE was determined in-vitro by exposing a log-phase facultative anaerobic bacteria (growing overnight (≤12 hours) at 37° C. with shaking (˜200 rpm) in an aerobic environment in cation-adjusted Mueller-Hinton Broth (CAMHB), then diluting 1:6 in fresh pre-warmed CAMHB supplemented with 50 mg/L G6P, and incubating for 2 more hours) to various concentrations of co-agent(s) identified in TABLE 16 (2×, 4×, or 8×MIC) for one hour, followed by washing (to remove co-agent(s)), resuspending in pre-warmed growth medium, and monitoring each condition for a return to log growth. Aliquots from each condition were removed at time 0, and at 30-min intervals, ten-fold serially diluted, spread on Mueller-Hinton Agar plates, and incubated for 48 hours to allow for colony growth. Colonies were counted and multiplied by the inverse of the dilution to obtain each plate count and PAE value (plate counts were utilized to construct growth curves and to determine length of time required for one-log increase in bacterial count after removal of antibiotic). PAE was quantified by viable counts. PAE is defined by an equation: PAE=(number of hours for each culture condition to increase by one-log in cell number after removal of co-agent(s)) minus (number of hours for a no-co-agent control culture (otherwise treated identically) to increase one-log).

TABLE 17 provides an illustrative summary of susceptibility of certain obligate anaerobic tested with a single co-agent (F) or with a co-agent duo (D,S) or with a representative co-agent combination (F,D,S), each in an anaerobic environment. The table summarizes the fact that most GPOAs tested were susceptible (+) to F used singly, while not susceptible and were resistant (−) to the co-agent duo (D,S). Interestingly, Bifidobacterium was the GPOA that was generally susceptible to co-agent duo (D,S), instead having no susceptibility to F used singly. All GNOAs tested were not susceptible to F used singly, and were also not susceptible to the co-agent duo (D,S), with the exception of Porphyromonas gingivalis (ATCC 33277), which was susceptible to duo (D,S). And, despite the lack of activity of F and/or the co-agent duo (D,S) against obligate anaerobes, all obligate anaerobes tested were susceptible to at least a three co-agent combination (here F, D, S), the three co-agents being synergistic (as identified by FICI≤5), acting synergistically, which causes or promotes a sufficient and/or effective antibacterial activity by the co-agent combination (provided by at least growth inhibitory activity, and/or any of responses (i)-(x) described above) against each of the obligate anaerobes tested. The low calculated FIC values are also depicted in TABLE 17.

TABLES 18-21 are provided because it has been reported by others of skill in the field that pH may differ depending on a site of infection, and may be reduced or elevated from systemic homeostatic pH (which is at or near a neutral pH). For example, an abscess may be acidic (e.g., pH at about 5), skin may be acidic (e.g., pH at or about 5 or 6 or between about pH 5 to 6.5), while urine may have a pH anywhere between 5 and 8. Antibiotics often function poorly in acidic conditions. As such, a representative co-agent combination (here, F, D,S) against facultative anaerobes in an aerobic environment are tested for synergistic action and antibacterial activity at different pH conditions (pH 5, 7, 8, as shown in TABLES 18, 19, and 20, respectively), and compared with data when a co-agent is used singly (F) or as a duo (D,S). In general, regardless of pH condition (acidic (pH 5) or neutral (pH 7) or basic (pH 8)), many Gram-positive facultative anaerobes were responsive to F alone, while more Gram-negative facultative anaerobes (e.g., certain P. aeruginosa, K. pneumoniae, and Burkholderia thailandensis) were resistant to F alone. Only two Gram-negative strains (Acinetobacter baumannii 918857, Burkholderia thailandensis ATCC 700388) were compromised at the basic pH, with no growth at pH 8. E. faecium (Gram-positive) and several types and/or strains of Gram-negative facultative anaerobes showed resistance to the duo (D,S) at pH 5. More Gram-positive and Gram-negative facultative anaerobes were resistant to D,S (e.g., all strains of P. aeruginosa) at neutral or basic pHs. On the other hand, and unlike many currently approved antibiotics, regardless of a change in pH, co-agent combinations (comprising at least F, D, S) maintained their synergistic action throughout the pH range, being surprisingly active as an antibacterial against all the facultative anaerobes tested at acidic pH, as well as neutral and basic pH conditions (in aerobic environment), with the exception of the two compromised Gram-negative strains that did not grow at pH 8. Consistently, as shown in TABLE 21, MIC values of the co-agent combination were remarkably low at all pH conditions against all the facultative anaerobes tested (those considered to be responsive facultative anaerobes, and not the non-responsive or mutualistic and/or commensal facultative anaerobes of any of Lactobacillus spp., Leuconostoc spp. and Pediococcus spp.). Thus, while the tables show that many of the responsive facultative anaerobes tested are drug-resistant or multi-drug resistant strains (resistant to one or more existing, approved antibiotics and/or to at least one of F, D, and/or S), synergy with co-agents under all tested pH conditions (e.g., pH 5 to 8) were observed, which is any pH in which an anaerobic pathogen may be growing, regardless of tissue or fluid or pathologic condition. TABLE 21 shows that at all pH conditions, a co-agent combination herein remains synergistic, being active and effective against responsive anaerobes (here, responsive facultative anaerobes), including drug-resistant strains and strains tested and found resistant to at least one co-agent when used singly (F) or as a duo (D, S). A similar finding is considered likely with difficult to treat anaerobes or with obligate anaerobes.

Taken together, the tables and accompanying information herein show that co-agent combinations and/or formulations herein are active as antimicrobials (e.g., comprising at least F, D, S, or comprising at least F, Q, S) and exhibit at least the following characteristics: synergistic action, providing, as synergists, remarkably low (or better) MIC for effective antibacterial activity against obligate anaerobes; synergistic action, providing, as synergists, remarkably low MIC for effective antibacterial activity against responsive facultative anaerobes; synergistic action, providing, as synergists, remarkably low MIC for effective antibacterial activity against obligate anaerobes in a mixed population; and no synergy or antibacterial activity against mutualistic and/or commensal (non-responsive) facultative anaerobes consisting of Lactobacillus spp., Leuconostoc spp. and/or Pediococcus spp., in which such characteristics cause or promote any one or more or any reasonable combination of actions (i)-(x) described above, such as growth inhibitory activity and/or growth suppressing activity, and/or reduction and/or destruction and/or elimination of bacteria, and/or reduction and/or prevention and/or elimination of colony formation. Co-agent combinations and/or formulations herein are selectively synergistic, and had no observed antibacterial activity (e.g., no growth inhibitory activity) against Lactobacillus spp., Leuconostoc spp. and Pediococcus spp., which was not only unexpected, but also not predicted. Pharmaceutically acceptable co-agents in one or more co-agent combinations and/or formulations herein exhibit synergy and are antimicrobially effective in an anearobic environment against pathogenic anaerobes, including obligate anaerobes.

Information herein shows that a three co-agent combination is truly beneficial and superior to many existing treatments currently utilized against more difficult to treat pathogens, obligate anaerobes, because of co-agent synergy that provides synergistic action, causing or promoting effective antimicrobial activity (with remarkably low MICs) against even the more difficult to treat obligate anaerobes. By including a further co-agent to a three co-agent combination, co-agent synergism is amplified and MICs are further reduced when tested against the difficult to treat obligate anaerobes. Importantly, said actions and activities occur in an anaerobic environment, showing versatility and valid utility of co-agent combinations and/or formulations herein (having three or more co-agents described herein). The evidence and data support a combination pharmacotherapy approach using co-agent combinations herein over a mixing strategy (random treatment of half of subjects or patients with a first antibiotic and the other half with a different antibiotic) or a cycling strategy (temporal rotation of treatment, each antibiotic at a different period), which are two strategies often performed in a hospital setting. Importantly, co-agent synergy as identified herein offers a large advantage, not only in allowing for a possible reduction in concentration of at least one co-agent when safety is a concern, but, in particular, by causing sufficient and/or effective activity (of the co-agent combination and/or formulation) against resistant anaerobic strains (because, as shown herein, resistant strains, whether acquired, intrinsic, or expected phenotypic resistance, against one drug or multi drugs, remain susceptible to herein co-agent combinations), and by providing such enhanced antibacterial activity in an anaerobic environment, and by amplifying co-agent efficacy, as well as by lengthening PAE, which should inevitably reduce SMF cases. Inventors determined that SMF to a three co-agent combination (F,D,S) against strains of responsive facultative anaerobes (E. coli, S. aureus, and E. faecalis) was at least two order of magnitude or 100-fold less frequent as compared with a single co-agent (F) or a duo (D,S) against the same strains, and findings occurred even when the co-agent (F or D,S) was tested at a higher concentration.

Observations and findings by inventors are highly suggestive of and provide a high expectation of killing of obligate anaerobes and facultative anaerobes responsive to co-agent combinations and/or formulations herein, which excludes non-responsive and mutualistic and/or commensal facultative anaerobes of Lactobacillus spp. or Leuconostoc spp. or Pediococcus spp. And, with even lower MIC and FIC values when more than three co-agents were utilized and administered to tested anaerobes, which were the obligate anaerobes and facultative anaerobes responsive to co-agent combinations and/or formulations herein, and were not the non-responsive or mutualistic and/or commensal facultative anaerobes of Lactobacillus spp. or Leuconostoc spp. or Pediococcus spp., bactericidal activity is likely when any of herein co-agent combinations and/or formulations are delivered in pharmaceutically sufficient amounts as an effective antibacterial against obligate anaerobes and facultative anaerobes responsive to such antimicrobial co-agents.

Combinations and/or formulations herein are a plurality of co-agents (e.g., three co-agents, four co-agents, five co-agents, six co-agents), the co-agents being provided in one or more pharmaceutically acceptable forms, in which co-agents are in an effective amount for synergistic action, co-agents being provided via one or more delivery and/or dispensing means as effective pharmacotherapy, such as in an anaerobic environment. The plurality of co-agents may be in a same formulation, or in one or more different formulations. The plurality of co-agents may be in a same vehicle or conveyance for delivery and/or dispensing, or in one or more different vehicles or conveyances for delivery and/or dispensing. A dispensing or delivery means is a method or mode of providing or administering combinations and/or formulations herein, including but not limited to intravenous, intramuscular, oral, buccal, dental, cutaneous, subcutaneous, endocervical, endosinusial, endotracheal, epidural, enteral, intra-abdominal, intracerebral, intradermal, intraspinal, intrauterine, nasal, parenteral, rectal, sublingual, vaginal, as representative examples. Co-agents for delivery and/or dispensing are often provided in a same vehicle or conveyance, unless, e.g., one or more co-agents, such as in a kit, are individualized and later delivered or dispensed in a same formulation or by a same or similar means of delivery and/or dispensing. Vehicles or conveyances available for co-agents herein include pill, tablet, capsule, gel, lozenge, aerosol, spray, liquid, suspension, sachet, bag, suppository, vial, implant, lotion, cream, ointment, as representative examples for purposes herein. In at least one embodiment, a plurality of co-agents are in a same type or similar type of vehicle, the vehicle being any vehicle or conveyance described and/or contemplated herein. In at least one embodiment, a plurality of co-agents are at least some or all being in a same type or similar type of vehicle, the vehicle being any vehicle or conveyance described and/or contemplated herein, and when in a same type of vehicle, some or all co-agents may be compartmentalized, commingling when delivered or dispensed in or on a subject in need thereof. In at least one embodiment, at least two co-agents are in a same vehicle, the vehicle being any vehicle or conveyance described and/or contemplated herein. In at least one embodiment, at least three co-agents are in a same vehicle, the vehicle being any vehicle or conveyance described and/or contemplated herein. In at least one embodiment, at least four co-agents are in a same vehicle, the vehicle being any vehicle or conveyance described and/or contemplated herein. In at least one embodiment, at least five co-agents are in a same vehicle, the vehicle being any vehicle or conveyance described and/or contemplated herein. In at least one embodiment, at least six co-agents are in a same vehicle, the vehicle being any vehicle or conveyance described and/or contemplated herein. In one or more embodiments, co-agents are in a same pharmaceutically acceptable formulation. In one or more embodiments, co-agents are in different pharmaceutically acceptable formulation and in a same vehicle (e.g., in one or more layers or coats or films or sections or partitions, as examples). All co-agents may be formulated and in a same aerosol or spray, either in a same preparation or one or more different preparations (e.g., in one or more particles or solids or droplets). All co-agents may be formulated and in a same liquid/fluid or suspension, either in a same preparation or one or more different preparations (e.g., in one or more concentrates). All co-agents may be formulated in a same sachet or bag or suppository or vial or implant, either in a same preparation or one or more different preparations (e.g., in one or more sections or partitions or layers). In one or more embodiments, all co-agents may be formulated in a same lotion or cream or ointment or balm, either in a same preparation or one or more different preparations (e.g., in one or more emulsions or layers). In addition, or as an alternative, at least one co-agent may be formulated in a separate pill or tablet or capsule or gel or lozenge. In addition, or as an alternative, at least one co-agent may be formulated in a separate aerosol or spray. In addition, or as an alternative, at least one co-agent may be formulated in a separate liquid/fluid or suspension. In addition, or as an alternative, at least one co-agent may be formulated in a separate sachet or bag or suppository or vial or implant. In addition, or as an alternative, at least one co-agent may be formulated in a separate lotion or cream or ointment or balm. As such, any one of the vehicles or conveyances may have or contain one or more co-agents. In one or more embodiments, any one of the vehicles or conveyances may have or contain all co-agents. In one or more embodiments, any one of the vehicles may be compartmentalized, containing one or more co-agents in a compartment or layer or partition or section or film or emulsion or suspension or solid or particulate. In one or more embodiments, any one of the vehicles may having one or more co-agents therein, each or some co-agents being independent and/or independently formed (e.g., independent beads or grains or liposomes or colloids or particles or nanoparticles or solids or emulsions or films or layers).

Generally, a co-agent composition and/or formulation herein (e.g., pharmaceutical or veterinary) is formulated for a route of administration (e.g., systemic delivery or dispensing means) selected from topical, transdermal, enteral, oral, subcutaneous, enema, rectal, inhalation, parenteral, intranasal, intravenous, intramuscular, intraperitoneal, and/or intraocular, as representative examples. In addition, or alternatively, a composition and/or formulation herein (pharmaceutical or veterinary) when formulated will comprise one or more pharmaceutically acceptable excipients (e.g., inactive ingredient(s)) suitable with an acceptable manufacturing practice and process for pharmacotherapy medicaments, and for a desired means of delivery or dispensing. Excipients may promote one or more of binding, stability, density, texture, taste, and/or release. A co-agent composition and/or formulation herein (e.g., having one or more pharmaceutically acceptable excipients) when formulated will be provided to a host or in accordance with its formulation (e.g., with or without a diluent or other ingredient, for systemic delivery). In some forms, administration is by at least an intravenous/injection route. In some forms, administration is by at least an oral or inhalation route. In some forms, administration is by at least a parenteral route. In some forms, administration is by at least a suppository. Administration may be by at least an enema or retentate enema.

For solid or liquid compositions and/or formulations in one or more pharmaceutically acceptable forms, the vehicle or conveyance may include but not limited to tablet, capsule, caplet, powder, granules, beads, liquid, suspension, solution, emulsion, syrup, elixir, drops/droplets, semi-solid (e.g., waxinate, gum, gummy, cream, lotion, balm, suppository), any of which may be ready-to-use or a concentrate. In some forms, a solid or liquid may be a concentrate (dry or liquid or semi-solid), which is later solubilized, re-suspended and/or diluted, usually prior to administration, using sterile water, saline, particulates, powder, or any other appropriate sterile medium known in the art for solubilizing, re-suspending and/or diluting. Nontoxic solid carriers and/or diluents may be included in any forms herein, represented by at least a pharmaceutical grade of mannitol, lactose, starch, magnesium stearate, sodium saccharine, talcum, cellulose, glucose, sucrose, magnesium, carbonate, and the like. One or more binders may be included (e.g., for compressed form or tablet), including ones that impart a cohesive quality to a powder; binders are represented by at least a pharmaceutical grade of starch, gelatin, one or more sugars (e.g., lactose, dextrose), natural gum, synthetic gum. One or more disintegrants may be included that facilitate break-up; disintegrants are represented by at least a pharmaceutical grade of starch, clay, cellulose, algin, gum, crosslinked polymer. One or more lubricants and/or glidants may be included that prevent adhesion to surface (e.g., in a manufacturing process and/or with administration and/or to improve flow characteristics for manufacturing and/or administration). Glidants are represented by at least a pharmaceutical grade of colloidal silicon dioxide. Lubricants are represented by at least a pharmaceutical grade of talc, stearic acid.

A solid or liquid may or may also include, as examples, solubiliser, emulsifier, buffer, antimicrobial preservative, sweetener, flavoring agent, suspending agent, thickening agent, colorant, viscosity regulator, stabilizer and/or osmo-regulator. A liquid for enteral administration may include water (containing one or more additives, such as cellulose (or derivatives thereof), sodium carboxymethyl cellulose solution, alcohol (e.g., monohydric alcohols, polyhydric alcohols, glycols), and/or oils (e.g. fractionated coconut oil and arachis oil). A liquid for parenteral administration may include an oily ester (e.g., ethyl oleate, isopropyl myristate). For oral and/or inhalation, other solutes and/or suspending agents and/or oils and/or fats may or may also be included (e.g., enough saline and/or glucose, and/or other additives to make a solution or suspension isotonic, and/or any one or more of at least a pharmaceutical grade of a salt, phosphate, gelatin, oleate ester of sorbitol and/or its anhydride copolymerized with ethylene oxide (e.g., sorbitan monoleate, polysorbate 80).

For topical and/or transdermal compositions and/or formulations in one or more pharmaceutically acceptable forms (e.g., semi-solid, waxinate, cream, gel, foam), the vehicle or conveyance may include but is not limited to ointment, cream, lotion, balm, and gel. One or more appropriate penetrants and/or detergents may be included that facilitate permeation, represented by a pharmaceutical grade of at least dimethyl sulfoxide, dimethyl acetamide, and dimethylformamide.

For transmucosal compositions and/or formulations in one or more pharmaceutically acceptable forms (e.g., liquid, suspension, emulsion, solid, semi-solid, particles), the vehicle or conveyance may also include but is not limited to nasal spray, oral spray, retentate enema, rectal and/or vaginal suppository. One or more base compounds may be included of the type utilized transdermally, as are known to one of skill in the art, represented by at least a pharmaceutical grade of cocoa butter, polyethylene glycol (carbowax), polyethylene sorbitan monostearate, or mixtures thereof, with or without other materials that modify melting point and/or dissolution rate, which are known to one of skill in the art.

A co-agent composition and/or formulation when formulated may be prepared according to methods known in the art that release one or more ingredients in the formulation substantially upon or soon after administration. In one or more forms, any co-agent composition and/or formulation when formulated may be prepared according to methods known in the art that release ingredients in the formulation at a predetermined time and/or predetermined period after administration and/or predetermined location on or in a host or subject in need thereof. Methods for controlling or inhibiting or destroying or eliminating one or more pathogenic anaerobes or its colony formation in or on a host or subject may comprise: providing or delivering or dispensing (for administration) an effective or therapeutically effective amount of a co-agent combination comprising a plurality of co-agents, being at least three active co-agents herein, to the host or subject, the co-agents exhibiting synergy, synergy causing or promoting antimicrobial activity, the co-agents being effective to cause or promote at least a response in one or more pathogens, the response being selected from one or more of: (a) inhibiting growth of one or more pathogenic anaerobes; (b) inhibiting growth of one or more pathogenic anaerobes for a sustained period; (c) suspending growth of one or more pathogenic anaerobes; (d) eliminating in whole or in part one or more pathogenic anaerobes; (e) destroying in whole or in part one or more pathogenic anaerobes; (f) interfering in whole with one or more pathogenic anaerobes; (g) altering and/or enhancing and/or affecting and/or reversing susceptibility of one or more pathogenic anaerobes which is not susceptible and/or is intrinsically resistant to at least one co-agent when utilized singly (or as a duo) against one or more pathogenic anaerobe. Pathogenic anaerobes may be in an anaerobic environment.

In some embodiments is a method for personalized pharmacotherapy or treatment for a host or subject (e.g., an individual) in need thereof for an anaerobic bacteria or infection caused by or suspected of being caused by the anaerobic bacteria, the method comprising: i) obtaining a biologic sample from the host or subject and identifying at least one anaerobic bacteria suspected of being a pathogen in the biologic sample; and ii) providing or delivering or dispensing to the host or subject pharmaceutically acceptable co-agents comprising a plurality of co-agents being at least three active co-agents in a same or different formulation. Here, providing or delivering or dispensing may be at a same or similar time or at different times within a period that is up to about or less than about two hours. Here, providing or delivering or dispensing may be continuous. The biologic sample may comprise pathogenic and/or non-pathogenic anaerobic bacterial types and/or species, and following the providing or delivering or dispensing, at least one pathogenic anaerobe (and/or colony thereof) on or in the host or subject is inhibited or eliminated or controlled, while a sufficient amount of non-pathogenic bacterial types and/or species (and/or colony thereof) are not sufficiently compromised.

In some embodiments is a pharmaceutically acceptable medicament comprising a plurality of co-agents in a same or different formulation for use as pharmacotherapy for a host or subject having or suspected of having one or more pathogenic anaerobes, and is an antimicrobial being effective against the one or more pathogenic anaerobes, wherein at least some pathogenic anaerobes are in an anaerobic environment, wherein the plurality of active co-agents are at least three co-agents in use as the pharmacotherapy (e.g., F,D,S, or F, Q, S), being synergistic, causing effective antimicrobial activity as compared with an active co-agent (singly or as a duo), the co-agent (singly or as duo) less effective or ineffective when provided singly or as the duo against the one or more pathogens (which may be identified by in vitro AST and by calculation of FICI). At least one active co-agent in use as pharmacotherapy is a synergist, for promoting or causing synergistic action with other active co-agents in use as the pharmacotherapy, inducing a reversal effect on at least one co-agent (e.g., the co-agent being inactive or ineffective when provided singly as a duo against the one or more pathogens, which may be identified by in vitro AST and by calculation of FICI). At least one active co-agent in use as the pharmacotherapy is a synergist, for promoting or causing synergistic action with other active co-agents in use as the pharmacotherapy, inducing a reversal effect on resistance to the at least one co-agent against one or more pathogenic anaerobes (e.g., reversal of resistance to the at least one active co-agent when used singly or as a duo against the one or more pathogenic anaerobes). The plurality of co-agents or medicament may be provided empirically, or after an in vitro testing.

In some embodiments is an in-situ synergy of a plurality of co-agents, in use as pharmacotherapy as an antimicrobial against a target comprising one or more pathogenic anaerobes, being delivered or dispensed to a host or subject in need in a period, and in a same or different formulation, the in situ synergy promoting or causing or eliciting a response, the response comprising one or more or any combination of: (a) reducing growth of the target; (b) inhibiting growth of the targeted; (c) suspending growth of the target; (d) eliminating the target in whole or in part; (f) interfering metabolically with the target in whole or in part; and (g) altering or enhancing or promoting or reversing susceptibility of the target, in which the target may or may not be resistant (acquired, intrinsic or phenotypically) to at least one active co-agent of the plurality of co-agents when the at least one active co-agent is used singly against the target, and in which the period is one or more of less than or within about 1 hour or less than or within about two hours or less than or within about 3 hours. In some embodiments, the target is one or more of at least one GNOA bacteria, at least one GPOA bacteria, at least one GNOA bacteria in a mixed population, at least one GPOA bacteria in a mixed population, and any combination thereof. The target may be on or may reside in an anaerobic environment. In some embodiments, more than one of the plurality of co-agents, being delivered or dispensed, is by a same means of delivery or dispensing, said means being any one or more of the routes of delivery or dispensing described herein. In some embodiments, more than one of the plurality of co-agents, being delivered or dispensed, is by a different means of delivery or dispensing, said means being any one or more of the routes of delivery or dispensing described herein. The host or subject has or is or suspected of having the target in or on the target. The host or subject may have an infection caused by or suspected of being caused by the target, or may be at risk of developing an infection caused by or suspected of being caused by the target, or may have a disorder and/or a disease due to suspected of being due at least in part to the target. Diagnostic and/or analytic methods, including in vitro methods, for or involving the target, the infection, the disorder and/or the disease will be and/or are well known by a person of skill in the relevant art for diagnosing or determining and/or analyzing the target. In one or more embodiments, the target, the infection, the disorder and/or the disease presents or is suspected of being resistant to treatment or to prior pharmacotherapy. In some embodiments, the target, the infection, the disorder and/or the disease presents or is suspected of presenting as antibiotic resistant (to one or more currently approved antibiotics). In one or more embodiments, the target, the infection, the disorder and/or the disease presents or is suspected of presenting as resistant to at least one co-agent (resistant when tested independently or singly against the target). In some embodiments, the host or subject has never received treatment prior to the more than one active co-agent being delivered or dispensed. In some embodiments, the host or subject has received at least one treatment prior to the more than one active co-agent being delivered or dispensed. In some embodiments, the more than one active co-agents are delivered or dispensed daily until the target and/or the infection and/or the disorder and/or the disease is inhibited and/or abated and/or controlled and/or eliminated or resolved to a clinically acceptable level for that target and/or the infection and/or the disorder and/or the disease (e.g., per established clinical and/or hospital guidelines). In some embodiments, the more than one active co-agents are delivered or dispensed in a period and on a daily schedule being one time per day or two times per day or three times per day or four times per day or five times per day or six times per day. In some embodiments, the more than one active co-agents are delivered or dispensed in a period and continuously. In some embodiments, the more than one active co-agents are delivered or dispensed in a period and on a daily schedule until or as long as the target anaerobe, the infection, the disorder and/or the disease (suspected or otherwise) persists or is considered for treatment or resolves. The more than one active co-agents may be at least three co-agents herein (at least F, D, S, or at least F, Q, S), or are at least four co-agents herein (comprising at least F, D, S, or comprising at least F, Q, S, with any one of PGSI, BLI, or FMEI), or are at least five co-agents herein (comprising at least F, D, S, or comprising at least F, Q, S, with any two of PGSI, BLI, and/or FMEI), or are at least six co-agents herein (comprising at least F, D, S, or comprising at least F, Q, S, with any three of PGSI, BLI, and/or FMEI).

In any embodiments described herein, a plurality of co-agents (also referred to as active co-agents and/or three or more co-agents) may have a same daily dosing schedule, or a similar daily dosing schedule, or an overlapping daily dosing schedule, and/or its own dialing dosing schedule, each being delivered or dispensed in the period (e.g., any one or more of less than or within about 1 hour or less than or within about 2 hours or less than or within about 3 hours), and may be in a same form or in a different form (e.g., in accordance with a means or route for delivery or dispensing). In any embodiments herein, co-agents are delivered via at least one vehicle described, which is a same vehicle or a different vehicle for each co-agent, and in a form for delivery that is either a same form or a different form (e.g., one or more of solid form or liquid form or semi-solid form and/or dry or wet). With one or more means or routes for delivery or dispensing of the plurality of co-agents (e.g., three or four or five or six co-agents described herein), co-agents in effective amounts are provided to a host or subject in need thereof by said one or more means or routes for delivery or dispensing (e.g., intravenous, intramuscular, oral, buccal, dental, cutaneous, subcutaneous, endocervical, endosinusial, endotracheal, epidural, enteral, intra-abdominal, intracerebral, intradermal, intraspinal, intrauterine, nasal, parenteral, rectal, topical, sublingual, vaginal, as representative examples), said effective amounts being sufficient for synergy and synergistic action as well as for effective antibacterial activity against one or more type and/or species of herein pathogenic anaerobes. In some embodiments, said effective amounts will also exhibit or elicit or cause as a response any one or more of (a) to (g) herein in an anaerobic environment. Yet, the plurality of co-agents remain selective by having no synergy and, thereby having no antibacterial activity and no inhibitory activity (nor any synergistic action) against certain mutualistic and/or beneficial facultative anaerobes (e.g., commensal bacteria in a normal or indigenous or non-diseased microflora) that are any one or combination of Lactobacillus spp., Leuconostoc spp. and Pediococcus spp.).

Because some infections, such as in a host or a subject, have higher numbers of pathogenic bacterial cells which can lead to an inoculum like effect, the MIC may be elevated in the presence of higher bacterial load, thus more drug may be required. Such a scenario is more likely in complicated or serious (severe) infections. In a clinical setting, a higher antibiotic exposure is better, particularly for a complicated or serious infection. For example, serious infections necessitating IV administration, may have a higher initial dosing schedule to increase surety of clinical cure, and lead to a reduced length of hospital stay. As such, in one or more embodiments, dosing of one or more co-agents for co-agent combinations and/or formulations herein are greater than MIC, or significantly greater than MIC, for high co-agent exposure to one or more herein pathogenic anaerobes (e.g., being or including one or more herein obligate anaerobes). Fortunately, co-agents described herein have good tissue penetration, and, because of the unexpected but beneficial co-agent synergism described herein, even more difficult or serious infections (e.g., complicated UTI and/or pyelonephritis, as examples), may be treated with co-agents at acceptable and sufficient co-agent levels and/or with much higher than MIC co-agent levels that promote antimicrobial activity (e.g., any one or more of activities (i)-(x)) without drug toxicity. It is also understood that in some embodiments, a certain level of reversible toxicity may be acceptable because of a shorter duration of use provided herein, which is due to the benefit of synergistic action of co-agents in combinations and/or formulation herein.

Exemplary and representative dosing schedules are described, in which a plurality of co-agents herein (e.g., three or more co-agents and up to six co-agents) are either in a same formulation or two or more separate formulations, any of which may be provided in a same or different mode or means of delivery or dispensing, and which may be provided at a same time or period, or at substantially a same or similar time or period or one or more times in a period (such as within three hours, within two hours, within one hour, within 30 min, within 15 min, as representative examples) or continuous. Delivery or dispensing of the plurality of co-agents in a period warrants an overlapping delivery or dispensing for synergy and synergistic action. For such a plurality of co-agents herein, overlapping periods of delivery or dispensing ensures a response (e.g., any one or more of responses (a) to (g)). For such a plurality of co-agents herein, overlapping periods of delivery or dispensing ensures sufficient and/or effective antimicrobial activity (e.g., any one or more of activities (i)-(x)). For purposes herein, overlapping periods of delivery or dispensing may also be referred to as co-dosing.

In embodiments herein, delivery or dispensing of a pharmaceutically acceptable combination and/or formulation herein includes providing a plurality of co-agents (e.g., comprising at least three active co-agents herein or at least four active co-agents herein or at least five active co-agents herein or at least six active co-agents herein in a same or more than one different forms and/or formulations) at amounts for synergy and/or antibacterial activity in an anaerobic environment, the providing being at a same time or substantially a same or similar time or in a period (such as within about three hours, within about two hours, within about one hour, within about 30 min, within about 15 min, as representative examples) or continuously. For sufficient and/or effective antibacterial activity of the plurality of co-agents, the plurality of co-agents will comprise in 24 hours at least one dosing of F, D, S or F, Q, S (in a same or different form and/or formulation), and optionally with a therapeutically effective amount of any of FMEI, PGSI, and/or BLI. The at least one dosing in 24 hours may be provided once, twice, three times, four times, six times, eight time or twelve times, and may be only a one day dosing, or may be extended for several days or weeks and/or until discontinuation is appropriate. Generally, for synergy and/or antibacterial activity, an amount of F is a minimum of about 2.4 g and up to about 36 g (or any amount in a range therebetween, in 24 hours), and an amount of D (or Q) is a minimum of about 130 mg and up to about 1110 mg (or any amount in a range therebetween, in 24 hours), and an amount of S is a minimum of about 650 mg and up to about 5600 mg (or any amount in a range therebetween, in 24 hours). In many instances, and in view of the remarkable synergy disclosed herein that benefits exposure above the MIC of co-agents herein, dosing strategies may include higher doses of one or more of the plurality of co-agents. For example, in 24 hours, an amount for sufficient and/or effective synergy and antibacterial activity, co-agent F may be from about 20 g to about 36 g (or in any range therein), and co-agent D (or Q) may be from about 500 mg to about 1110 mg (or in any range therein), and co-agent S may be from about 2000 mg to about 5600 mg (or in any range therein). In 24 hours, a maximum amount of F may be at about or up to about 36 g, a maximum amount of D (or Q) may be at about or up to about 1110 mg, and a maximum amount of S may be at about or up to about 5600 mg, which are all historically safe amounts.

For sufficient and/or effective pharmacotherapy (e.g., for synergy and/or antimicrobial efficacy) any of FMEI, PGSI, and/or BLI, when included, is provided in a clinical- or hospital-approved daily amount and/or schedule. Vancomycin (a PGSI) is often provided as at or up to about 500 mg (IV) every six hours (four times daily) or at or up to about 1 g (IV) every twelve hours (twice daily), and would be beneficial when included in a four times daily or twice daily schedule with F, D, S or F, Q, S. Meropenem (a PGSI) is often provided up to or at about 2 (IV) every eight hours (three times daily), and would be beneficial when included in a three times daily schedule with F, D, S or F, Q, S. Meropenem-vaborbactam (a PGSI-BLI duo) is often provided up to or at about 4 g (IV) every eight hours (three times daily), and would be beneficial when included in a three times daily schedule with F, D, S or F, Q, S. Imipenem-elebactam-cilastatin (a PGSI-BLI trio) is often provided up to or at about 1.5 g (IV) every six hours (four times daily), and would be beneficial when included in a four times daily schedule with F, D, S or F, Q, S. Amoxicillin-clavulanate (a PGSI-BLI duo) is often provided up to or at about 1 g (total, IV) every eight hours (three times daily) or up to about 500 mg (total, oral) every eight hours or up to about 2 g (total, oral) every twelve hours (twice daily), and would be beneficial when included in a two times or three times daily schedule with F, D, S or F, Q, S. Ceftazidime-avibactam (a PGSI-BLI duo) is often provided up to or at about 2.5 g (total, IV) every eight hours (three times daily), and would be beneficial when included in a three times daily schedule with F, D, S or F, Q, S. Ceftolozane-tazobactam (PGSI-BLI duo) is often provided up to or at about 1.5 g (total, IV) every eight hours (three times daily), and would be beneficial when included in a three times daily schedule with F, D, S or F, Q, S. Ticarcillin-clavulanate (a PGSI-BLI duo) is often provided up to or at about 3.2 g (total, IV) every four to six hours (six or four times daily), and would be beneficial when included in a four times or six times daily schedule with F, D, S or F, Q, S. Ampicillin-sulbactam (a PGSI-BLI duo) is often provided up to or at about 3 g (total, IV or IM) every six hours to eight hours (four times or three times daily), and would be beneficial when included in a four times or three times daily schedule with F, D, S or F, Q, S. Piperacillin-tazobactam is often provided up to or at about 4.5 g (total, IV or IM) every six hours to eight hours (four times or three times daily), and would be beneficial when included in a four times or three times daily schedule with F, D, S or F, Q, S. Pre-made and non-pre-made duos or trios of PGSI, BLI, and/or FMEI may be utilized in accord with a clinical and/or hospital practice and would be beneficial when provided in accord with their daily schedule and when co-dosing or continuously dosing with a plurality of co-agents comprising at least F, D, S or at least F, Q, S.

In some embodiments, a daily co-dosing (in any amount in said ranges) is provided once daily or is calculated to provide co-agents in more than one dose (co-dose) per day (e.g., twice daily or thrice daily or four times daily or six times daily or eight times daily or twelve times daily, in any amount in said ranges) or is calculated to provide co-agents continuously (in any amount in said ranges). In some embodiments, a daily co-dosing is an amount for sufficient and/or effective pharmacotherapy against one or more pathogenic anaerobes in an anaerobic environment, such as in a host or subject or against an infection having or suspected of having the one or more pathogenic anaerobes, and is or includes a sufficient and/or effective amount of F and a sufficient and/or effective amount D (or Q) and a sufficient and/or effective amount S. In some embodiments, a daily co-dosing for antimicrobial pharmacotherapy is or includes at least F as a once daily amount of at or about 2.4 g and up to about 36 g, and D (or Q) as a once daily amount of at or about 130 mg and up to about 1110 mg, and S as a once daily amount of at or about 650 mg and up to about 5600 mg. In other embodiments, a daily co-dosing for antimicrobial pharmacotherapy is or includes at least F as a twice daily amount of at or about 1.2 g and up to about 18 g (per dose, two doses/day), and D (or Q) as a twice daily amount of at or about 65 mg and up to about 555 mg (per dose, two doses/day), and S as a twice daily amount of at or about 325 mg and up to about 2800 mg (per dose, two doses/day). In further embodiments, a daily co-dosing for antimicrobial pharmacotherapy is or includes at least F as a thrice daily amount of at or about 0.8 g and up to about 12 g (per dose, three doses/day), and D (or Q) as a thrice daily amount of at or about 43 mg and up to about 370 mg (per dose, three doses/day), and S as a daily amount of at or about 215 mg and up to about 1870 mg (per dose, three doses/day). In further embodiments, a daily co-dosing for antimicrobial pharmacotherapy is or includes at least F as a four times daily amount of at or about 0.6 g and up to about 9 g (per dose, four doses/day), and D (or Q) as a four times daily amount of at or about 32 mg and up to about 280 mg (per dose, four doses/day), and S as a four times daily amount of at or about 162 mg and up to about 1400 mg (per dose, four doses/day). In further embodiments, a daily co-dosing for antimicrobial pharmacotherapy is or includes at least F as a six times daily amount of at or about 0.4 g and up to about 6 g (per dose, six doses/day), and D (or Q) as a six times daily amount of at or about 21 mg and up to about 185 mg (per dose, six doses/day), and S as a six times daily amount of at or about 108 mg and up to about 935 mg (per dose, six doses/day). In further embodiments, a daily co-dosing for antimicrobial pharmacotherapy is or includes at least F as an eight times daily amount of at or about 0.3 g and up to about 4.5 g (per dose, eight doses/day), and D (or Q) as an eight times daily amount of at or about 16 mg and up to about 139 mg (per dose, eight doses/day), and S as an eight times daily amount of at or about 81 mg and up to about 700 mg (per dose, eight doses/day). In further embodiments, a daily co-dosing for antimicrobial pharmacotherapy is or includes at least F as a twelve times daily amount of at or about 0.2 g and up to about 3 g (per dose, twelve doses/day), and D (or Q) as a twelve times daily amount of at or about 10 mg and up to about 92 mg (per dose, twelve doses/day), and S as a twelve times daily amount of at or about 54 mg and up to about 468 mg (per dose, twelve doses/day).

A plurality of active co-agents may comprise at least an F, a D, and an S (each singly active against an aerobic bacteria in an aerobic environ, and at least one being singly inactive against an anaerobe in an anaerobic environ), are co-dosed daily every four hours or every six hours (e.g., parenterally), F being from about 1.8 to about 7.5 grams (per dose), D being about 5 to about 20 mg/kg (per dose), and S being about 20 to about 100 mg/kg (per dose). Active co-agents may comprise at least an F, a D, and an S (each singly active against an aerobic bacteria in an aerobic environ, and at least one being singly inactive against an anaerobe in an anaerobic environ), are co-dosed daily every four hours or every six hours (e.g., orally and/or oro/naso-gastric), F being from about 0.7 to about 3.3 grams (per dose), D being about 80 to about 160 mg (per dose), and S being about 360 to about 800 mg (per dose). A plurality of active co-agents may comprise at least an F, a D, and an S (each singly active against an aerobic bacteria in an aerobic environ, and at least one being singly inactive against an anaerobe in an anaerobic environ), are co-dosed daily every eight hours or every twelve hours (e.g., orally and/or oro/naso-gastric), F being from about 1.4 to about 6.7 grams (per dose), D being about 160 to about 320 mg (per dose), and S being about 700 to about 1800 mg (per dose). A plurality of active co-agents may comprise at least an F, a D, and an S, are co-dosed daily every six hours (e.g., enema or retentive enema), F being from about 500 to about 4 grams (per dose), D being about 80 to about 640 mg (per dose), and S being about 360 mg to about 3.2 g (per dose). D and S may be provided as a duo (e.g., D is trimethoprim and S is sulfamethoxazole). One or more of FMEI, PGSI and/or BLI herein may be co-dosed with F, D, S. In some embodiments, PGSI and BLI may be provided as a duo or a trio (e.g., when indicated and/or presented/prepared as a duo or trio for approved use).

Representative delivery or dispensing examples are provided, in which a daily co-dosing therapy against one or more pathogenic anaerobes (which is or includes an obligate anaerobe in an anaerobic environment) is or includes at least F, D, S or F, Q, S, and continues until an infection and/or symptoms improve, are controlled or resolved, or for a safety concern. For parenteral delivery or dispensing, F may be provided in dry/powder or liquid form in a vial, and is or may be fosfomycin tromethamine salt or fosfomycin disodium (or hydrolysable) salt (as representative examples for purposes herein only). D may be provided in dry/powder or liquid form in a vial, and is or may be trimethoprim, iclaprim, pyrimethamine (as representative examples for purposes herein only). S may be provided in dry/powder or liquid form in a vial, and is or may be sulfamethoxazole, sulfisoxazole, sulfadoxin (as representative examples for purposes herein only). D may be substituted by Q, which is or may be SCH 79797, irresistin, gefitinib, lapatinib, erlotinib, afatinib (as representative examples for purposes herein only) provided in dry/powder or liquid form in a vial or as tablet. Forms in a vial are dissolved and/or reconstituted and/or diluted (e.g., with 5% dextrose, buffered saline, sterile water or other suitable diluent) for injection/IV. Vials may be provided in a kit with or without one or more diluents. Two or more co-agents may be in a same vial. Daily co-dosing via an IV route utilizes a peripheral and/or central line. Co-dosing may be in a same or different IV lines (e.g., different when forms are not compatible), and comprises at least F at about 1.8 to 7.5 g every 6 hours (4 times/day) and D at about 5 to 20 mg/kg every 6 hours (4 times/day) and S at about 25-100 mg/kg every 6 hours (4 times/day), in which IV infusion is metered (e.g., via pump) for delivery or dispensing over a period (e.g., up to or about 60 min or up to or about 90 min or up to or about 120 min). When co-dosing further comprises FMEI, PGSI and/or BLI, the FMEI, PGSI and/or BLI will be on a similar schedule (e.g., every six hours at a same or substantially same period as F, D, S, or F, Q, S).

For oral or oro/naso-gastric delivery or dispensing, F may be provided in dry/powder or liquid or solid form (dry/powder may be in a sachet or vial, solid as a tablet or capsule or some equivalent), and is or may be fosfomycin tromethamine salt or fosfomycin disodium salt (exemplified for purposes herein only). D may be provided in dry/powder or liquid or solid form (dry/powder may be in a sachet or vial, solid as a tablet or capsule or some equivalent), and is or may be trimethoprim, iclaprim, pyrimethamine (exemplified for purposes herein only). S may be provided in dry/powder or liquid or solid form (dry/powder may be in a sachet or vial, solid as a tablet or capsule or some equivalent), and is or may be sulfamethoxazole, sulfisoxazole, sulfadoxin (as representative examples for purposes herein only). D may be substituted by Q, which is or may be SCH 79797, irresistin, gefitinib, lapatinib, erlotinib, afatinib (exemplified for purposes herein only) provided, e.g., in dry/powder or liquid form in a vial, sachet, or as tablet or capsule. Forms in a vial or sachet, e.g., in dry/powder form, are dissolved and/or reconstituted and/or diluted (e.g., with 5% dextrose, buffered saline, sterile water, or other diluent) prior to delivery or dispensing via an oral route or an oro- or naso-gastric tube. Vials and/or solids and/or sachets may be in a kit with or without one or more diluents. Two or more co-agents may be in a same vehicle (e.g., vial or sachet or pill or capsule or elixir or concentrate, as examples). Co-dosing may be in a same solid or liquid or different solids or liquids (e.g., when forms are not compatible), and in a first exemplary embodiment comprises at least F at about 0.7 to 3.4 g every 6 hours (4 times/day) and D at about 80 to 160 mg every 6 hours (4 times/day) and S at about 360 to 800 mg every 6 hours (4 times/day), in which delivery or dispensing is in a same or substantially same period (e.g., up to or about 15 min or up to or about 30 min or up to or about 45 min). In another example, co-dosing comprises at least F at about 1.4 to 6.7 g every 12 hours (2 times/day) and D at about 160 to 320 mg every 12 hours (2 times/day) and S at about 700 to 1200 mg every 12 hours (2 times/day), in which delivery or dispensing is in a same or substantially same period (e.g., up to or about 15 min or up to or about 30 min or up to or about 45 min). When co-dosing further comprises FMEI, PGSI and/or BLI, the FMEI, PGSI and/or BLI will be on a similar schedule (e.g., every six hours or every twelve hours, at a same or substantially same period as F, D, S, or F, Q, S).

For enema or retentive enema delivery or dispensing, F may be provided in dry/powder or liquid form (dry/powder may be in a sachet or vial), and is or may be fosfomycin tromethamine salt or fosfomycin disodium (or hydrolyzable) salt (representative examples for purposes herein only). D may be provided in dry/powder or liquid form (dry/powder may be in a sachet or vial), and is or may be trimethoprim, iclaprim, pyrimethamine (representative examples for purposes herein only). S may be provided in dry/powder or liquid form (dry/powder may be in a sachet or vial), and is or may be sulfamethoxazole, sulfisoxazole, sulfadoxin (as representative examples for purposes herein only). D may be substituted by Q, which is or may be SCH 79797, irresistin, gefitinib, lapatinib, erlotinib, afatinib (representative examples for purposes herein only) provided, e.g., in dry/powder or liquid form. Forms in a vial or sachet, e.g., in dry/powder form, are dissolved and/or reconstituted and/or diluted (e.g., with 5% dextrose, buffered saline, sterile water, or other appropriate diluent). Co-agents in a same or different volume are diluted to a fixed volume (e.g., up to or about 100 ml, up to or about 250 ml, up to or about 500 ml). Vials and/or sachets may be provided in a kit with or without one or more diluents, with or without a bag or balloon and/or catheter and/or inflation means. Two or more co-agents may be in a same vehicle (e.g., vial or sachet or elixir or concentrate, as examples). Co-dosing may be in a same bag or balloon, or different bags or balloons (e.g., when forms are not compatible), involving a catheter with a bag or balloon inserted in the rectum, inflated, and a fixed volume comprising one or more co-agents is instilled, followed by clamping of the catheter for a period (e.g., up to or about 60 min or up to or about 90 min or up to or about 120 min), in which F is about 500 mg to 4 g and D is about 80 to 640 mg and S is about 360 mg to 3.2 g, each provided every 6 hours (4 times/day). When utilizing different bags or balloons, delivery or dispensing is in series. Co-dosing may further comprise FMEI, PGSI and/or BLI, in which FMEI, PGSI and/or BLI will be in a same or subsequent bag or balloon as one or more of F, D, S (or F, Q, S).

In some embodiments, is a kit comprising one or a plurality of vehicles (e.g., vial, sachet, pill, tablet, suppository, as representative examples only), in which at least one pharmaceutically acceptable co-agent (pre-weighed, pre-measured for at least one dosage per vehicle, generally with one or more excipients, with one or more days of dosing) is in the one or the plurality of vehicles in the kit. In one or more embodiments, is a kit comprising separate vehicles for each co-agent (e.g., one for F, one for D (or Q), one for S, etc.), in which each co-agent (pre-weighed, pre-measured for at least one dosage and/or for one day of dosing, generally with one or more excipients) is in its vehicle in the kit. In one or more embodiments, is a kit comprising a same vehicle for the plurality of co-agents, in which the plurality of co-agent (pre-weighed, pre-measured for at least one dosage per vehicle, generally with one or more excipients, with one or more days of dosing) are in a same vehicle in the kit. In one or more embodiments, a kit further comprises information/instructions. In one or more embodiments, a kit further comprises a suitable diluent for the one or plurality of vehicles.

The inventors have demonstrated that antimicrobials herein (each comprising a plurality of co-agents being co-dosed in a period) are active against target anaerobes in an anaerobic environment, including difficult to treat anaerobic pathogens that may present in a mixed infection, that cannot utilize oxygen, and/or that are inhibited by oxygen (i.e., obligate anaerobes). It was also shown that said antimicrobials increase activity or efficiency of a co-agent when combined herein, in a synergistic manner. Further, an alternate inhibitor of bacterial dihydrofolate reductase may be successfully substituted for a diaminopyrimidine to achieve equally effective synergistic activity against such anaerobes. Unexpectedly, it was demonstrated that inventive antimicrobials herein are selective in activity. While able to target and be active against all obligate anaerobes tested, the co-agent compositions and/or formulations herein were inactive against what are considered mutualistic or beneficial commensal bacteria of a normal GI system, or the facultative anaerobes Lactobacillus spp., Pediococcus spp. and/or Leuconostoc, which were not inhibited by nor found to be responsive (nor susceptible) to inventive antimicrobial herein. Such unique and unexpected findings herein show that antimicrobials herein provide a means for targeted pharmacotherapy against select pathogenic anaerobes, and provide a new and novel approach to direct therapy specifically against anaerobic pathogenic organisms. The importance of such findings means a reduction in clinical failures, because (according to reports by other skilled artisans in the field) failure to direct therapy against anaerobes or use of inappropriate therapy against anaerobes can and often does result in clinical failure. In addition, knowing that use of many alternative approved antibiotics are associated with an adverse phenomenon known as dysbiosis (GI system side effects and an increase in infection in the GI system, including increased risk of infection from one or more obligate anaerobes, such as C. difficile, which is due to death of protective GI microflora or those considered mutualistic and/or beneficial), the findings herein regarding selective inactivity against at least some mutualistic or beneficial commensal bacteria of a normal GI system, mean utilization of at least some inventive antimicrobials herein should result in an overall reduction in GI side effects and GI infections, and will be suitable for use for a host (e.g., patient) with a pre-existing GI issue or prone to recurrent GI infection.

Based on in vitro activity of the many active co-agent pluralities tested, it is clear that providing (by a co-dosing in a period) any of said active co-agents herein in a combination and/or formulation, and utilizing any such combination and/or formulation against one or more pathogenic anaerobes in a host or subject that is known or suspected of having said one or more anaerobes, will provide a truly effective antimicrobial, as well as a good clinical outcome, thereby improving treatment time and reducing mortality rates. In addition, based on in vitro activity of the copious active co-agent pluralities tested, it is clear that active co-agents herein in a combination and/or formulation are as effective or more effective than metronidazole, which has become unreliable against Gram-positive anaerobes, particularly GPOAs, as reported by others of skill in the field. Based on in vitro activity of the copious active co-agent pluralities tested, it is clear that active co-agents herein in a combination and/or formulation are more effective than clindamycin against GNOAs, since, overall, clindamycin is found by others of skill in the field to be less reliable than metronidazole, and continues to show increasing resistance to certain obligate anaerobes, including Bacteroides, moreover clindamycin does not penetrate the central nervous system, while active co-agent pluralities herein (e.g., at least F, D, S, or F, Q, S) will penetrate the blood-brain barrier and enter the CNS. Based on in vitro activity of the copious active co-agent pluralities tested, it is clear that active co-agents herein in a combination and/or formulation are more effective than penicillins, which, as reported by others of skill in the field, have become unreliable due to an ever increasing resistance profile and to their inactivity against anaerobes that produce beta-lactamase (which describes most Gram-negative anaerobes), while co-agents herein are active against beta-lactamase producing anaerobes. Based on in vitro activity of the copious active co-agent pluralities tested, it is clear that active co-agents herein in a combination and/or formulation are more effective than second-generation cephalosporins (e.g., cefoxitin, cefotetan, cefmetazole), which, as reported by others of skill in the field, have become unreliable and are not recommended as empiric treatment due to their increasing resistance profile. Based on in vitro activity of the copious active co-agent pluralities tested, it is clear that active co-agents herein in a combination and/or formulation are more effective than fluoroquinolones (e.g., levofloxacin, moxifloxacin), which, as reported by others of skill in the field, have become unreliable and are generally reserved as salvage therapy after failure of other agents or for those with beta-lactam allergy due to their increasing resistance profile.

As used herein, and in the appended claims, the singular forms “a,” “and,” and “the” may include plural referents unless context clearly dictates otherwise. For example, “a combination” or “a formulation” may include a plurality, and equivalents thereof as is known to those skilled in the art. It is understood that the term “comprising” (and related terms such as “comprise” or “comprises” or “having” or “including”) is not intended to exclude embodiments wherein, for example, a combination, composition, method, or process, or the like, described herein, may “consist of” or “consist essentially of” the certain described features.

TABLE 1
Synergy of antimicrobial combination (fosfomycin (F) & diaminopyridine
(D) & sulfonamide (S)) against obligate anaerobic pathogens.

| | MIC (μg/ml) | | | | |
|---|---|---|---|---|---|
| | F | D/S | F/D/S | FIC | Interpretation |
| Gram-positive | | | | | |
| Clostridium perfringens 12-117 | 32 | >32/>608 | 8/1/19 | 0.27 | S |
| Clostridium perfringens 11-46 | 16 | >32/>608 | 1/4/76 | 0.12 | S |
| Clostridium perfringens 11-50 | 16 | >32/>608 | 1/8/152 | 0.19 | S |
| Clostridium perfringens 11-61 | 8 | >32/>608 | 1/8/152 | 0.25 | S |
| Clostridium perfringens 2-0246-4 | 16 | >32/>608 | 2/0.06/1.19 | 0.06 | S |
| Clostridioides difficile 3/8 | 8 | 8/152 | 0.25/2/38 | 0.28 | S |
| Clostridioides difficile 2-0247-4 | 16 | 8/152 | 0.25/2/38 | 0.27 | S |
| Clostridioides difficile 10-3620-5 | 8 | 8/152 | 0.25/2/38 | 0.28 | S |
| Clostridium septicum 11-3571 | 16 | >32/>608 | 2/0.5/9.5 | ≤0.13 | S |
| Clostridium septicum 15-847 | 32 | 8/152 | 2/2/38 | ≤0.31 | S |
| Bifidobacterium animalis JLP-002 | 128 | 0.5/9.5 | 0.125/≤0.03/≤0.6 | ≤0.07 | S |
| Peptostreptococcus anaerobious 807256 | 32 | 4/76 | 8/1/19 | 0.5 | S |
| Cutibacterium acnes 6609 | 32 | 32/608 | 0.25/1/19 | 0.04 | S |
| Streptococcus mutans UA 159 | 16 | >32/>608 | 4/2/38 | 0.28 | S |
| Gram-negative | | | | | |
| Bacteroides fragilis ATCC 23745 | >128 | 8/152 | 0.125/2/38 | 0.25 | S |
| Bacteroides fragilis ATCC 25285 | >128 | 8/152 | 0.125/2/38 | 0.25 | S |
| Bacteroides fragilis CLA267 | >128 | 8/182 | 0.125/2/38 | 0.25 | S |
| Bacteroides fragilis O16M | >128 | 8/152 | 0.125/2/38 | 0.25 | S |
| Bacteroides fragilis 638R | >128 | 8/152 | 0.125/2/38 | 0.25 | S |
| Bacteroides ovatus ATCC 8483 | >128 | 4/76 | 0.25/1/19 | 0.25 | S |
| Bacteroides thetaiotaomicron VPI5482 | >128 | 4/76 | 0.125/1/19 | 0.25 | S |
| Phocaeicola vulgatus ATCC 8482 | >128 | 8/152 | 0.25/2/38 | 0.25 | S |
| Phocaeicola vulgatus ATCC 29327 | >128 | 8/152 | 0.5/2/38 | 0.25 | S |
| Parabacteroides distasonis CLA348 | >128 | >32/>608 | 1/4/76 | 0.07 | S |
| Prevotella intermedia 807114 | >128 | 32/608 | 1/0.5/9.5 | 0.02 | S |
| Porphyromonas gingivalis ATCC 33277 | >128 | 0.25/4.75 | ≤1/≤0.015/≤0.3 | ≤0.03 | S |
| Aggretibacter actinomycetemcomitans D751 | 16 | 4/76 | 0.25/1/19 | 0.27 | S |

D = trimethoprim (a diaminopyridine); S = sulfamethoxazole (a sulfonamide).

TABLE 2
Synergy of antimicrobial combination (F, D, S) against obligate anaerobic pathogens.

| | MIC (μg/ml) | | | | |
|---|---|---|---|---|---|
| | F | D/S | F/D/S | FIC | Interpretation |
| Gram-positive | | | | | |
| Clostridium perfringens 12-117 | 32 | 32/608 | 2/4/76 | 0.19 | S |
| Clostridium perfringens 11-46 | 32 | >32/>608 | 2/8/152 | 0.19 | S |
| Clostridium perfringens 11-50 | 16 | 32/608 | 2/8/152 | 0.38 | S |
| Clostridium perfringens 11-61 | 16 | 8/152 | 4/1/19 | 0.38 | S |
| Clostridium perfringens 2-0246-4 | 16 | 2/38 | 4/0.5/9.5 | 0.5 | S |
| Clostridioides difficile 3/8 | 16 | 32/608 | 4/1/19 | 0.28 | S |
| Clostridioides difficile 2-0247-4 | 16 | 2/38 | 4/0.5/9.5 | 0.5 | S |
| Clostridioides difficile 10-3620-5 | 8 | 8/152 | 1/1/19 | 0.25 | S |
| Bifidobacterium animalis JLP-002 | 128 | 2/38 | 8/0.5/0.5 | 0.5 | S |
| Peptostreptococcus anaerobious 807256 | 32 | 0.25/4.75 | ≤1/0.06/1.19 | ≤0.28 | S |
| Cutibacterium acnes 6609 | 64 | 0.125/2.38 | ≤1/0.03/0.6 | ≤0.27 | S |
| Gram-negative | | | | | |
| Bacteroides fragilis ATCC 25285 | >128 | 1/19 | 32/0.25/4.75 | 0.38 | S |
| Bacteroides fragilis ATCC 23745 | >128 | 1/19 | 32/0.25/4.75 | 0.38 | S |
| Bacteroides fragilis O16M | >128 | 2/38 | 32/0.25/4.75 | 0.38 | S |
| Bacteroides fragilis CLA267 | >128 | 2/38 | 32/0.25/4.75 | 0.38 | S |
| Bacteroides fragilis 638R | >128 | 2/38 | 32/0.5/9.5 | 0.38 | S |
| Bacteroides ovatus ATCC 8483 | >128 | 2/38 | 32/0.5/9.5 | 0.38 | S |
| Bacteroides thetaiotaomicron VPI5482 | >128 | 1/19 | 32/0.25/4.75 | 0.38 | S |
| Phocaeicola vulgatus ATCC 29327 | >128 | 2/38 | 32/0.5/9.5 | 0.38 | S |
| Phocaeicola vulgatus ATCC 8482 | >128 | 4/76 | 32/0.5/9.5 | 0.25 | S |
| Parabacteroides distasonis CLA348 | >128 | 2/38 | 32/0.5/9.5 | 0.38 | S |
| Prevotella intermedia 807114 | >128 | 0.125/4.75 | 1/0.015/0.3 | 0.13 | S |

D = trimethoprim (a diaminopyridine); S = sulfisoxazole (a sulfonamide).

TABLE 3
Synergy of antimicrobial combination (F, D, S) against obligate anaerobic pathogens.

| | MIC (μg/ml) | | | | |
|---|---|---|---|---|---|
| | F | D/S | F/D/S | FIC | Interpretation |
| Gram-positive | | | | | |
| Clostridium perfringens 12-117 | 32 | >32/608 | 8/8/152 | ≤0.38 | S |
| Clostridium perfringens 11-46 | 16 | >32/608 | 4/4/76 | ≤0.31 | S |
| Clostridium perfringens 11-50 | 16 | >32/608 | 4/4/76 | ≤0.31 | S |
| Clostridium perfringens 11-61 | 8 | >32/608 | 2/4/76 | ≤0.31 | S |
| Clostridium perfringens 2-0246-4 | 16 | 16/304 | 4/4/76 | 0.5 | S |
| Clostridioides difficile 3/8 | 8 | 4/76 | 2/1/19 | 0.5 | S |
| Clostridioides difficile 2-0247-4 | 16 | >32/>608 | 4/8/152 | ≤0.38 | S |
| Clostridioides difficile 10-3620-5 | 8 | >32/>608 | 1/4/76 | ≤0.19 | S |
| Bifidobacterium animalis JLP0002 | 128 | 0.125/2.38 | 2/0.03/0.6 | 0.27 | S |
| Peptostreptococcus anaerobius 807256 | 32 | 4/76 | 8/1/19 | 0.5 | S |
| Cutibacterium acnes 6609 | 32 | >32/>608 | 8/2/38 | ≤0.28 | S |
| Gram-negative | | | | | |
| Bacteroides fragilis ATCC 23745 | >128 | 32/608 | 32/8/152 | ≤0.38 | S |
| Bacteroides fragilis ATCC 25285 | >128 | >32/>608 | 32/8/152 | ≤0.25 | S |
| Bacteroides fragilis 016M | >128 | 8/152 | 2/2/38 | ≤0.26 | S |
| Bacteroides fragilis CLA 267 | >128 | 8/152 | 2/2/38 | ≤0.26 | S |
| Bacteroides fragilis 638R | >128 | 8/152 | 2/2/38 | ≤0.26 | S |
| Bacteroides ovatus ATCC 8483 | >128 | 8/152 | 2/2/38 | ≤0.26 | S |
| Bacteroides thetaiotaomicron VPI 5842 | >128 | 4/76 | 32/1/19 | ≤0.38 | S |
| Phocaeicola vulgatus ATCC 29327 | >128 | 4/76 | 32/1/19 | ≤0.38 | S |
| Phocaeicola vulgatus ATCC 8482 | >128 | >32/>608 | 1/4/76 | ≤0.07 | S |
| Parabacteroides distasonis CLA 348 | >128 | 16/304 | 8/4/76 | ≤0.28 | S |
| Prevotella intermedia 807114 | >128 | 4/76 | 16/1/19 | ≤0.31 | S |

D = trimethoprim (a diaminopyridine); S = sulfadoxine (a sulfonamide).

TABLE 4
Syner [symbol] of antimicrobial co [symbol] bination (F, D, S) ag [symbol] inst obligate anaero [symbol] ic pathogens.

| | MIC (μg/m [symbol] | | | | |
|---|---|---|---|---|---|
| | F | D/S | F/D/S | FIC | Interpretation |
| [symbol] ram-positive | | | | | |
| Clostridium per [symbol] ingens 12-117 | 16 | >32/>608 | 4/1/19 | ≤0.27 | S |
| Clostridium per [symbol] ingens 11-46 | 16 | >32/>608 | 4/8/152 | ≤0.38 | S |
| Clostridium per [symbol] ingens 11-50 | 16 | 16/304 | 4/0.5/9.5 | 0.28 | S |
| Clostridium per [symbol] ingens 11-61 | 16 | 16/304 | 4/1/19 | 0.31 | S |
| Clostridium per [symbol] ingens 2-0246-4 | 16 | 8/152 | 4/2/38 | 0.5 | S |
| Clostridioides d [symbol] ficile 3/8 | 16 | 32/608 | 4/2/38 | 0.33 | S |
| Clostridioides d [symbol] ficile 10-3620-5 | 16 | >32/>608 | 4/2/38 | ≤0.28 | S |
| Clostridioides d [symbol] ficile 2-0247-4 | 16 | 1/19 | 4/0.25/4.75 | 0.5 | S |
| Cutibacterium [symbol] nes 6609 | 64 | 2/38 | 4/0.25/4.75 | 0.19 | S |
| Peptostreptoco [symbol] us anaerobious 807 [symbol] 56 | 32 | 16/304 | 4/0.25/4.75 | 0.13 | S |
| Bifidobacterium animalis TBC0002 | 128 | 8/152 | 1/1/19 | 0.14 | S |
| [symbol] ram-negative | | | | | |
| Bacteroides fra [symbol] lis ATCC 25285 | >128 | >32/>608 | 2/1/19 | ≤0.02 | S |
| Bacteroides fra [symbol] lis ATCC 23745 | >128 | >32/>608 | 16/2/38 | ≤0.31 | S |
| Bacteroides fra [symbol] lis CLA 267 | >128 | 32/608 | 4/0.5/9.5 | ≤0.14 | S |
| Bacteroides fra [symbol] lis 016M | >128 | 2/38 | 4/0.5/9.5 | ≤0.27 | S |
| Bacteroides fra [symbol] lis 638R | >128 | 2/38 | 2/0.5/9.5 | ≤0.26 | S |
| Bacteroides ov [symbol] us ATCC 8483 | >128 | 1/19 | 32/0.25/4.75 | ≤0.38 | S |
| Bacteroides the [symbol] aiotaomicron VPI 58 [symbol] 2 | >128 | 1/19 | 8/0.25/4.75 | ≤0.28 | S |
| Phocaeicola vul [symbol] atus ATCC 8482 | >128 | 1/19 | 8/0.25/4.75 | ≤0.28 | S |
| Phocaeicola vul [symbol] atus ATCC 29327 | >128 | 1/19 | 16/0.25/4.75 | ≤0.31 | S |
| Prevotella inter [symbol] edia 8071621 | 64 | 1/19 | 8/0.25/4.75 | 0.5 | S |
| Parabacteroide [symbol] distasonis CLA 348 | >128 | 4/76 | 4/0.25/4.75 | 0.08 | S |

D = pyrimethamin [symbol] (a diaminopyridine); [symbol] = sulfamethoxazole (a sulfonamide). [symbol]
indicates data missing or illegible when filed

[symbol]

ABLE 5
Synergy of a [symbol] imicrobial combina [symbol] on (F, D, S) against [symbol] ligate anaerobic pa [symbol] hogens.

| | MIC (μg/ml) | | | | |
|---|---|---|---|---|---|
| | F | D/S | F/D/S | FIC | Interpretation |
| Gra [symbol] -positive | | | | | |
| Clostridium perfring [symbol] ns 12-117 | 32 | >32/>608 | 8/1/19 | ≤0.27 | S |
| Clostridium perfring [symbol] ns 11-46 | 32 | 32/608 | 8/1/19 | 0.28 | S |
| Clostridium perfring [symbol] ns 11-50 | 16 | 4/76 | 1/1/19 | 0.31 | S |
| Clostridium perfring [symbol] ns 11-61 | 32 | 2/38 | 1/0.5/9.5 | 0.28 | S |
| Clostridioides diffic [symbol] 3/8 | 16 | 16/304 | 1/1/19 | 0.13 | S |
| Clostridioides diffic [symbol] 2-0247-4 | 16 | 2/38 | 4/0.5/9.5 | 0.5 | S |
| Cutibacterium acne [symbol] 6609 | 64 | 1/19 | 1/0.06/1.19 | 0.08 | S |
| Bifidobacterium ani [symbol] alis TBC0002 | 128 | 1/19 | 1/0.25/4.75 | 0.27 | S |
| Peptostreptococcus [symbol] naerobious 807256 | 32 | 2/38 | 8/0.015/0.3 | 0.26 | S |
| Gra [symbol] -negative | | | | | |
| Bacteroides fragilis [symbol] TCC 25285 | 128 | 1/19 | 4/0.25/4.75 | 0.28 | S |
| Bacteroides fragilis [symbol] TCC 23745 | >128 | 1/19 | 4/0.25/4.75 | ≤0.27 | S |
| Bacteroides fragilis [symbol] LA 267 | >128 | 1/19 | 8/0.25/4.75 | ≤0.28 | S |
| Bacteroides fragilis [symbol] 16M | >128 | 0.5/9.5 | 16/0.125/2.38 | ≤0.31 | S |
| Bacteroides fragilis [symbol] 8R | >128 | 0.5/9.5 | 1/0.125/2.38 | ≤0.25 | S |
| Bacteroides ovatus [symbol] TCC 8483 | >128 | 0.5/9.5 | 2/0.125/2.38 | ≤0.26 | S |
| Bacteroides thetaio [symbol] omicron VPI 5842 | >128 | 0.5/9.5 | 1/0.125/2.38 | ≤0.25 | S |
| Phocaeicola vulgat [symbol] ATCC 29327 | >128 | 0.5/9.5 | 1/0.125/2.38 | ≤0.25 | S |
| Phocaeicola vulgat [symbol] ATCC 8482 | >128 | 1/19 | 2/0.25/4.75 | ≤0.26 | S |
| Prevotella intermed [symbol] 807114 | >128 | 0.06/1.19 | 1/≤0.015/≤0.3 | ≤0.26 | S |
| Parabacteroides dis [symbol] asonis CLA348 | >128 | 4/76 | 8/0.5/9.5 | ≤0.16 | S |

[symbol]

= pyrimethamine (a [symbol] aminopyridine); S = su [symbol] soxazole (a sulfona [symbol] de). [symbol]
indicates data missing or illegible when filed

TABLE 6
Synergy [symbol] f antimicrobial co [symbol] ination (F, D, S) aga [symbol] st obligate anaerob [symbol] pathogens.

| | MIC (μg/m [symbol] | | | | |
|---|---|---|---|---|---|
| | [symbol] | D/S | F/D/S | FIC | Interpretation |
| [symbol] ram-positive | | | | | |
| Clostridium per [symbol] ingens 12-117 | 16 | >32/>608 | 4/8/152 | ≤0.38 | S |
| Clostridium per [symbol] ingens 11-46 | 64 | >32/>608 | 4/8/152 | ≤0.19 | S |
| Clostridium per [symbol] ingens 11-50 | 8 | >32/>608 | 2/2/38 | ≤0.28 | S |
| Clostridium per [symbol] ingens 11-61 | 16 | >32/>608 | 4/2/38 | ≤0.28 | S |
| Clostridium per [symbol] ingens 2-0246-4 | 16 | >32/>608 | 1/0.25/4.75 | ≤0.07 | S |
| Clostridioides d [symbol] icile 3/8 | 16 | 16/304 | 1/0.25/4.75 | 0.08 | S |
| Clostridioides d [symbol] icile 2-0247-4 | 16 | 16/304 | 1/0.5/9.5 | 0.09 | S |
| Clostridioides d [symbol] icile 10-3620-5 | 16 | >32/>608 | 1/1/19 | ≤0.08 | S |
| Bifidobacterium animalis TBC0002 | 128 | 2/38 | 1/0.25/4.75 | 0.16 | S |
| Peptostreptoco [symbol] us anaerobius 807 [symbol] 6 | 16 | >32/>608 | 1/0.25/4.75 | ≤0.07 | S |
| Cutibacterium [symbol] nes 6609 | 64 | >32/>608 | 1/0.25/4.75 | ≤0.02 | S |
| [symbol] ram-negative | | | | | |
| Bacteroides fra [symbol] lis ATCC 25285 | >128 | 16/304 | 1/0.5/9.5 | ≤0.04 | S |
| Bacteroides fra [symbol] lis ATCC 23745 | >128 | 16/304 | ≤1/≤0.06/≤1. [symbol] | ≤0.04 | S |
| Bacteroides fra [symbol] lis CLA267 | >128 | 16/304 | ≤1/≤0.06/≤1. [symbol] | ≤0.01 | S |
| Bacteroides fra [symbol] lis 638R | >128 | 32/608 | ≤1/≤0.06/≤1. [symbol] | ≤0.01 | S |
| Bacteroides fra [symbol] lis 016M | >128 | 32/608 | 1/0.5/9.5 | ≤0.02 | S |
| Bacteroides ov [symbol] us ATCC 8483 | >128 | 4/76 | ≤1/≤0.06/≤1. [symbol] | ≤0.05 | S |
| Bacteroides the [symbol] iotaomicron VPI 58 [symbol] 2 | >128 | >32/>608 | 1/0.25/4.75 | ≤0.01 | S |
| Phocaeicola vul [symbol] atus ATCC 29327 | >128 | 4/76 | ≤1/≤0.06/≤1. [symbol] | ≤0.02 | S |
| Phocaeicola vul [symbol] atus ATCC 8482 | >128 | 16/304 | 1/0.25/4.75 | ≤0.02 | S |
| Prevotella inter [symbol] edia 807114 | >128 | 32/608 | 1/0.25/4.75 | ≤0.01 | S |
| Parabacteroide [symbol] distasonis CLA 348 | >128 | 32/608 | 1/0.25/4.75 | ≤0.01 | S |

D = pyrimethamin [symbol] (a diaminopyridine); [symbol] = sulfadoxine (a sulfo [symbol] mide). [symbol]
indicates data missing or illegible when filed

[symbol]

ABLE 7
Synergy of a [symbol] timicrobial combina [symbol] on (F, D, S) against [symbol] ligate anaerobic pa [symbol] ogens.

| | MIC (μg/ml) | | | | |
|---|---|---|---|---|---|
| | F | D/S | F/D/S | FIC | Interpretation |
| Gra [symbol] -positive | | | | | |
| Clostridium perfring [symbol] ns 12-117 | 32 | >32/>608 | 8/1/19 | ≤0.27 | S |
| Clostridium perfring [symbol] ns 11-46 | 32 | 32/608 | 2/1/19 | 0.09 | S |
| Clostridium perfring [symbol] ns 11-50 | 16 | 4/76 | 1/1/19 | 0.31 | S |
| Clostridium perfring [symbol] ns 11-61 | 32 | 2/38 | 1/0.5/9.5 | 0.28 | S |
| Clostridioides diffici [symbol] 3/8 | 16 | 16/304 | 1/1/19 | 0.13 | S |
| Clostridioides diffici [symbol] 2-0247-4 | 16 | 2/38 | 4/0.5/9.5 | 0.5 | S |
| Cutibacterium acne [symbol] 6609 | 64 | 1/19 | 1/0.06/1.19 | 0.08 | S |
| Bifidobacterium an [symbol] alis TBC0002 | 128 | 1/19 | 1/0.25/4.75 | 0.27 | S |
| Peptostreptococcus [symbol] naerobious 807256 | 32 | 2/38 | 8/0.015/0.3 | 0.26 | S |
| Gra [symbol] -negative | | | | | |
| Bacteroides fragilis [symbol] TCC 25285 | 128 | 1/19 | 4/0.25/4.75 | 0.28 | S |
| Bacteroides fragilis [symbol] TCC 23745 | >128 | 1/19 | 4/0.25/4.75 | ≤0.27 | S |
| Bacteroides fragilis [symbol] LA 267 | >128 | 1/19 | 8/0.25/4.75 | ≤0.28 | S |
| Bacteroides fragilis [symbol] 16M | >128 | 0.5/9.5 | 16/0.125/2.38 | ≤0.31 | S |
| Bacteroides fragilis [symbol] 38R | >128 | 0.5/9.5 | 1/0.125/4.75 | ≤0.25 | S |
| Bacteroides ovatus [symbol] TCC 8483 | >128 | 0.5/9.5 | 2/0.125/4.75 | ≤0.27 | S |
| Bacteroides thetaia [symbol] omicron VPI 5842 | >128 | 0.5/9.5 | 1/0.125/2.38 | ≤0.25 | S |
| Phocaeicola vulgat [symbol] ATCC 29327 | >128 | 0.5/9.5 | 1/0.125/2.38 | ≤0.25 | S |
| Phocaeicola vulgat [symbol] ATCC 8482 | >128 | 1/19 | 2/0.25/9.5 | ≤0.26 | S |
| Parabacteroides dis [symbol] sonis CLA 348 | >128 | 1/19 | 32/0.25/4.75 | ≤0.38 | S |
| Prevotella intermed [symbol] 8071621 | >128 | 0.06/1.19 | 1/≤0.015/≤0.3 | ≤0.25 | S |

[symbol]

= iclaprim (a diamino [symbol] yridine); S = sulfamet [symbol] xazole (a sulfonami [symbol] ). [symbol]
indicates data missing or illegible when filed

TABLE 8
Synergy [symbol] f antimicrobial com [symbol] ination (F, D, S) aga [symbol] st obligate anaerob [symbol] pathogens.

| | MIC (μg/m [symbol] | | | | |
|---|---|---|---|---|---|
| | F | D/S | F/D/S | FIC | Interpretation |
| [symbol] ram-positive | | | | | |
| Clostridium per [symbol] ingens 12-117 | 32 | >32/>608 | 8/1/19 | ≤0.27 | S |
| Clostridium per [symbol] ingens 11-46 | 32 | 32/608 | 8/1/19 | 0.28 | S |
| Clostridium per [symbol] ingens 11-50 | 16 | 4/76 | 1/1/19 | 0.31 | S |
| Clostridium per [symbol] ingens 11-61 | 32 | 2/38 | 1/0.5/9.5 | 0.28 | S |
| Clostridioides d [symbol] ficile 3/8 | 16 | 16/304 | 1/1/19 | 0.13 | S |
| Clostridioides d [symbol] ficile 2-0247-4 | 16 | 2/38 | 4/0.5/9.5 | 0.5 | S |
| Cutibacterium [symbol] nes 6609 | 128 | 1/19 | 1/0.06/1.19 | 0.07 | S |
| Bifidobacterium animalis TBC0002 | 128 | 1/19 | 1/0.25/4.75 | 0.27 | S |
| Peptostreptoco [symbol] us anaerobious 807 [symbol] 56 | 32 | 2/38 | 8/0.015/0.3 | 0.26 | S |
| [symbol] ram-negative | | | | | |
| Bacteroides fra [symbol] lis ATCC 25285 | 128 | 1/19 | 4/0.25/4.75 | 0.28 | S |
| Bacteroides fra [symbol] lis ATCC 23745 | >128 | 1/19 | 4/0.25/4.75 | ≤0.27 | S |
| Bacteroides fra [symbol] lis CLA267 | >128 | 1/19 | 8/0.25/4.75 | ≤0.28 | S |
| Bacteroides fra [symbol] lis 016M | >128 | 0.5/9.5 | 16/0.125/2.38 | ≤0.31 | S |
| Bacteroides fra [symbol] lis 638R | >128 | 0.5/9.5 | 1/0.125/2.38 | ≤0.25 | S |
| Bacteroides ov [symbol] us ATCC 8483 | >128 | 0.5/9.5 | 1/0.125/2.38 | ≤0.25 | S |
| Bacteroides the [symbol] iotaomicron VPI 58 [symbol] 2 | >128 | 0.5/9.5 | 1/0.125/2.38 | ≤0.25 | S |
| Phocaeicola vul [symbol] atus ATCC 29327 | >128 | 0.8/9.5 | 1/0.125/2.38 | ≤0.25 | S |
| Phocaeicola vul [symbol] atus ATCC 8482 | >128 | 1/19 | 2/0.25/4.75 | ≤0.26 | S |
| Parabacteroide [symbol] distasonis CLA 348 | >128 | 1/19 | 32/0.25/4.75 | ≤0.38 | S |
| Prevotella inter [symbol] edia 807114 | >128 | 0.06/1.19 | 1/≤0.015/≤0.3 | ≤0.25 | S |

D = iclaprim (a dia [symbol] inopyridine); S = sulfis [symbol] xazole (a sulfonamid [symbol] ). [symbol]
indicates data missing or illegible when filed

TABLE 9
Synergy of antimicrobial combination (F, D, S) against obligate anaerobic pathogens.

| | MIC (μg/ml) | | | | |
|---|---|---|---|---|---|
| | F | D/S | F/D/S | FIC | Interpretation |
| Gram-positive | | | | | |
| Clostridium perfringens 12-117 | 32 | >32/>608 | 8/2/38 | ≤0.28 | S |
| Clostridium perfringens 11-46 | 32 | >32/>608 | 8/16/304 | ≤0.5 | S |
| Clostridium perfringens 11-50 | 16 | 32/608 | 4/0.06/1.19 | 0.25 | S |
| Clostridium perfringens 11-61 | 32 | 32/608 | 4/2/38 | 0.19 | S |
| Clostridium perfringens 2-0246-4 | 8 | >32/>608 | 2/0.06/1.19 | ≤0.25 | S |
| Clostridioides difficile 3/8 | 8 | >32/>608 | 2/0.06/1.19 | ≤0.25 | S |
| Clostridioides difficile 2-0247-4 | 16 | >32/>608 | 2/2/38 | ≤0.16 | S |
| Clostridioides difficile 10-3620-5 | 8 | >32/>608 | 2/2/38 | ≤0.28 | S |
| Bifidobacterium animalis TBC0002 | 128 | >32/>608 | 4/1/19 | ≤0.14 | S |
| Peptostreptococcus anaerobius 807256 | 16 | >32/>608 | 2/1/19 | ≤0.14 | S |
| Cutibacterium acnes 6609 | 128 | >32/>608 | 2/0.125/2.38 | ≤0.02 | S |
| Gram-negative | | | | | |
| Bacteroides fragilis ATCC 23745 | >128 | 2/38 | 1/0.06/1.19 | ≤0.03 | S |
| Bacteroides fragilis ATCC 25285 | >128 | 4/76 | 2/0.125/2.38 | ≤0.04 | S |
| Bacteroides fragilis CLA267 | >128 | 2/38 | ≤1/≤0.015/≤0.3 | ≤0.01 | S |
| Bacteroides fragilis 016M | >128 | 4/76 | 1/0.06/1.19 | ≤0.02 | S |
| Bacteroides fragilis 638R | >128 | 4/76 | 1/0.06/1.19 | ≤0.02 | S |
| Bacteroides ovatus ATCC 8483 | >128 | 4/76 | 32/1/19 | 0.38 | S |
| Bacteroides thetaiotaomicron VPI 5842 | >128 | 4/76 | 1/0.06/1.19 | ≤0.02 | S |
| Phocaeicola vulgatus ATCC 8482 | 128 | 4/76 | 1/0.06/1.19 | 0.02 | S |
| Phocaeicola vulgatus ATCC 29327 | 64 | 2/38 | ≤1/≤0.015/≤0.3 | ≤0.02 | S |
| Prevotella intermedia 807114 | >128 | 4/76 | 4/1/19 | ≤0.27 | S |
| Parabacteroides distasonis CLA 348 | >128 | 8/152 | 1/0.06/1.19 | ≤0.01 | S |

D = iclaprim (a diaminopyridine); S = sulfadoxine (a sulfonamide).

TABLE 10A
Synergy of antimicrobial combination (F, quinazoline
(Q), S) against obligate anaerobic pathogens.

| | MIC (μg/ml) | | | | |
|---|---|---|---|---|---|
| | F | Q/S | F/Q/S | FIC | Interpretation |
| Gram-positive | | | | | |
| Clostridium perfringens 12-117 | 32 | 32/608 | 8/4/76 | 0.38 | S |
| Clostridium perfringens 11-46 | 32 | >32/>608 | 2/0.25/4.75 | ≤0.07 | S |
| Clostridium perfringens 11-50 | 8 | 16/304 | 2/2/38 | 0.38 | S |
| Clostridium perfringens 11-61 | 16 | 8/152 | 4/2/38 | 0.5 | S |
| Clostridium perfringens 2-0246-4 | 16 | 16/304 | 4/0.5/9.5 | 0.28 | S |
| Clostridioides difficile 3/8 | 32 | 16/304 | 8/4/76 | 0.5 | S |
| Clostridioides difficile 2-0247-4 | 32 | 32/608 | 4/4/76 | 0.25 | S |
| Clostridioides difficile 10-3620-5 | 8 | 32/608 | 2/0.25/4.75 | 0.26 | S |
| Bifidobacterium animalis TBC0002 | 128 | 16/304 | 16/0.5/9.5 | 0.28 | S |
| Peptostreptococcus anaerobious 807256 | 8 | 16/304 | 2/2/38 | 0.38 | S |
| Cutibacterium acnes 6609 | 128 | 16/304 | 2/2/38 | 0.14 | S |
| Gram-negative | | | | | |
| Bacteroides fragilis ATCC 25285 | >128 | 4/76 | 32/1/19 | ≤0.38 | S |
| Bacteroides fragilis ATCC 23745 | >128 | 0.25/4.75 | ≤1/0.06/1.19 | ≤0.25 | S |
| Bacteroides fragilis 016M | >128 | 8/152 | 16/0.5/0.5 | ≤0.13 | S |
| Bacteroides fragilis 638R | >128 | 0.25/4.75 | 16/0.06/1.19 | ≤0.31 | S |
| Bacteroides fragilis CLA 267 | 128 | 0.25/4.75 | ≤1/0.06/1.19 | ≤0.26 | S |
| Bacteroides ovatus ATCC 8483 | >128 | 16/304 | ≤1/0.25/4.75 | ≤0.02 | S |
| Bacteroides thetaiotaomicron VPI 5842 | >128 | 32/608 | 1/2/38 | ≤0.07 | S |
| Phocaeicola vulgatus ATCC 8482 | >128 | 32/608 | 1/0.5/9.5 | ≤0.02 | S |
| Phocaeicola vulgatus ATCC 29327 | >128 | 8/152 | ≤1/0.25/4.75 | ≤0.04 | S |
| Prevotella intermedia 8071621 | 128 | 0.25/4.75 | ≤1/0.06/1.19 | ≤0.26 | S |
| Parabacteroides distasonis CLA 348 | >128 | 4/76 | 16/0.25/4.75 | ≤0.13 | S |

Q = alternate bacterial DHFR inhibitor, SCH79797; S = sulfamethoxazole (a sulfonamide).

TABLE 10B
Synergy o [symbol] antimicrobial combination (F, Q, S) against facultative anaerobic pathogens.

| | MIC (μg/ml) | | | | |
|---|---|---|---|---|---|
| | F | Q/S | F/Q/S | FIC | Interpretation |
| Gram [symbol] positive | | | | | |
| Staphylococcus aureus (MR) 920516 | 4 | 8/152 | 1/2/38 | 0.5 | S |
| Staphylococcus aureus (MR) 967287 | 8 | >8/>152 | 2/4/76 | ≤0.5 | S |
| Staphylococcus aureus (MR) 956362 | 8 | 8/152 | 1/2/38 | 0.38 | S |
| Staphylococcus aureus (MR) 1022425 | 8 | 8/152 | 1/2/38 | 0.38 | S |
| Staphylococcus aureus (MR) 829025 | 4 | 8/152 | 1/2/38 | 0.5 | S |
| Staphylococcus aureus (MR) 956270 | 4 | 8/152 | 1/2/38 | 0.5 | S |
| Staphylococcus aureus (MR) 979234 | 8 | 8/152 | 1/2/38 | 0.38 | S |
| Staphylococcus aureus (MR) 884649 | 8 | 8/152 | 1/2/38 | 0.38 | S |
| Staphylococcus aureus (MR) 918019 | 4 | 8/152 | 1/2/38 | 0.5 | S |
| Staphylococcus aureus (MR) 903887 | 8 | 4/76 | 1/1/19 | 0.38 | S |
| Staphylococcus aureus (MR) 974980 | 8 | 4/76 | 1/1/19 | 0.38 | S |
| Staphylococcus aureus (MR) 1045190 | 4 | 4/76 | 1/1/19 | 0.5 | S |
| Staphylococcus aureus (MR) 927596 | 8 | >8/>152 | 1/4/76 | ≤0.38 | S |
| Staphylococcus aureus (MR) 981550 | 4 | 8/152 | 1/2/38 | 0.5 | S |
| Gram [symbol] negative | | | | | |
| Escherichia coli ATCC 25922 | 2 | >8/>152 | 0.5/2/38 | ≤0.38 | S |
| Escherichia coli 863696 | 8 | >8/>152 | 2/4/76 | ≤0.5 | S |
| Escherichia coli 977941 | 4 | 8/152 | 1/2/38 | 0.5 | S |
| Escherichia coli 846450 | 4 | 4/76 | 1/1/19 | 0.5 | S |
| Escherichia coli 854566 | 16 | >8/>152 | 4/4/76 | ≤0.5 | S |
| Escherichia coli 996332 | 4 | >8/>152 | 1/2/38 | ≤0.38 | S |
| Escherichia coli 961128 | 2 | >8/>152 | 0.5/2/38 | ≤0.38 | S |
| Escherichia coli 857021 | 2 | >8/>152 | 32/4/76 | ≤0.31 | S |
| Escherichia coli 975811 | 4 | >8/>152 | 1/4/76 | ≤0.5 | S |
| Escherichia coli 959117 | 2 | >8/>152 | 0.5/4/76 | ≤0.5 | S |
| Escherichia coli 976657 | 4 | 8/152 | 1/2/38 | 0.5 | S |
| Escherichia coli 863678 | 2 | >8/>152 | 0.5/4/76 | 0.5 | S |
| Escherichia coli 846446 | 2 | 4/76 | 0.5/1/19 | 0.5 | S |
| Escherichia coli 928017 | 2 | >8/>152 | 0.5/2/38 | ≤0.38 | S |
| Klebsiella pneumoniae 857147 | 32 | >8/>152 | 8/4/76 | ≤0.5 | S |
| Klebsiella pneumoniae 957467 | 16 | >8/>152 | 4/4/76 | ≤0.5 | S |
| Klebsiella pneumoniae 993434 | 16 | >8/>152 | 4/4/76 | ≤0.5 | S |
| Klebsiella pneumoniae 869028 | 8 | >8/>152 | 2/4/76 | ≤0.5 | S |
| Klebsiella pneumoniae 848788 | 32 | >8/>152 | 4/4/76 | ≤0.38 | S |
| Klebsiella pneumoniae 870964 | 8 | >8/>152 | 2/4/76 | ≤0.5 | S |
| Klebsiella pneumoniae 926439 | 4 | >8/>152 | 1/4/76 | ≤0.5 | S |
| Klebsiella pneumoniae 875638 | 16 | >8/>152 | 4/2/38 | ≤0.5 | S |
| Klebsiella pneumoniae 957464 | 16 | >8/>152 | 4/4/76 | ≤0.5 | S |
| Klebsiella pneumoniae 926456 | 64 | >8/>152 | 1/2/38 | ≤0.14 | S |
| Klebsiella pneumoniae 984643 | 16 | >8/>152 | 1/4/76 | ≤0.31 | S |
| Klebsiella pneumoniae 939665 | 64 | >8/>152 | 1/2/38 | ≤0.14 | S |
| Klebsiella pneumoniae 868968 | 8 | >8/>152 | 2/2/38 | ≤0.38 | S |
| Klebsiella pneumoniae 995460 | 16 | >8/>152 | 4/4/76 | ≤0.5 | S |
| Klebsiella pneumoniae 1040205 | 16 | >8/>152 | 4/4/76 | ≤0.5 | S |
| Klebsiella pneumoniae 892483 | 4 | >8/>152 | 1/2/38 | ≤0.38 | S |
| Pseudomonas aeruginosa 880238 | 8 | >8/>152 | 2/4/76 | ≤0.5 | S |
| Pseudomonas aeruginosa 889058 | 64 | >8/>152 | 16/4/76 | ≤0.5 | S |
| Pseudomonas aeruginosa 906939 | 64 | >8/>152 | 16/2/38 | ≤0.38 | S |
| Pseudomonas aeruginosa 914881 | 128 | >8/>152 | 16/4/76 | ≤0.38 | S |
| Acinetobacter baumannii 843978 | 8 | 2/38 | 1/0.5/9.5 | 0.38 | S |
| Acinetobacter baumannii 918857 | 64 | >8/>152 | 16/2/38 | ≤0.38 | S |
| Acinetobacter baumannii 919656 | 8 | 2/38 | 1/0.5/9.5 | 0.38 | S |
| Acinetobacter baumannii 919899 | 8 | 2/38 | 1/0.5/9.5 | 0.38 | S |
| Acinetobacter baumannii 938781 | 16 | 4/76 | 1/1/19 | 0.31 | S |
| Acinetobacter baumannii 1049160 | 8 | 2/38 | 1/0.5/9.5 | 0.38 | S |

Q = alternate bacterial DHFR inhibitor, SCH79797; S = sulfamethoxazole (a sulfonamide); MR = methicillin resistant. [symbol]
indicates data missing or illegible when filed

TABLE 10C
Synergy of antimicrobial duo (Q, S) against
facultative anaerobic pathogens.

| | Q | S | Q/S | FIC | Interpretation |
|---|---|---|---|---|---|
| Staphylococcus aureus | 32 | >608 | 8/152 | ≤0.5 | S |
| (MR) 920516 | | | | | |
| Escherichia coli 846450 | >32 | >608 | 4/76 | ≤0.13 | S |
| Escherichia coli 976657 | >32 | 608 | 8/152 | ≤0.38 | S |
| Escherichia coli 846446 | >32 | >608 | 4/76 | ≤0.13 | S |

Q = bacterial DHFR inhibitor, SCH79797; S = sulfamethoxazole (a sulfonamide).

TABLE 11
Further (better) synergy of antimicrobial combination (F, D, S)
with a further co-agent (PGSI + BLI) against obligate anaerobic pathogens.

| | MIC (μg/ml) | | | | | |
|---|---|---|---|---|---|---|
| Gram-positive | F | D/S | A-C | F/D/S/A-C | FIC | Interpretation |
| Clostridium perfringens 12-117 | 32 | >32/>608 | 1 | 4/2/38/0.25 | ≤0.41 | S |
| Clostridium perfringens 11-46 | 32 | >32/>608 | 8 | 8/2/38/1 | ≤0.41 | S |
| Clostridium perfringens 11-50 | 8 | 16/304 | 0.125 | 0.5/0.06/1.19/0.03 | 0.32 | S |
| Clostridium perfringens 11-61 | 16 | 32/608 | 3 | 0.5/2/38/0.25 | 0.34 | S |
| Clostridium perfringens 2-0246-4 | 16 | >32/>608 | 0.5 | 0.5/0.06/1.19/0.125 | 0.28 | S |
| Clostridioides difficile 3/8 | 32 | 32/608 | 0.5 | 0.5/0.06/1.19/0.125 | 0.27 | S |
| Clostridioides difficile 2-0247-4 | 32 | >32/>608 | 1 | 0.5/1/19/0.25 | 0.28 | S |
| Clostridioides difficile 10-3620-5 | 16 | >82/>608 | 0.25 | 0.5/0.06/1.19/0.06 | 0.28 | S |
| Bifidobacterium animalis TBC0002 | 64 | >32/>608 | 8 | 0.5/2/38/0.25 | ≤0.07 | S |
| Peptostreptococcus anaerobious 807256 | 16 | >32/>608 | 4 | 1/0.25/4.75/0.03 | 0.1 | S |
| Cutibacterium acnes 6609 | 64 | 8/152 | 8 | 0.5/0.06/1.19/0.125 | 0.03 | S |
| Gram-negative | | | | | | |
| Bacteroides fragilis ATCC 23745 | >128 | 1/19 | 1 | 0.5/0.06/1.19/0.25 | 0.26 | S |
| Bacteroides fragilis ATCC 25285 | >128 | 2/38 | 1 | 1/0.5/9.5/0.03 | 0.31 | S |
| Bacteroides fragilis 016M | >128 | 4/76 | 2 | 1/1/19/0.25 | ≤038 | S |
| Bacteroides fragilis 638R | >128 | 2/38 | 1 | 0.5/0.06/1.19/0.25 | 0.28 | S |
| Bacteroides thetaiotaomicron VPI 5842 | 128 | 8/152 | 1 | 0.5/0.06/1.19/0.25 | 0.26 | S |
| Bacteroides ovatus ATCC 8483 | >128 | 4/76 | 2 | 0.5/0.06/1.19/0.25 | ≤0.14 | S |
| Phacoeicola vulgatus TCC 8482 | >128 | 0.5/9.5 | 1 | 0.5/0.06/1.19/0.25 | ≤0.37 | S |
| Prevotella intermedia 8071621 | >128 | 0.25/4.75 | 0.5 | 0.8/0.06/1.19/0.06 | ≤0.5 | S |
| Bacteroides fragilis CLA 267 | >128 | 0.25/4.75 | 2 | 0.5/0.06/1.19/0.25 | ≤0.37 | S |
| Phacoeicola vulgatus ATCC 29327 | 128 | 0.25/2.38 | 0.5 | 0.5/0.06/1.19/0.03 | 0.31 | S |
| Parabacteroides distasonis CLA 348 | >128 | 0.25/4.75 | 0.25 | 0.5/0.06/1.19/0.03 | ≤0.38 | S |

D = trimethoprim (a diaminopyridine); S = sulfamethoxazole (a sulfonamide).
PGSI + BLI = amoxicillin-clavulanate (A-C)

TABLE 12
Further (better) synergy of antimicrobial combination (F, D, S)
with a further co-agent (PGSI) against obligate anaerobic pathogens.

| | MIC (μg/ml) | | | | | |
|---|---|---|---|---|---|---|
| Gram-positive | F | D/S | V | F/D/S/V | FIC | Interpretation |
| Clostridium perfringens 12-117 | 16 | >32/>608 | 2 | 2/0.03/0.25 | ≤0.25 | S |
| Clostridium perfringens 11-61 | 16 | >32/>608 | 0.5 | 2/0.03/0.125 | ≤0.38 | S |
| Clostridium perfringens 2-0246-4 | 4 | >32/>608 | 2 | 2/0.03/0.5 | ≤0.38 | S |
| Clostridioides difficile 2-0247-4 | 8 | >32/>608 | 0.5 | 2/0.03/0.06 | ≤0.37 | S |
| Bifidobacterium animalis TBC0002 | 32 | 16/304 | 2 | 2/0.03/0.5 | 0.31 | S |
| Peptostreptococcus anaerobious 807256 | 16 | >32/>608 | 2 | 2/0.03/0.5 | ≤0.38 | S |
| Cutibacterium acnes 6609 | 64 | 32/608 | 2 | 2/0.03/0.5 | 0.31 | S |
| Gram-negative | | | | | | |
| Bacteroides fragilis ATCC 25285 | 64 | 2/38 | 0.5 | 2/0.03/0.125 | 0.3 | S |
| Bacteroides fragilis 016M | >128 | 1/19 | 0.25 | 2/0.03/0.06 | ≤0.29 | S |
| Bacteroides thetaiotaomicron VPI 5842 | >128 | 4/76 | 2 | 2/0.03/0.5 | ≤0.27 | S |

D = trimethoprim (a diaminopyridine); S = sulfamethoxazole (a sulfonamide).
PGSI = vancomycin (V).

TABLE 13
Lack of synergy of antimicrobial combination (F, D, S) against
beneficial or mutualistic facultative anaerobic bacteria.

| | MIC (μg/ml) | | |
|---|---|---|---|
| Gram-positive | F | D/S | F/D/S |
| Lactobacillus acidophilus JP0002 | >128 | >32/>608 | >64/>32/>608 |
| (O2 tolerant) | | | |
| Lactobacillus delbrueckii JP0006 | >128 | >32/>608 | >64/>32/>608 |
| Lactobacillus gasseri JP0032 | >128 | >32/>608 | >64/>32/>608 |
| Lactobacillus paracasei JP0032 | >128 | >32/>608 | >64/>32/>608 |
| Lactobacillus plantarum JP0036 | >128 | >32/>608 | >64/>32/>608 |
| Leuconostoc mesenteroides JP0037 | >128 | >32/>808 | >64/>32/>608 |
| Pediococcus acidilactici JP0068 | >128 | >32/>608 | >64/>32/>608 |
| Pediococcus pentosaceus JP0068 | >128 | >32/>608 | >64/>32/>608 |

D = trimethoprim (a diaminopyridine); S = sulfamethoxazole (a sulfonamide).

TABLE 14A
Further (better) synergy of antimicrobial combination (F, D, S) with a further co-agent
(FMEI) against facultative anaerobic pathogens (continued In Table 14B)

| | MIC (μg/ml) | | | | | | |
|---|---|---|---|---|---|---|---|
| Gram-positive | F | F + P | D/S | D + S/P | F/D/S | FIC^{a} | Interpretation |
| Staphylococcus aureus 927596 | 8 | 1 | 0.25/4.75 | 0.25/4.75 | 1/0.06/1.19 | 0.38 | S |
| Staphylococcus aureus 829025 | 8 | 1 | 0.25/4.75 | 0.25/4.75 | 1/0.06/1.19 | 0.38 | S |
| Staphylococcus aureus 956270 | 8 | 2 | 0.25/4.75 | 0.25/4.75 | 1/0.06/1.19 | 0.38 | S |
| Staphylococcus aureus 1045190 | 8 | 2 | 0.25/4.75 | 0.25/4.75 | 1/0.03/0.6 | 0.25 | S |
| Staphylococcus aureus 981550 | 8 | 1 | 0.25/4.75 | 0.25/4.75 | 1/0.03/0.6 | 0.25 | S |
| Staphylococcus aureus 920576 | 8 | 1 | 0.25/4.75 | 0.25/4.75 | 1/0.06/1.19 | 0.38 | S |
| Staphylococcus aureus 920962 | 8 | 1 | 0.25/4.75 | 0.25/4.75 | 1/0.03/0.6 | 0.25 | S |
| Staphylococcus aureus 952566 | 8 | 2 | 0.25/4.75 | 0.25/4.75 | 1/0.03/0.6 | 0.25 | S |
| Staphylococcus aureus 956362 | 8 | 2 | 0.25/4.75 | 0.25/4.75 | 1/0.03/0.6 | 0.25 | S |
| Staphylococcus aureus 894946 | 8 | 2 | 0.25/4.75 | 0.25/4.75 | 1/0.03/0.6 | 0.25 | S |
| Gram-negative | | | | | | | |
| Escherichia coli 854535 | 4 | 2 | 1/19 | 1/19 | 1/0.25/4.75 | 0.5 | S |
| Escherichia coli 976657 | 16 | 4 | 1/19 | 1/19 | 1/0.25/4.75 | 0.31 | S |

TABLE 14B
Continuation of Table 14A.

| | MIC (μg/ml) | | |
|---|---|---|---|
| | P + F/D/S | FIC^{b} | Interpretation |
| Gram-positive | | | |
| Staphylococcus aureus 927596 | 1/0.015/.3 | 0.19 | S |
| Staphylococcus aureus 829025 | 1/0.015/.3 | 0.19 | S |
| Staphylococcus aureus 956270 | 1/0.015/.3 | 0.19 | S |
| Staphylococcus aureus 1045190 | 1/0.015/0.3 | 0.19 | S |
| Staphylococcus aureus 981550 | 1/0.015/0.3 | 0.19 | S |
| Staphylococcus aureus 920576 | 1/0.015/0.3 | 0.19 | S |
| Staphylococcus aureus 920962 | 1/0.015/0.3 | 0.19 | S |
| Staphylococcus aureus 952566 | 1/0.015/0.3 | 0.19 | S |
| Staphylococcus aureus 956362 | 1/0.015/0.3 | 0.19 | S |
| Staphylococcus aureus 894946 | 1/0.015/0.3 | 0.19 | S |
| Gram-negative | | | |
| Escherichia coli 854535 | 1/0.06/1.19 | 0.31 | S |
| Escherichia coli 976657 | 1/0.06/1.19 | 0.12 | S |

### FOR Tables 14A and 14B:

D=trimethoprim (a diaminopyridine); S=sulfamethoxazole (a sulfonamide).
FMEI=phosphonoformate (P)
^{a}FIC=fractional inhibitory concentration for F/D/S
^{a}FIC=fractional inhibitory concentration for P+F/D/S.

TABLE 15
Further (better) synergy of antimicrobial combination (F, D, S) with a
further co-agent (PGSI or PGSI + BLI) against facultative anaerobic pathogens.

| | MIC (μg/ml) | | | | | | |
|---|---|---|---|---|---|---|---|
| Gram-positive | F | D/S | F/D/S | PGSI | F/D/S/PGSI | FIC^{a}//FIC^{b} | Interpr. |
| Staphylococcus aureus ATCC 33591^{c} | 4 | 1/19 | 1/0.25/4,75 | 2 | 1/0.0039/0.075/0.125 | 0.5//0.39 | S/S |
| Staphylococcus aureus ATCC 33591^{d} | 4 | 1/19 | 1/0.25/4.75 | 0.125 | 1/0.125/2.38/0.03 | 0.5//0.5 | S/S |
| Staphylococcus aureus 953041^{c} | 4 | 0.06/1.19 | 1/0.015/0.3 | 1 | 0.5/0.015/0.3/0.125 | 0.5//0.5 | S/S |
| Staphylococcus aureus 953041^{d} | 4 | 0.06/1.19 | 1/0.015/0.3 | 0.25 | 0.25/0.015/0.3/0.03 | 0.5//0.44 | S/S |
| Staphylococcus aureus 952566^{d} | >16 | 0.06/1.19 | 4/0.015/0.3 | 0.5 | 2/≤0.00048/≤0.095/0.13 | 0.38//<0.32 | S/S |
| Staphylococcus aureus 981550^{d} | 16 | 0.06/1.19 | 2/0.015/0.3 | 0.5 | 2/0.0039/0.075/0.03 | 0.38//0.25 | S/S |
| Staphylococcus aureus 994946^{d} | 2 | 0.06/1.19 | 0.5/0.015/0.3 | 0.25 | 0.125/0.015/0.3/0.03 | 0.5//0.43 | S/S |
| Staphylococcus aureus 1022425^{d} | 16 | 0.125/2.38 | ≤0.25/0.03/0.6 | 0.25 | ≤0.25/≤0.0019/≤0.038/0.03 | ≤0.27//≤0.16 | S/S |
| Enterococcus faecalis 1086932^{e} | >128 | 1/19 | 16/0.25/4.75 | 1 | ≤2/0.125/2.38/0.125 | ≤0.31//≤0.26 | S/S |
| Enterococcus faecalis 1086932^{c} | >128 | 1/19 | 16/0.25/4.75 | 1 | ≤2/0.125/2.38/0.25 | ≤0.31//≤0.38 | S/S |
| Enterococcus faecalis ATCC 51559^{e} | >128 | 0.5/9.5 | 4/0.125/2.38 | >16 | 4/0.06/1.19/4 | ≤0.27//≤0.26 | S/S |
| Enterococcus faecalis ATCC 51559^{c} | >128 | 0.5/9.5 | 4/0.125/2.38 | >32 | <2/0.06/1.19/8 | ≤0.27//≤0.25 | S/S |
| Gram-negative | | | | | | | |
| Escherichia coli ATCC 25922^{f} | 1 | 0.25/4.75 | 0.06/0.06/1.19 | 4 | 0.03/0.015/0.3/0.125 | 0.31//0.12 | S/S |
| Escherichia coli 846446^{f} | 1 | 0.5/9.5 | 0.06/0.03/0.6 | >4 | ≤0.015/0.015/0.3/1 | 0.12//≤0.17 | S/S |
| Klebsiella pneumoniae 870964^{f} | 64 | 8/152 | 16/1/19 | 4 | 4/0.06/1.19/0.5 | 0.38//0.2 | S/S |
| Klebsiella pneumoniae 875638^{f} | 64 | >32/608 | 16/0.5/9.5 | >4 | 4/0.25/4.75/1 | ≤0.26//≤0.19 | S/S |
| Pseudomonas aeruginosa 945748^{f} | 128 | 32/608 | 32/1/19 | 2 | 8/0.25/4.75/0.125 | 0.28//0.13 | S/S |
| Pseudomonas aeruginosa 945748^{g} | 128 | 32/608 | 32/1/19 | 8 | 8/0.25/4.75/0.125 | 0.28//0.13 | S/S |
| Acinetobacter baumannii 919899^{f} | >128 | >32/>608 | 8/1/19 | >8 | 8/0.25/4.75/1 | 0.05//0.1 | S/S |
| Acinetobacter baumannii 919899^{g} | >128 | >32/>608 | 8/1/19 | >64 | 8/0.25/4.75/0.25 | 0.05//0.04 | S/S |

D = trimethoprim (a diaminopyridine); S = sulfamethoxazole (a sulfonamide).
^{a}FIC = fractional inhibitory concentration for F/D/S
^{b}FIC = fractional inhibitory concentration for F/D/S/PGI
^{c}PGSI tested = vancomycin.
^{d}PGSI tested = telavancin.
^{e}PGSI and BLI tested = amoxicillin-clavulanate.
^{f}PGSI and BLI tested = ceftazidime-avibactam.
^{g}PGSI = meropenem

TABLE 16
Further (better) synergy of antibacterial combination (F, D, S) with a further co-agent (PGSI or PGSI + BLI) against
facultative anaerobic pathogens also unexpectedly enhances post-antibiotic effect (PAE) of antibacterial combination.

| | F | PAE | D/S | PAE | V | PAE | F/D/S | PAE | F/D/S/V | PAE |
|---|---|---|---|---|---|---|---|---|---|---|
| Gram-positive | (μg/ml) | (hrs) | (μg/ml) | (hrs) | (μg/ml) | (hrs) | (μg/ml) | (hrs) | (μg/ml) | (hrs) |
| Staphylococcus aureus ATCC 33591^{c} | 16 | 0.5 | 1/19 | 0.25 | 4 | 1 | 4/0.25/4.75 | 0.5 | 2/0.125/2.38/0.5 | 0.5 |
| Staphylococcus aureus ATCC 33591^{c} | 32 | 0.5 | 2/38 | 0.25 | 8 | 1 | 8/0.5/9,5 | 0.5 | 4/0.25/4.75/1 | 1 |
| Staphylococcus aureus ATCC 33591^{c} | 64 | 0.5 | 4/64 | 0.25 | 16 | 2 | 16/1/19 | 1 | 8/0.5/9.5/2 | 1.5 |
| | F | PAE | D/S | PAE | CAv | PAE | F/D/S | PAE | F/D/S/CAv | PAE |
| Gram-negative | (μg/ml) | (hrs) | (μg/ml) | (hrs) | (μg/ml) | (hrs) | (μg/ml) | (hrs) | (μg/ml) | (hrs) |
| Escherichia coli ATCC 25922 | 1 | 0.5 | 0.5/9.5 | 0.5 | 4/8 | 0 | 0.125/0.125/ | 0.5 | 0.06/0.03/0.6/ | 0.5 |
| | | | | | | | 2.38 | | 4/0.25 | |
| Escherichia coli ATCC 25922 | 2 | 0.5 | 1/19 | 0.5 | 4/16 | 0 | 0.25/0.25/ | 1 | 0.125/0.06/1.19/ | 1 |
| | | | | | | | 4.75 | | 4/0.5 | |
| Escherichia coli ATCC 25922 | 4 | 1 | 2/38 | 0.5 | 4/32 | 0 | 0.5/0.5/9.5 | 1 | 0.25/0.125/2.38/ | 1.5 |
| | | | | | | | | | 4/1 | |
| Escherichia coli ATCC 25922 | 8 | 1.5 | | | | | | | | |

^{c}Beta-lactam resistant strains.
D = trimethoprim (a diaminopyridine); S = sulfamethoxazole (a sulfonamide).
PGSI = vancomycin (V).
PGSI + BLI = ceftazidime-avibactam (CAv)

TABLE 17
Evidence of unpredictability as shown with a representative
antimicrobial combination (F, D, S) against representative
pathogenic obligate anaerobes.

| | MIC (μg/ml) | | F/D/S | |
|---|---|---|---|---|
| | F | D/S | Interpretation | FIC |
| Gram-positive | | | | |
| Clostridium perfringens 12-117 | + | −/− | S | 0.27 |
| Clostridium perfringens 11-46 | + | −/− | S | 0.12 |
| Clostridium perfringens 11-50 | + | −/− | S | 0.19 |
| Clostridium perfringens 11-61 | + | −/− | S | 0.25 |
| Clostridium perfringens 2-0246-4 | + | −/− | S | 0.06 |
| Clostridioides difficile 3/8 | + | −/− | S | 0.28 |
| Clostridioides difficile 2-0247-4 | + | −/− | S | 0.27 |
| Clostridioides difficile 10-3620-5 | + | −/− | S | 0.28 |
| Clostridium septicum 11-3571 | + | −/− | S | <0.13 |
| Clostridium septicum 15-847 | + | −/− | S | 0.31 |
| Bifidobacterium animalis JLP-002 | − | +/+ | S | <0.07 |
| Peptostreptococcus anaerobious | + | −/− | S | <0.5 |
| 807256 | | | | |
| Cutibacterium acnes 6609 | + | −/− | S | 0.04 |
| Gram-negative | | | | |
| Bacteroides fragilis ATCC 23745 | − | −/− | S | 0.25 |
| Bacteroides fragilis ATCC 25285 | − | −/− | S | 0.25 |
| Bacteroides fragilis CLA267 | − | −/− | S | 0.25 |
| Bacteroides fragilis O16M | − | −/− | S | 0.25 |
| Bacteroides fragilis 638R | − | −/− | S | 0.25 |
| Bacteroides ovatus ATCC 8483 | − | −/− | S | 0.25 |
| Bacteroides thetaiotaomicron | − | −/− | S | 0.25 |
| VPI5482 | | | | |
| Phocaeicola vulgatus ATCC 8482 | − | −/− | S | 0.25 |
| Phocaeicola vulgatus ATCC 29327 | − | −/− | S | 0.25 |

D = trimethoprim (w/trimethoprim as a representative D, for extrapolated MIC breakpoint, as there is no breakpoint against anaerobes).
S = sulfamethoxazole (w/sulfamethoxazole as representative S, for extrapolated MIC breakpoint, as there is no breakpoint against anaerobes).
+ = when MIC is at or below a clinical breakpoint (anaerobe is susceptible to tested antibiotic(s): F, or D and S; where D and S are co-tested).
− = when MIC is above a clinical breakpoint (anaerobe is not susceptible to tested antibiotic(s): F, or D and S; where D and S are co-tested).

TABLE 18
Representative susceptibility data for a representative antimicrobial combination
(F, D, S) against representative pathogenic bacteria at acidic pH (pH 5).

| | MIC (mg/ml) | | | | |
|---|---|---|---|---|---|
| | F | D/S | F/D/S | FIC | Interpretation |
| Gram-positive | | | | | |
| Staphylococcus aureus 33591 | 32 | 2/38 | 0.06/0.5/9.5 | 0.25 | S |
| Staphylococcus aureus 102485 | 32 | 0.5/9.5 | 0.06/0.125/2.38 | 0.25 | S |
| Staphylococcus aureus 953041 | 32 | 0.5/9.5 | 0.06/0.06/1.19 | 0.12 | S |
| Enterococcus faecalis 108 | 8 | 2/38 | 0.03/0.5/9.5 | 0.26 | S |
| Enterococcus faecium 51559 | 16 | >32/>608 | 0.5/0.0078/0.15 | 0.03 | S |
| Bacillus cereus ATCC | 4 | 0.5/9.5 | 0.06/0.03/0.6 | 0.08 | S |
| Bacillus subtilis 6633 | 64 | 0.25/4.75 | 0.06/0.03/0.6 | 0.13 | S |
| Gram-negative | | | | | |
| Escherichia coli 25922 | 2 | 1/19 | 0.5/0.03/0.6 | 0.28 | S |
| Escherichia coli 857021 | 2 | >32/>608 | 0.5/0.0078/0.15 | 0.26 | S |
| Escherichia coli 847021 | 32 | >32/>608 | 8/4/76 | 0.31 | S |
| Escherichia coli CDC-0089 | 8 | 8/152 | 2/0.06/1.19 | 0.26 | S |
| Pseudomonas aeruginosa 27853 | 32 | 2/38 | 0.06/0.25/4.75 | 0.13 | S |
| Pseudomonas aeruginosa 949708 | 128 | 32/608 | 32/4/76 | 0.38 | S |
| Pseudomonas aeruginosa 975744 | 32 | 16/304 | 4/1/19 | 0.19 | S |
| Klebsiella pneumoniae 848788 | 64 | >32/>608 | 16/8/152 | 0.38 | S |
| Klebsiella pneumoniae 939665 | >128 (256) | 2/38 | 0.06/0.5/9.5 | 0.26 | S |
| Acinetobacter baumannii 919656 | 32 | 16/304 | 0.06/1/19 | 0.06 | S |
| Acinetobacter baumannii 918857 | 16 | 0.5/9.5 | 4/0.06/1.19 | 0.37 | S |
| Burkholderia thailandensis ATCC | >128 (256) | 2/38 | 0.06/0.06/1.19 | 0.03 | S |
| Yersinia pseudotuberculosis ATCC | 2 | 0.5/9.5 | 0.03/0.125/2.38 | 0.27 | S |

F = fosfomycin sodium; D = trimethoprim (a diaminopyridine); S = sulfamethoxazole (a sulfonamide).

TABLE 19
Representative susceptibility data for a representative antimicrobial combination
(F, D, S) against representative pathogenic bacteria at neutral pH (pH 7).

| | MIC (mg/ml) | | | | |
|---|---|---|---|---|---|
| | F | D/S | F/D/S | FIC | Interpretation |
| Gram-positive | | | | | |
| Staphylococcus aureus 33591 | 4 | 4/76 | 0.06/0.5/9.5 | 0.14 | S |
| Staphylococcus aureus 102485 | 4 | 4/76 | 0.06/0.06/1.19 | 0.03 | S |
| Staphylococcus aureus 953041 | 4 | 0.5/9.5 | 0.06/0.06/1.19 | 0.14 | S |
| Enterococcus faecalis 1086432 | 64 | 0.25/4.75 | 0.06/0.015/0.3 | 0.06 | S |
| Enterococcus faecium 51559 | 128 | 32/608 | 32/2/38 | 0.21 | S |
| Bacillus cereus ATCC 14579 | 64 | 0.5/9.5 | 0.06/0.015/0.3 | 0.03 | S |
| Bacillus subtilis ATCC 6633 | >128 | 0.125/1.19 | 4/0.03/0.6 | 0.27 | S |
| Gram-negative | | | | | |
| Escherichia coli 25922 | 8 | 2/38 | 0.06/0.5/9.5 | 0.26 | S |
| Escherichia coli 857021 | 32 | >32/>608 | 0.06/0.5/9.5 | 0.01 | S |
| Escherichia coli 847021 | 64 | >32/608 | 16/0.03/0.6 | 0.26 | S |
| Escherichia coli CDC-0089 | 2 | 8/152 | 0.06/2/38 | 0.28 | S |
| Pseudomonas aeruginosa 27853 | 128 | 32/608 | 8/0.25/4.75 | 0.07 | S |
| Pseudomonas aeruginosa 949708 | 128 | >32/>608 | 16/0.25/4.75 | 0.13 | S |
| Pseudomonas aeruginosa 975744 | 128 | >32/>608 | 32/0.25/4.75 | 0.26 | S |
| Klebsiella pneumoniae 848788 | 128 | >32/>608 | 32/1/19 | 0.27 | S |
| Klebsiella pneumoniae 939665 | 128 | 1/19 | 8/0.25/4.75 | 0.31 | S |
| Acinetobacter baumannii 919656 | >128 | 8/152 | 32/2/38 | 0.38 | S |
| Acinetobacter baumannii 918857 | 64 | 2/38 | 16/0.5/9.5 | 0.5 | S |
| Burkholderia thailandensis ATCC 700388 | 64 | 0.5/9.5 | 8/0.125/2.38 | 0.38 | S |
| Yersinia pseudotuberculosis ATCC 6905 | 64 | 0.25/4.75 | 4/0.0078/0.15 | 0.09 | S |

F = fosfomycin sodium; D = trimethoprim (a diaminopyridine); S = sulfamethoxazole (a sulfonamide).

TABLE 20
Representative susceptibility data for a representative antimicrobial combination
(F, D, S) against representative pathogenic bacteria at basic pH (pH 8).

| | MIC (mg/ml) | | | | |
|---|---|---|---|---|---|
| | F | D/S | F/D/S | FIC | Interpretation |
| Gram-positive | | | | | |
| Staphylococcus aureus 33591 | 8 | 4/76 | 0.5/0.5/9.5 | 0.19 | S |
| Staphylococcus aureus 102485 | 8 | 2/38 | 0.5/0.125/2.38 | 0.13 | S |
| Staphylococcus aureus 953041 | 4 | 2/38 | 0.5/0.5/9.5 | 0.38 | S |
| Enterococcus faecalis 1086432 | 128 | 0.25/4.75 | 0.06/0.03/0.6 | 0.12 | S |
| Enterococcus faecium 51559 | 64 | 32/608 | 0.5/2/38 | 0.06 | S |
| Bacillus cereus ATCC 14579 | 32 | 4/76 | 0.5/1/19 | 0.27 | S |
| Bacillus subtilis ATCC 6633 | >256 | 0.125/2.38 | ≤0.03/0.03/0.6 | 0.26 | S |
| Gram-negative | | | | | |
| Escherichia coli 25922 | 8 | 8/152 | 0.5/0.5/9.5 | 0.13 | S |
| Escherichia coli 857021 | 4 | >32/>608 | 0.25/0.125/2.38 | 0.13 | S |
| Escherichia coli 847021 | 128 | >32/>608 | 0.03/16/304 | 0.27 | S |
| Escherichia coli CDC-0089 | 64 | 4/76 | 16/1/19 | 0.5 | S |
| Pseudomonas aeruginosa 27853 | 64 | 32/608 | 16/8/152 | 0.5 | S |
| Pseudomonas aeruginosa 949708 | 128 | >32/>608 | 8/16/304 | 0.31 | S |
| Pseudomonas aeruginosa 975744 | >256 | >32/>608 | 0.03/16/304 | 0.26 | S |
| Klebsiella pneumoniae 848788 | >256 | >32/>608 | 0.5/0.5/9.5 | 0.01 | S |
| Klebsiella pneumoniae 939665 | >256 | 0.5/9.5 | ≤0.03/0.125/2.38 | 0.26 | S |
| Acinetobacter baumannii 919656 | 256 | 16/304 | 0.5/0.5/9.5 | 0.03 | S |
| Acinetobacter baumannii 918857 | NG | NG | NG | ND | ND |
| Burkholderia thailandensis ATCC 700388 | NG | NG | NG | ND | ND |
| Yersinia pseudotuberculosis ATCC 6905 | >256 | 0.25/4.75 | 0.5/0.06/1.19 | 0.26 | S |

F = fosfomycin sodium; D = trimethoprim (a diaminopyridine); S = sulfamethoxazole (a sulfonamide).
NG = no growth; ND = not determined.

TABLE 21
Summary of susceptibility (and [symbol] ynergy) for a repres [symbol] ntative antimicrobia [symbol] combination
(F, D, S) [symbol] gainst representative pathogenic bacteria at acidic pH, neutral pH, and basi [symbol] pH.

| | F/D/S (μg/ml) | | |
|---|---|---|---|
| | pH 5 | pH 7 | pH 8 |
| Gram-positive | | | |
| Staphylococcus aureus 33591 | 0.06/0.5/9.5 | 0.06/0.5/9.5 | 0.5/0.5/9.5 |
| Staphylococcus aureus 102485 | 0.06/0.135/2.38 | 0.06/0.06/1.19 | 0.5/0.125/2.38 |
| Staphylococcus aureus 953041 | 0.06/0.06/1.19 | 0.06/0.06/1.19 | 0.5/0.5/9.5 |
| Enterococcus faecalis 1086432 | 0.03/0.5/9.5 | 0.06/0.015/0.3 | 0.06/0.03/0. [symbol] |
| Enterococcus faecium 51559 | 0.5/0.00 [symbol] 8/0.15 | 32/2/38 | 0.5/2/38 |
| Bacillus cereus ATCC 14579 | 0.06/0.03/0.6 | 0.06/0.015/0.3 | 0.5/1/19 |
| Bacillus subtilis ATCC 6633 | 0.06/0.03/0.6 | 4/0.03/0.6 | ≤0.03/0.03/0.6 |
| Gram-negative | | | |
| Escherichia coli 25922 | 0.5/0.03/0.6 | 0.06/0.5/9.5 | 0.5/0.5/9.5 |
| Escherichia coli 857021 | 0.5/0.00 [symbol] 8/0.15 | 0.06/0.5/9.5 | 0.25/0.125/2.38 |
| Escherichia coli 847021 | 8/4/76 | 16/0.03/0.6 | 0.03/16/304 |
| Escherichia coli CDC-0089 | 2/0.06/1.19 | 0.06/2/38 | 16/2/38 |
| Pseudomonas aeruginosa 27853 | 0.06/0.25/4.75 | 8/0.25/4.75 | 16/8/152 |
| Pseudomonas aeruginosa 949708 | 32/4/76 | 16/0.25/4.75 | 8/32/608 |
| Pseudomonas aeruginosa 975744 | 4/1/19 | 32/0.25/4.75 | 0.03/16/304 |
| Klebsiella pneumoniae 848788 | 16/8/152 | 32/1/19 | 0.5/0.5/9.5 |
| Klebsiella pneumoniae 939665 | 0.06/0.5/9.5 | 8/0.25/4.75 | <0.03/0.25/4.75 |
| Acinetobacter baumannii 919656 | 0.06/ [symbol] /19 | 32/2/38 | 0.5/0.5/9.5 |
| Acinetobacter baumannii 918857 | 4/0.06/1.19 | 16/0.5/9.5 | NG |
| Burkholderia thailandensis ATCC 700388 | 0.06/0.06/1.19 | 8/0.125/2.38 | NG |
| Yersinia pseudotuberculosis ATCC 6905 | 0.03/0.1 [symbol] 5/2.38 | 4/0.0078/0.15 | 0.5/0.25/4.75 |

F = fosfomycin sodium; D = trimethoprim (a diaminopyridine); S = sulfamethoxazole (a sulfonamide).
NG = no growth. [symbol]
indicates data missing or illegible when filed

What is claimed includes:

1. A plurality of co-agents for utilization as an antimicrobial medicament against one or more pathogenic anaerobes in an anaerobic environment, the plurality of co-agents comprising:
a fosfomycin in a pharmaceutically acceptable form selected from one or more of a salt, phosphate, acid, amine, and ester, in which the fosfomycin alone is active against an aerobic bacteria in an aerobic environment, and may be substituted for an inhibitor of bacterial UDP-GlcNAc enolpyruvyl transferase that has some activity against an aerobic bacteria in an aerobic environment;
a diaminopyridine in a pharmaceutically acceptable form selected from one or more of a salt, phosphate, acid, and ester, in which the diaminopyridine is active against an aerobic bacteria in an aerobic environment, and may be substituted for an inhibitor of bacterial dihydrofolate reductase that has some activity against an aerobic bacteria in an aerobic environment; and
a sulfonamide in a pharmaceutically acceptable form selected from one or more of a salt, phosphate, acid, and ester, in which the sulfonamide is active against an aerobic bacteria in an aerobic environment, and may be substituted with an inhibitor bacterial dihydropteroate synthase that has some activity against an aerobic bacteria in an aerobic environment,
wherein the plurality of co-agents are provided in at least one period to a subject in need thereof causing one or more of the plurality of co-agents to act as a synergist and causing antibacterial activity against the one or more pathogenic anaerobes in the anaerobic environment, the at least one period being selected from a group consisting of about three hours or less, about two hours or less, and about 1 hour or less.

2. The plurality of co-agents of claim 1, wherein the plurality of co-agents are active against at least one of the one or more pathogenic anaerobes when the at least one of the one or more pathogenic anaerobes is in any of an acidic pH, a neutral pH, and a basic pH, wherein the acidic pH is a pH as low as about pH 5, and wherein the basic pH is a pH as high as about pH 8.

3. The plurality of co-agents of claim 1, wherein at least one of the one or more pathogenic anaerobes is considered resistant to at least one co-agent of the plurality of co-agents when that at least one co-agent is used singly against the at least one of the one or more pathogenic anaerobes, and wherein at least one of the one or more pathogenic anaerobes is an obligate anaerobic bacteria.

4. The plurality of co-agents of claim 1, wherein the plurality of co-agents are provided as pharmacotherapy to the subject, the plurality of co-agents being provided by co-dosing in the period, wherein the co-dosing is further selected from a group consisting of at a same time, at substantially a same time, in an overlapping expanse of time within the period, and in a series within the period, and wherein, in 24 hours, the co-dosing in the period is selected from one of the group consisting of once, twice, three times, four times, six times, eight times, twelve times, and continuously.

5. The plurality of co-agents of claim 1, wherein the plurality of co-agents are provided as pharmacotherapy to the subject having or suspected of having an infection caused by or suspected of being caused by one or more Gram-positive obligate anaerobic bacteria in an anaerobic environment.

6. The plurality of co-agents of claim 1, wherein the plurality of co-agents are provided as pharmacotherapy to the subject having or suspected of having an infection caused by or suspected of being caused by one or more Gram-negative obligate anaerobic bacteria in an anaerobic environment.

7. The plurality of co-agents of claim 1, wherein the plurality of co-agents are provided as pharmacotherapy to the subject having or suspected of having an infection caused by or suspected of being caused by one or more challenging anaerobes selected from a microaerophilic Streptococcus, including one or more of S. anginosus, S. constellatus, S. intermedius, S. mutans, and viridans streptococci, and the plurality of co-agents is active against the one or more microaerophilic Streptococcus.

8. The plurality of co-agents of claim 1, wherein the plurality of co-agents are provided as pharmacotherapy to the subject having or suspected of having an infection caused by or suspected of being caused by one or more Gram-positive obligate anaerobic bacteria selected from one or more in a group consisting of Actinomyces spp., Arcanobacterium spp., Atopobium spp., Bifidobacterium spp., Bilophila spp., Clostridioides spp., Clostridium spp., Collinsella spp., Eggerthella spp., Eubacterium spp., Finegoldia spp., Parvimonas spp., Peptococcus spp., Peptostreptococcus spp., Propionibacterium spp., and Cutibacterium spp., and the plurality of co-agents are active against the one or more one or more Gram-positive obligate anaerobic bacteria.

9. The plurality of co-agents of claim 1, wherein the plurality of co-agents are provided as pharmacotherapy to the subject in need thereof, the subject having or suspected of having an infection caused by or suspected of being caused by one or more Gram-negative obligate anaerobic bacteria selected from one or more in a group consisting of Aggretibacter spp., Bacteroides spp., Parabacteroides spp., Dethiosulfovibrio spp., Fusobacterium spp., Phocaeicola spp., Porphyromonas spp., Prevotella spp., Sutterella spp., Veillonella spp., and the plurality of co-agents is active against the one or more one or more Gram-negative obligate anaerobic bacteria.

10. The plurality of co-agents of claim 1, wherein the plurality of co-agents, when provided as pharmacotherapy to the subject, are not effective as an antibacterial against one or more commensal anaerobic bacteria species selected from a group consisting of Lactobacillus spp., Pediococcus spp., and Leuconostoc spp.

11. A synergist for an antibiotic combination, the antibiotic combination comprising:
a diaminopyridine in a pharmaceutically acceptable form selected from one or more of a salt, phosphate, acid, and ester, in which the diaminopyridine may be substituted for an inhibitor of bacterial dihydrofolate reductase, any of which will when used singly have: (a) some activity against an aerobic bacteria in an aerobic environment; and (b) no activity against one or more obligate anaerobic bacteria in an anaerobic environment; and
a sulfonamide in a pharmaceutically acceptable form selected from one or more of a salt, phosphate, acid, and ester, in which the sulfonamide may be substituted for an inhibitor of bacterial dihydropteroate synthase, any of which will when used singly have: (a) some activity against an aerobic bacteria in an aerobic environment; and (b) no activity against one or more obligate anaerobic bacteria in an anaerobic environment;
wherein the diaminopyridine and the inhibitor of bacterial dihydrofolate reductase will inhibit or inactive a dihydrofolate reductase enzyme of the one or more obligate anaerobic bacteria, and wherein the sulfonamide and the inhibitor of bacterial dihydropteroate synthase will inhibit or inactive a dihydropteroate synthase enzyme of the one or more obligate anaerobic bacteria, and
the synergist comprising at least:
a fosfomycin in a pharmaceutically acceptable form selected from one or more of a salt, phosphate, amine, acid, and ester, in which the fosfomycin may be substituted for an inhibitor of bacterial UDP-GlcNAc enolpyruvyl transferase (MurA), any of which will when used singly have: (a) some activity against an aerobic bacteria in an aerobic environment; and (b) no activity against one or more obligate anaerobic bacteria in an anaerobic environment;
wherein the synergist with the antibiotic combination causes synergistic action so as to transform therapeutic potential of the antibiotic combination, so that the synergist with the antibiotic combination are active against the one or more obligate anaerobic bacteria in the anaerobic environment.

12. The synergist of claim 11, wherein the synergist and the antibiotic combination are in a same formulation, and for synergistic action, the synergist and antibiotic combination are provided in a same period, the period being selected from one of a group consisting of within or less than about one hour, within or less than about two hours, and within or less then about three hours.

13. The synergist of claim 11, wherein the synergist and the antibiotic combination are in different formulations, and for synergistic action, the synergist and antibiotic combination are provided in a same period, the period being selected from one of a group consisting of within or less than about one hour, within or less than about two hours, and within or less then about three hours.

14. The synergist of claim 11, wherein the synergist and the antibiotic combination, as pharmacotherapy for a subject in need thereof, are provided to the subject in a period, the period for providing the synergist and the antibiotic combination being selected from one of a group consisting of a same period, a substantially same period, an overlapping expanse of time within the period, and in a series within the period.

15. The synergist of claim 11, wherein the fosfomycin or inhibitor of bacterial MurA is selected from one or more of a group consisting of a phosphonate, a phosphonic acid, a derivative of phosphonate, a derivative of phosphonic acid, a stereoisomer thereof, a geometric isomer thereof, a tautomer thereof, a hydrate thereof, a solvate thereof, and wherein a representative example of a fosfomycin is a hydrosoluble fosfomycin salt.

16. The synergist of claim 11, wherein the diaminopyridine or inhibitor of bacterial dihydrofolate reductase is selected from one or more of a group consisting of a 2,4-diaminopyrimidine, a derivative of 2,4-diaminopyrimidine, a stereoisomer thereof, a geometric isomer thereof, a tautomer thereof, a hydrate thereof, a solvate thereof, and wherein representative examples of a diaminopyridine are selected from one or more of trimethoprim, pyrimethamine, diaveridine, brodimoprim, tetroxoprim, metioprim, and iclaprim.

17. The synergist of claim 11, wherein the sulfonamide or inhibitor of bacterial dihydropteroate synthase is selected from one or more of a group consisting of a sulfanilamide, a derivative of sulfanilamide, a sulfam, a derivative of a sulfam, a sulfonamide, a derivative of a sulfonamide, a disulfonimide, a derivative of a disulfonimide, a stereoisomer thereof, a geometric isomer thereof, a tautomer thereof, a hydrate thereof, a solvate thereof, and wherein representative examples of a sulfonamide are selected from one or more of sulfadiazine, sulfamethoxazole, sulfatroxazole, sulfamerazine, sulfadoxine, sulfadimethoxine, sulfamethazine, sulfapyrazole, sulfaquinoxaline, sulfachloropyridazine, sulfaguanidine, sulfalene, sulfametin, sulfamethoxine, sulfamethoxypyridazine, sulfamethylphenazole, sulfamethoxypyridazine, sulfaethoxypyridazine, sulfabromomethazine, sulfaphenazole, sulfamoxole, sulfapyrazine, sulfapyridazine, sulfapyridine, sulfasymazine, sulfathiozole, sulfametrole, sulfanilimide, sulfasomidine, and sulfisoxazole.

18. The synergist of claim 11, wherein the synergist further comprises a co-agent, the co-agent being an inhibitor of a bacterial fosfomycin modifying (FME) enzyme selected from one or more of a group consisting of a phosphonoformate, a phosphonoacetate, a methylphosphonate, an ethylphosphonate, a phenylphosphonate, an acetylphosphonate, a phosphonoacetaldehyde, a stereoisomer thereof, a geometric isomer thereof, a tautomer thereof, a hydrate thereof, a solvate thereof, and wherein representative examples are selected from one or more of sodium phosphonoformate, sodium phosphonoformate tribasic hexahydrate, triethyl phosphonoformate, 2-phosphonobutyrate, 4-phosphonobutyrate, 2-phosphonoproprionate, 2-phosphonoproprionate, and 3-phosphonoproprionate, and wherein the inhibitor of the bacterial FME will inhibit or inactive FME of the one or more obligate anaerobic bacteria.

19. The synergist of claim 11, wherein the synergist further comprises a co-agent, the co-agent being one or more from a group of co-agents consisting of: (a) an inhibitor of bacterial peptidoglycan synthesis selected from a glycopeptide or lipoglycopeptide class of antibiotics, wherein inhibitor of bacterial peptidoglycan synthesis will bind to a lipid II precursor of an outer wall of the one or more obligate anaerobic bacteria, inhibiting peptidoglycan synthesis and inhibiting synthesis of a cell wall of the one or more obligate anaerobic bacteria; (b) a beta lactam antibiotic selected from a class of beta lactam antibiotics, wherein the beta lactam antibiotic will bind to a penicillin-binding protein enzyme and inhibit peptidoglycan synthesis of the one or more obligate anaerobic bacteria; and (c) an inhibitor of bacterial beta-lactamase, wherein the inhibitor of bacterial beta-lactamase will inhibit or inactive a beta-lactamase enzyme of the one or more obligate anaerobic bacteria, and wherein the co-agent is provided in a same period as the synergist and the antibiotic combination, the period being selected from one of a group consisting of within or less than about one hour, within or less than about two hours, and within or less then about three hours, and wherein the synergist and the antibiotic combination and the co-agent are, in 24 hours, provided for one of the group consisting of once, twice, three times, four times, six times, eight times, twelve times, and continuously.

20. A kit containing pharmaceutically acceptable co-agents for utilization as pharmacotherapy against one or more pathogenic anaerobes in an anaerobic environment, the kit comprising at least:
a first co-agent in a therapeutically effective amount for delivery of the first co-agent in 24 hours, the first co-agent being a fosfomycin in a pharmaceutically acceptable form selected from one or more of a salt, phosphate, acid, amine, and ester, wherein the first co-agent when used alone in an aerobic environment is an inhibitor of bacterial UDP-GlcNAc enolpyruvyl transferase;
a second co-agent in a therapeutically effective amount for delivery of the second co-agent in 24 hours, the second co-agent being a diaminopyridine in a pharmaceutically acceptable form selected from one or more of a salt, phosphate, acid, and ester, wherein the second co-agent when used alone in an aerobic environment is an inhibitor of bacterial dihydrofolate reductase;
a third co-agent in a therapeutically effective amount for delivery of the third co-agent in 24 hours, the third co-agent being a sulfonamide in a pharmaceutically acceptable form selected from one or more of a salt, phosphate, acid, and ester, wherein the second co-agent when used alone in an aerobic environment is an inhibitor bacterial dihydropteroate synthase; and
optionally at least one diluent for any one or more of the first co-agent, the second co-agent, and the third co-agent, wherein the first co-agent, the second co-agent and the third co-agent are in one or more vehicles, the one or more vehicles comprising a sufficient amount for at least one delivery in the 24 hours, wherein the kit is for delivery of a therapeutically effective amount of the first co-agent and a therapeutically effective amount of the second co-agent and therapeutically effective amount of the third co-agent for at least one delivery to a subject for treating or preventing an infection from one or more obligate anaerobic bacteria.
